SEQUENCE LISTING (9 sequences)

SEQ ID NO: 1 - protein, 5 aa, Artificial Sequence
  Synthetic peptide
SRGDS

SEQ ID NO: 2 - protein, 4 aa, Artificial Sequence
  Synthetic peptide
RGDS

SEQ ID NO: 3 - protein, 13 aa, Artificial Sequence
  Synthetic peptide
AGQWHRVSVRWGC

SEQ ID NO: 4 - protein, 7 aa, Artificial Sequence
  Synthetic peptide
SVVYGLR

SEQ ID NO: 5 - protein, 4 aa, Artificial Sequence
  Synthetic peptide
DGEA

SEQ ID NO: 6 - protein, 8 aa, Artificial Sequence
  Synthetic peptide
IRIKIRIK

SEQ ID NO: 7 - protein, 12 aa, Artificial Sequence
  Synthetic peptide
IKKIIKKIIKKI

SEQ ID NO: 8 - protein, 9 aa, Artificial Sequence
  Synthetic peptide
  MOD_RES (3)..(3): Hyp
  MOD_RES (6)..(6): Hyp
  MOD_RES (9)..(9): Hyp
GPXGPXGPX

SEQ ID NO: 9 - protein, 9 aa, Artificial Sequence
  Synthetic peptide
  MOD_RES (2)..(2): Hyp
  MOD_RES (5)..(5): Hyp
  MOD_RES (8)..(8): Hyp
PXGPXGPXG

(19) United States
(12) Patent Application Publication
(10) Pub. No.: US 2023/0399457 A1
(43) Pub. Date: Dec. 14, 2023

(54) Bioactive Synthetic Copolymer, Bioactive Macromolecule and Related Methods Thereof

(71) Applicant: Agency for Science, Technology and Research, Singapore (SG)

(72) Inventors: Peili Teo, Singapore (SG); Jiayi Guo, Singapore (SG); Yew Chin Teo, Singapore (SG); Eun Ju Park, Singapore (SG)

(21) Appl. No.: 18/251,144

(22) PCT Filed: Oct. 30, 2020
(86) PCT No.: PCT/SG2020/050621

§ 371 Date: Apr. 28, 2023

(51) Int. Cl. C08G 61/08

(52) U.S. Cl. CPC C08G 61/08; C08G 2261/122; C08G 2261/1426; C08G 2261/1432; C08G 2261/3342; C08G 2261/418

(57) ABSTRACT

There is provided a bioactive synthetic copolymer with a poly(norbornene) backbone comprising one or more repeating units represented by general formula (I) and one or more repeating units represented by general formula (II). Also provided are a bioactive macromolecule, a material comprising said bioactive synthetic copolymer, a method of preparing said bioactive synthetic copolymer and a method of preparing said bioactive macromolecule.

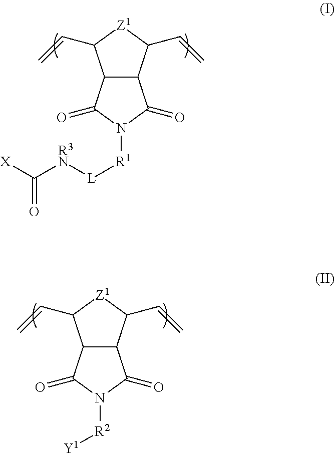

### TECHNICAL FIELD

The present disclosure relates broadly to a bioactive synthetic copolymer, a bioactive macromolecule and a material comprising said bioactive synthetic copolymer. The present disclosure also relates to methods of preparing said bioactive synthetic copolymer, said bioactive macromolecule and said material.

### BACKGROUND

A better understanding of the biology and physiology of living things over the years has led to an appreciation of the potential of using alternative materials to enhance or replace existing functions in biological systems.

However, identifying a suitable material that meets both the mechanical and biological requirements to function desirably in or with biological systems is often challenging.

This is because bioactive molecules (e.g. collagen, chitosan etc) that have the desired biological attributes often lack the mechanical strength needed for a useful biomedical application. For example, many of such bioactive molecules are very hygroscopic and exist as gels upon absorption of moisture, rendering them too weak for use in weight bearing biomedical applications such as implantable devices on their own.

On the other hand, synthetic materials that have mechanically superior characteristics lack the biological attributes needed for them to be properly used in applications that require constant interaction with biological systems. For example, a number of such synthetic material can induce foreign body reaction (FBR) when inserted into a human body. This can result in inflammation and other types of undesired immune responses being elicited around the implantation site.

Combining these different materials with the hope that the resultant material obtained can achieve both the desired biological and mechanical properties is also challenging. This is because bioactive molecules such as peptides and carbohydrates are often incompatible with synthetic polymers since the former is hydrophilic whereas the latter is hydrophobic.

Thus, physically blending the two different materials together often result in phase separation of the two mutually incompatible materials, rendering the obtained entire material ineffective.

The inherent differences in their hydrophilicity likewise make chemically synthesising a bioactive polymer from these materials extremely difficult, especially when the molecular weights of these materials are relatively high. This is in addition to the various complex chemical hurdles (e.g. potentially high intramolecular reactivity, unwanted chemical leaching of by-products etc) that need to be overcome when attempting to chemically combine these two chemically different types of materials together.

In view of the above, there is a need to address or at least ameliorate the above-mentioned problems. In particular, there is a need to provide a bioactive synthetic copolymer, a bioactive macromolecule, a material comprising said bioactive synthetic copolymer and related methods that address or at least ameliorate the above-mentioned problems.

### SUMMARY

In one aspect, there is provided a bioactive synthetic copolymer with a poly(norbornene) backbone comprising one or more repeating units represented by general formula (I) and one or more repeating units represented by general formula (II):

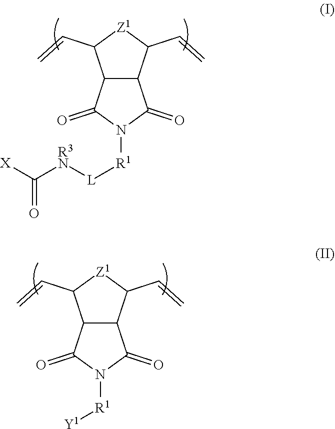

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y¹ comprises a synthetic polymer or parts thereof; and

Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

In one embodiment, the molecular weight of general formula (I) do not differ from the molecular weight of general formula (II) by more than 30% of the molecular weight of general formula (11).

In one embodiment, L is heteroalkylene having from 20 carbon atoms to 300 carbon atoms.

In one embodiment, L is polyethylene glycol (PEG).

In one embodiment, L is polyethylene glycol (PEG) having a number average molecular weight of between 500 and 7,000.

In one embodiment, R¹ is C₁-C₄ alkyl and R² is selected from C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl or C₃-C₂₀ alkylcarbonylalkyl.

In one embodiment, R¹ is straight or branched C₁-C₄ alkyl substituents independently selected from methyl, ethyl, n-propyl, 2-propyl, isopropyl, n-butyl, isobutyl, sec-butyl or t-butyl, and R² is straight or branched C₁-C₂₀ alkyl substituents independently selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl or decyl.

In one embodiment, Z¹ and Z² are both CR^{a}R^{b} wherein R^{a} and R^{b} are each independently selected from the group consisting of H, optionally substituted alkyl, optionally substituted alkenyl and optionally substituted alkynyl.

In one embodiment, X comprises protein, peptide or carbohydrate selected from the group consisting of peptide sequence, laminin-derived peptide, integrin binding peptide, cell-penetrating peptide, collagen mimics, collagen fragments, heparin sulfate, glycosaminoglycans (GAGs) and derivatives thereof.

In one embodiment, X is selected from the group consisting of RGD, SRGDS, RGDS, A5G81 (AGQWHRVSVRWGC), SVVYGLR, (IRIK)₂, (IKKI)₃, heparin oligosaccharide DP8, DP10, DP12, DP14, DP16, DGEA, (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence and hyaluronic acid.

In one embodiment, X comprises an antibiotic, antimicrobial, antibacterial moiety, blood thinning agents or anti-inflammatory agents.

In one embodiment, X comprises antibiotic, antimicrobial, antibacterial, blood thinning agents or anti-inflammatory agents selected from the group consisting of penicillin, amoxicillin, amphotericin, ciprofloxacin (CIF), atorvastatin, aspirin, streptomycin, ribostamycin and gentamycin.

In one embodiment, Y¹ is represented by general formula (III):

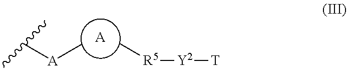

wherein

A is selected from a single bond, oxy, carbonyl, oxycarbonyl, carboxyl, optionally substituted alkoxy, optionally substituted alkoxyalkyl, optionally substituted alkylcarbonyl, optionally substituted alkylcarbonylalkyl, optionally substituted carboxyalkyl, optionally substituted oxycarbonylalkyl, optionally substituted alkylcarboxylalkyl, or optionally substituted alkoxycarbonylalkyl;

B is optionally present as a ring selected from 1,2,3-triazole or succinimide:

R⁵ is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

Y² is selected from the group consisting of polypropylene (PP), polyesters, poly(lactic acid) (PLA), poly(lactic-co-glycolic acid) (PLGA), poly(caprolactone) (PCL), polystyrene (PS), polyacrylates, poly(meth)acrylates, polyamides (PA), and parts thereof; and

T is a terminal group selected from the group consisting of hydrogen, halogen, hydroxyl, amino, acyl, thiol, optionally substituted alkyl, optionally substituted alkenyl, optionally substituted alkynyl, optionally substituted alkoxy, optionally substituted alkoxyalkyl, optionally substituted alkylcarbonyl, optionally substituted alkylcarbonylalkyl, optionally substituted carboxyalkyl, optionally substituted oxycarbonylalkyl, optionally substituted alkylcarboxylalkyl or optionally substituted alkoxycarbonylalkyl.

In one embodiment, Y¹ is selected from the following general formulae (IIIa), (IIIb), (IIIc), (IIId), (IIIe) or (IIIf):

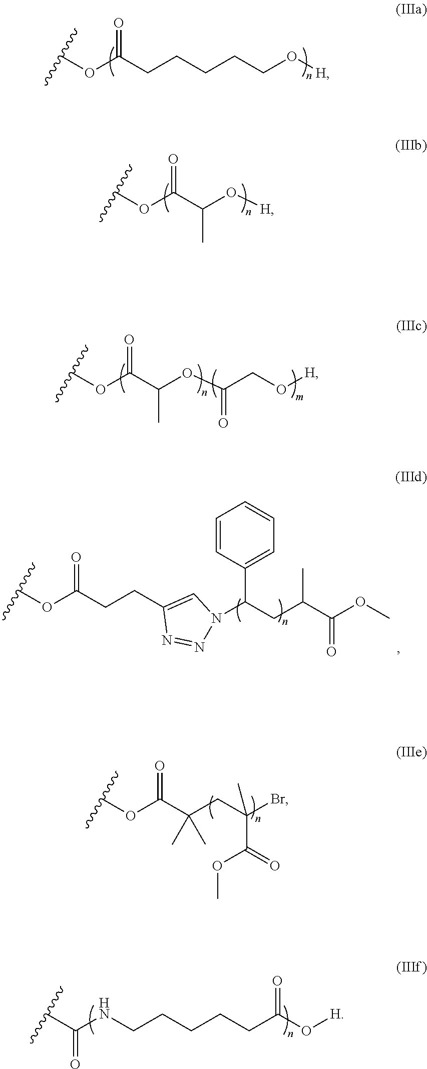

In one aspect, there is provided a method of preparing a bioactive synthetic copolymer disclosed herein, the method comprising:

polymerising one or more bioactive macromolecules represented by general formula (IV) with one or more synthetic macromolecules represented by general formula (V) in the presence of a catalyst to obtain the bioactive synthetic copolymer:

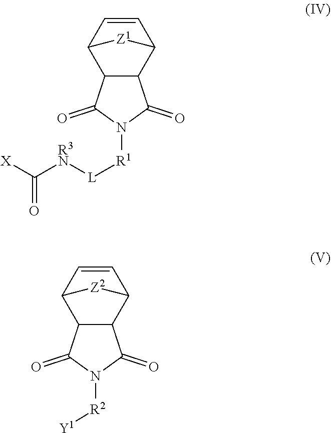

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y¹ comprises a synthetic polymer or parts thereof; and

Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

In one embodiment, the catalyst comprises a ruthenium complex.

In one embodiment, the method comprises ring opening metathesis polymerisation (ROMP).

In one aspect, there is provided a bioactive macromolecule represented by general formula (IV) for preparing the copolymer disclosed herein:

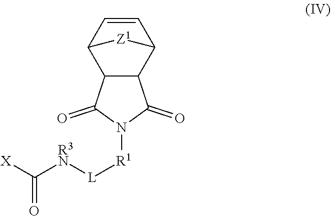

wherein

R¹ is optionally substituted alkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof; and

Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

In one aspect, there is provided a method of preparing a bioactive macromolecule disclosed herein, the method comprising:
(i) providing a dicarboxylic anhydride having general formula (VI):

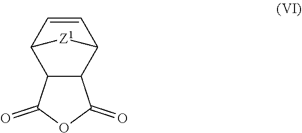

wherein
Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl;
(ii) reacting said dicarboxylic anhydride having general formula (VI) with a diamine R⁴R³N-L-R¹—NH₂ to obtain an amine having general formula (VII):

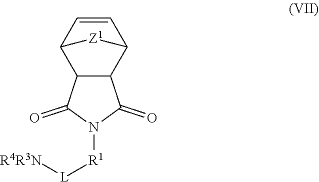

wherein
R¹ is optionally substituted alkyl;
R³ and R⁴ are each independently selected from H, optionally substituted alkyl, optionally substituted alkenyl or optionally substituted alkynyl, wherein at least one of R³ and R⁴ is H;

L is heteroalkylene; and

Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl;
(iii) reacting said amine having general formula (VII) with an acid-containing bioactive moiety X—C(═O)OH to obtain the bioactive macromolecule, wherein X comprises a bioactive moiety selected from the group consisting of proteins, peptides, carbohydrates, therapeutic/drug molecules and derivatives thereof.

In one embodiment, the method further comprises, prior to the step of reacting amine having general formula (VII) with X—C(═O)OH, purifying the amine having general formula (VII) to remove impurities.

In one embodiment, the step of purifying comprises double neutralisation steps.

In one embodiment, the double neutralisation steps comprise a first step of washing with acid and a second step of washing with base.

In one aspect, there is provided a material comprising a copolymer disclosed herein for use in medicine.

In one embodiment, the material is part of an apparatus selected from the group consisting of wound dressing, skin scaffold, bone scaffold, organoid scaffold, implants, and medical device.

### Definitions

The term “polymer” as used herein refers to a chemical compound comprising repeating units and is created through a process of polymerization. The units composing the polymer are typically derived from monomers and/or macromonomers. A polymer typically comprises repetition of a number of constitutional units.

The terms “monomer” or “macromonomer” as used herein refer to a chemical entity that may be covalently linked to one or more of such entities to form a polymer.

The term “bioactive” as used herein broadly refers to the property of having a biological effect, preferably a desirable or positive biological effect on a living organism, tissue, or cell.

The term “biocompatible” as used herein broadly refers to a property of being compatible with biological systems or parts of the biological systems without substantially or significantly eliciting an adverse physiological response such as a toxic reaction, an immune reaction, an injury or the like. Such biological systems or parts include blood, cells, tissues, organs or the like.

The term “bond” refers to a linkage between atoms in a compound or molecule. The bond may be a single bond, a double bond, or a triple bond.

In the definitions of a number of substituents below, it is stated that “the group may be a terminal group or a bridging group”. This is intended to signify that the use of the term is intended to encompass the situation where the group is a terminal group/moiety as well as the situation where the group is a linker between two other portions of the molecule. Using the term “alkyl” having 1 carbon atom as an example, it will be appreciated that when existing as a terminal group, the term “alkyl” having 1 carbon atom may mean —CH₃ and when existing as a bridging group, the term “alkyl” having 1 carbon atom may mean —CH₂— or the like.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “heteroalkylene” as used herein refers to alkylene having one or more —CH₂— replaced with a heteroatom selected from O, NR, Si, P or S, where R is hydrogen or alkyl as defined herein. The term “heteroalkylene” can be linear, branched or cyclic and containing up to 500 carbon atoms.

The term “alkoxy” as used herein refers to straight chain or branched alkyloxy groups. Examples include methoxy, ethoxy, n-propoxy, isopropoxy, tert-butoxy, and the like.

The term “alkoxyalkyl” as used herein is intended to broadly refer to a group containing —R—O—R′, where R and R′ are alkyl as defined herein. The group may be a terminal group or a bridging group.

The term “alkylcarbonyl” as used herein is intended to broadly refer to a group containing —R—C(═O)—, where R is alkyl as defined herein. The group may be a terminal group or a bridging group.

The term “alkylcarbonylalkyl” as used herein is intended to broadly refer to a group containing —R—C(═O)—R′, where R and R′ are alkyl as defined herein. The group may be a terminal group or a bridging group.

The term “carboxylalkyl” as used herein is intended to broadly refer to a group containing —C(═O)—O—R, where R is alkyl as defined herein. The group may be a terminal group or a bridging group.

The term “oxycarbonylalkyl” as used herein is intended to broadly refer to a group containing —O—C(═O)—R, where R is alkyl as defined herein. The group may be a terminal group or a bridging group.

The term “alkylcarboxylalkyl” as used herein is intended to broadly refer to a group containing —R—C(═O)—O—R′, where R and R′ are alkyl as defined herein.

The group may be a terminal group or a bridging group.

The term “alkoxycarbonylalkyl” as used herein is intended to broadly refer to a group containing —R—O—C(═O)—R′, where R and R′ are alkyl as defined herein. The group may be a terminal group or a bridging group.

The term “oxy” as used herein is intended to broadly refer to a group containing —O—.

The term “carbonyl” as used herein is intended to broadly refer to a group containing —C(═O)—.

The term “oxycarbonyl” as used herein is intended to broadly refer to a group containing —O—C(═O)—.

The term “carboxyl” as used herein is intended to broadly refer to a group containing —C(═O)—O—R, where R is hydrogen or an organic group.

The term “halogen” represents chlorine, fluorine, bromine or iodine. The term “halo” represents chloro, fluoro, bromo or iodo.

The term “amine group” or the like is intended to broadly refer to a group containing —NR₂, where R is independently a hydrogen or an organic group. The group may be a terminal group or a bridging group.

The term “amide group” or the like is intended to broadly refer to a group containing —C(═O)NR₂, where R is independently a hydrogen or an organic group. The group may be a terminal group or a bridging group.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The term “micro” as used herein is to be interpreted broadly to include dimensions from about 1 micron to about 1000 microns.

The term “nano” as used herein is to be interpreted broadly to include dimensions less than about 1000 nm, less than about 500 nm, less than about 100 nm or less than about 50 nm.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship.

For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a bioactive synthetic copolymer, a bioactive macromolecule for preparing the bioactive synthetic copolymer, a material comprising the bioactive synthetic copolymer and related methods are disclosed hereinafter.

### Bioactive Synthetic Copolymer

There is provided a bioactive synthetic copolymer with a poly(norbornene) backbone comprising one or more repeating units represented by general formula (I) and one or more repeating units represented by general formula (II):

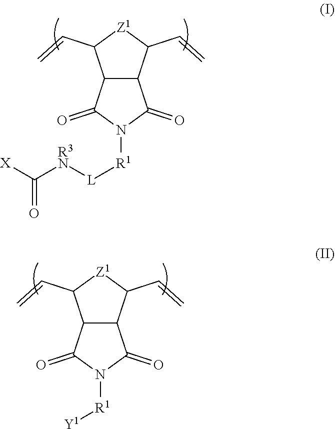

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y¹ comprises a synthetic polymer or parts thereof; and

Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

In various embodiments, the repeating unit(s) represented by general formula (I) and/or moiety X possess bioactivity, biocompatibility and/or biodegradability. In various embodiments, the repeating unit(s) represented by general formula (II) and/or moiety Y¹ possess good mechanical strength/hardness. In various embodiments, the repeating unit represented by general formula (II) and/or moiety Y¹ has a higher mechanical strength than the repeating unit represented by general formula (I) and/or moiety X. Advantageously, the presence of repeating units represented by general formulae (I) and (II) in the bioactive synthetic copolymer imparts both bioactivity and mechanical strength to the copolymer, leading to a mechanically strong bioactive copolymer. In various embodiments, the copolymer may also be biocompatible and/or biodegradable. Accordingly, in various embodiments, the copolymer is capable of being classified as a biomaterial. Advantageously, due to the presence of synthetic and bioactive side chains, the bioactive synthetic copolymer may also have a higher thermal stability than conventional biomolecules such as peptides, proteins, carbohydrates or glycosaminoglycans. Even more advantageously, the thermal stability of the bioactive synthetic copolymer allows for embodiments of the copolymer to be suitable for processing at high temperatures or even harsh material processing such as melt extrusion >200° C., making the copolymer ideal/attractive for use in applications such as biomedical devices. In various embodiments, the synthetic polymer is substantially or completely non-bioactive, or at least less bioactive than the bioactive moiety.

In various embodiments, L is a polymeric linker that links the bioactive moiety X to the poly(norbornene) backbone. Advantageously, L is designed to be adjustable and/or customizable based on the size of the bioactive moiety X and the size of the synthetic polymer present in Y¹. The molecular weight and/or length of the polymeric linker L may be customized to suit the molecular weight and/or length of the bioactive moiety X and synthetic polymer chosen for Y¹, depending on the application the copolymer is to be used for. For example, in skin scaffolds, shorter synthetic polymeric (e.g., PCL or PLA) side chains are preferred for fast degradation whereas in bone scaffolds, longer synthetic polymeric (e.g., PCL or PLA) side chains are selected for slower degradation in body. Without being bound by theory, it is believed that bone tissues are expected to grow slower than skin tissues, hence the bone scaffold needs to stay intact in the body for a longer period of time for bone tissues to regenerate and cannot degrade too quickly. For example, for applications in dressings, or particularly non-biodegradable non-woven fibers which require thermal stability and/or mechanical strength properties, low molecular weight is preferred for synthetic polymers due to their poor solubility in common solvents. In various embodiments, synthetic polymers having low molecular weight comprises synthetic polymers having molecular weight of no more than about 5,000, for example when the synthetic polymers are highly insoluble, e.g. polyamide (PA). In other embodiments, synthetic polymers having a molecular weight of no more than about 10,000 may be used/acceptable, for example, when the synthetic polymers are less insoluble.

In various embodiments, the molecular weight and/or length of the polymeric linker L is selected such that the overall molecular size of the repeating unit represented by general formula (I) is similar/comparable to the molecular size of the repeating unit represented by general formula (II). For example, if PCL having a molecular weight of 4,000 is selected as the choice of synthetic polymer for Y¹ and peptide having a molecular weight of from about 400 to about 500 is selected as the choice of bioactive moiety X, then L may be designed to comprise a molecular weight of about 3,400. It will be appreciated that in various embodiments, it is the length of L that gets adjusted to match the molecular weight of general formula (I) to molecular weight of general formula (II).

In various embodiments, the molecular weight of general formula (I) is comparable/substantially similar with/to the molecular weight of general formula (II). In various embodiments, the molecular weight of general formula (I) does not differ from the molecular weight of general formula (II) by more than 30% of the molecular weight of general formula (II) or vice versa. For example, the molecular weight of general formula (I) may be at most about 30% more or at most 30% less than the molecular weight of general formula (II) or vice versa. The molecular weight of general formula (I) may not differ from the molecular weight of general formula (II) by more than about 30%, more than about 25%, more than about 20%, more than about 15%, more about 10%, more than about 5%, more than about 4%, more than about 3%, more than about 2%, or more than about 1% of the molecular weight of general formula (II) or vice versa. In various embodiments, the molecular weight of general formula (I) does not differ from the molecular weight of general formula (II) by more than about 20% of the molecular weight of general formula (II) or vice versa. For example, the molecular weight of general formula (I) may be at most about 20% more or at most 20% less than the molecular weight of general formula (II) or vice versa. Advantageously, as the bioactive moiety bearing repeating unit has a molecular size/weight/length that is similar to that of the synthetic polymer bearing repeating unit, the length of the bioactive moiety X is extended, thereby allowing X to be “visible”, available for binding to cells or accessible to its targeted physiological site for desired bioactivity, i.e. not buried in a sea/matrix of synthetic polymers.

In various embodiments, the molecular weight of general formula (I) is about 15,000, about 14,000, about 13,000 or at least about 12,000. In various embodiments, the molecular weight of general formula (I) is from about 100 to about 15,000, from about 200 to about 14,000, from about 300 to about 13,000, from about 400 to about 12,000, from about 500 to about 11,000, from about 1,000 to about 10,000, from about 1,500 to about 9,500, from about 2,000 to about 9,000, from about 2,500 to about 8,500, from about 3,000 to about 8,000, from about 3,500 to about 7,500, from about 4,000 to about 7,000, from about 4,500 to about 6,500, from about 5,000 to about 6,000 or about 5,500. In various embodiments, when X comprises longer peptides that contain more than 10 amino acids and the molecular weight of L is about 6,000, then the molecular weight of general formula (I) is greater than about 7,000.

In various embodiments, the molecular weight of general formula (II) is from about 100 to about 15,000, from about 200 to about 14,000, from about 300 to about 13,000, from about 400 to about 12,000, from about 500 to about 11,000, from about 1,000 to about 10,000, from about 1,500 to about 9,500, from about 2,000 to about 9,000, from about 2,500 to about 8,500, from about 3,000 to about 8,000, from about 3,500 to about 7,500, from about 4,000 to about 7,000, from about 4,500 to about 6,500, from about 5,000 to about 6,000 or about 5,500.

In various embodiments, the total molecular weight of general formula (I) and general formula (II) is kept to about 300,000, no more than about 300,000, no more than about 200,000, no more than about 100,000, no more than about 90,000, no more than about 80,000, no more than about 70,000, no more than about 60,000, no more than about 50,000, no more than about 45,000, no more than about 40,000, no more than about 35,000, no more than about 30,000, no more than about 25,000, no more than about 20,000, or no more than about 15,000 to facilitate copolymerisation.

In various embodiments, L is hydrophilic. As L is adjustable, the hydrophilicity of the repeating unit represented by general formula (I) and also the overall hydrophilicity of the bioactive synthetic copolymer may be adjusted as desired. Advantageously, the presence of L increases the hydrophilicity of the repeating unit represented by general formula (I) and also the overall hydrophilicity of the bioactive synthetic copolymer. Even more advantageously, the presence of L increases the hydrophilicity of the bioactive synthetic copolymer, therefore softening the synthetic polymeric chains which are hydrophobic, making the copolymer less stiff after processing. It will be appreciated by a person skilled in the art that, bioactive moieties and synthetic polymers are typically mutually incompatible as the individual bioactive moiety is generally hydrophilic while synthetic polymer is generally hydrophobic. Advantageously, L in repeating unit represented by general formula (I) is also used to extend the chain length of the bioactive moiety X attached at the end of L.

In various embodiments, L is amorphous. Advantageously, the presence of L increases the amorphousness and/or decreases the crystallinity of the bioactive synthetic copolymer, making the copolymer useful for crafting softer or less stiff plastics such as polystyrene-based material.

In various embodiments, L is a heteroalkylene having at least 20 carbon atoms, at least 30 carbon atoms, at least 40 carbon atoms, at least 50 carbon atoms, at least 60 carbon atoms, at least 70 carbon atoms, at least 80 carbon atoms, at least 90 carbon atoms, at least 100 carbon atoms, at least 150 carbon atoms, at least 200 carbon atoms, at least 250 carbon atoms or at least 300 carbon atoms. In various embodiments, L is C₂₀-C₃₀₀ heteroalkylene or a heteroalkylene having from 20 carbon atoms to 300 carbon atoms.

In various embodiments, L has a number average molecular weight of between about 500 and about 7,000. L may have a number average molecular weight of about 600, about 700, about 800, about 900, about 1,000, about 1,500, about 2,000, about 2,500, about 3,000, about 3,500, about 4,000, about 4,500, about 5,000, about 5,500, about 6,000, about 6,500 or about 7,000. In various embodiments, when X comprises a small bioactive moiety, the molecular weight of L may be adjusted to about 7,000 so that the total molecular weight of general formula (I) and general formula (II) is kept to no more than about 10,000. In various embodiments, the number average molecular weight of L is from about 1,000 to about 6,000.

In various embodiments, the heteroatom in L is O. In various embodiments, L is polyalkylene glycol. In various embodiments, L is poly(C₂-C₄ alkylene glycol). L may be selected from the group consisting of polyethylene glycol (PEG), polypropylene glycol (PPG), polytetramethylene glycol (PTMG), polybutylene glycol (PBG) and the like. Advantageously, the use of a polyalkylene glycol such as PEG can increase hydrophilicity of the macromonomer and the resultant copolymer. In various embodiments, the polyalkylene glycol such as PEG are used as spacers, linkers or linking groups in the overall polymers, instead of as terminal groups.

In various embodiments, L is polyalkylene glycol having at least about 10 repeating units, at least about 15 repeating units, at least about 20 repeating units, at least about 21 repeating units, at least about 22 repeating units, at least about 23 repeating units, at least about 24 repeating units, at least about 25 repeating units, at least about 30 repeating units, at least about 40 repeating units, at least about 50 repeating units, at least about 60 repeating units, at least about 70 repeating units, at least about 80 repeating units, at least about 90 repeating units, at least about 100 repeating units, at least about 150 repeating units, at least about 200 repeating units, or at least about 250 repeating units. In various embodiments, L comprises from about 10 monomers/repeating units to about 250 monomers/repeating units. Unlike conventional polymers which uses a short PEG chain, embodiments of the bioactive synthetic copolymer disclosed herein incorporate a long polyalkylene glycol chain of at least 21 repeating units at L.

In various embodiments, L is selected from the group consisting of PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₂₀₀₀, PEG₂₅₀₀, PEG₃₀₀₀, PEG₃₅₀₀, PEG₄₀₀₀, PEG₄₅₀₀, PEG₅₀₀₀, PEG₅₅₀₀, PEG₆₀₀₀, PEG₆₆₀₀ and mixtures thereof.

In various embodiments, X is coupled to the poly(norbornene dicarboximide) backbone through a carboxylic acid functionality in the following arrangement: —R¹-L-NR³—C(═O)—X. Advantageously, by linking X through a carboxylic acid functionality, amine terminal group(s) in X is/are free up for delivering its bioactivity, therefore ensuring the bioavailability of X. It will be appreciated that as amine group(s) confer bioactivity, exhausting up amine groups in bioactive moieties for polymer binding may be undesirable.

In various embodiments, X is coupled to the poly(norbornene dicarboximide) backbone via peptide/amide linkage, i.e. —NR³—C(═O)—. Advantageously, the bioactive synthetic copolymer disclosed herein is considerably stronger and/or stable than conventional polymers that contain ester linkages. Without being bound by theory, it is believed that amide linkages are stronger than ester linkages because ester linkages are more prone to hydrolysis, which may release bioactive moieties into the bloodstream, leading to a premature metabolism of bioactive moieties.

In various embodiments, one or more of H atoms in alkyl, alkenyl, alkynyl, alkoxyalkyl, alkylcarbonyl and alkylcarbonylalkyl is/are optionally replaced by hydroxy, hydroxyalkyl, halogen, haloalkyl, cyano, cyanoalkyl and nitro.

In various embodiments, R¹ is selected from C₁-C₂₀ alkyl. The C₁-C₂₀ alkyl substituents may be straight or branched substituents selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl or the like. R¹ may be straight or branched C₁-C₄ alkyl substituents. In various embodiments, the length of R¹ is the same as the length of a repeating unit in L. For example, if L is poly(butylene glycol), then R¹ is butyl. In another example, if L is poly(ethylene glycol), then R¹ is ethyl. It will be appreciated that in various embodiments, R¹ is carefully designed to match L.

In various embodiments, R³ is selected from H, C₁-C₂₀ alkyl, C₂-C₂₀ alkenyl or C₂-C₂₀ alkynyl.

In various embodiments, Z¹ and Z² are each independently selected from CH₂, O, NH, SiR^{a}R^{b}, PR^{a} or S. The poly(norbornene) backbone may be selected from the group consisting of poly(norbornene-imide), poly(norbornene-dicarboximide), poly(norbornene) backbone is poly(5-norbornene-2,3-dicarboximide), poly(7-oxanorbornene), poly(oxanorbornene-imide), poly(oxanorbornene-dicarboximide) and the like. In various embodiments, Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, C₁-C₂₀ alkyl, C₁-C₂₀ alkenyl and C₁-C₂₀ alkynyl. In various embodiments, Z¹ is CH₂. In various embodiments, Z² is CH₂.

In various embodiments, X comprises a bioactive moiety selected from proteins, peptides, carbohydrates, therapeutic/drug molecules and derivatives thereof. In various embodiments, proteins, peptides, carbohydrates or therapeutic/drug molecules derivatives thereof include proteins, peptides, carbohydrates or therapeutic/drug molecules that are or have been optionally modified to contain one carboxylic acid terminal group. In some embodiments, the bioactive moiety contains only one carboxylic acid terminal group.

In various embodiments, the bioactive moiety comprises a monocarboxylic acid. Advantageously, the use of a bioactive moiety having a monocarboxylic acid terminal group avoids the possibility of an undesirable crosslinking which may otherwise occur if there is more than one carboxylic acid. In various embodiments therefore, the bioactive moiety X is substantially devoid of more than one carboxylic acid terminal group, for e.g., a dicarboxylic acid or tricarboxylic acid.

In various embodiments, X comprises protein or peptide. X may be a peptide sequence, laminin-derived peptide, integrin binding peptide, cell-penetrating peptide, collagen mimics or collagen fragments. In various embodiments, X comprises from 2 to 50 amino acid residues, from 2 to 40 amino acid residues or from 2 to 20 amino acid residues in any sequence. In various embodiments, X comprises 50 amino acid residues, 40 amino acid residues, 30 amino acid residues, 25 amino acid residues, 20 amino acid residues, 15 amino acid residues, 10 amino acid residues, 9 amino acid residues, 8 amino acid residues, 7 amino acid residues, 6 amino acid residues, 5 amino acid residues, 4 amino acid residues or 3 amino acid residues in any sequence. The amino acid residues may be selected from the group consisting of glycine, alanine, valine, leucine, isoleucine, methionine, proline, phenylalanine, tryptophan, asparagine, glutamine, glycine, serine, threonine, serine, asparagine, glutamine, tyrosine, cysteine, lysine, arginine, histidine, aspartic acid and glutamic acid. In various embodiments, X is a peptide sequence comprising 3 to 20 natural amino acids. X may be integrin binding peptide selected from the group consisting of arginine-glycine-aspartic acid (RGD), SRGDS and RGDS; laminin-derived peptide A5G81 (AGQWHRVSVRWGC); osteopontin derived peptides SVVYGLR; and cell-penetrating/antimicrobial peptide selected from (IRIK)₂ or (IKKI)₃. In various embodiments, X is a collagen sequence comprising 3 to 20 units of glycine (G), proline (P) and hydroxyproline (Hyp) in any sequence or permutation. X may be collagen fragment having a (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence or collagen mimic DGEA.

In various embodiments, X comprises carbohydrate. In various embodiments, X comprises monosaccharide, disaccharide, oligosaccharide or polysaccharide. In various embodiments, X comprises from 2 to 50 saccharide units, from 2 to 40 saccharide units, from 2 to 20 saccharide units or from 10 to 14 saccharide units. In various embodiments, X comprises 50 saccharide units, 40 saccharide units, 30 saccharide units, 25 saccharide units, 20 saccharide units, 15 saccharide units, 14 saccharide units, 13 saccharide units, 12 saccharide units, 11 saccharide units, 10 saccharide units, 9 saccharide units, 8 saccharide units, 7 saccharide units, 6 saccharide units, 5 saccharide units, 4 saccharide units or 3 saccharide units or 2 saccharide units. X may be heparin sulfate (HS) or glycosaminoglycans (GAGs). In various embodiments, X is heparin sulfate/oligosaccharide selected from the group consisting of DP8, DP10, DP12, DP14 and DP16. In various embodiments, X is hyaluronic acid which is the simplest form of glycosaminoglycan (GAG).

In various embodiments, X is chemically coupled to the rest of general formula (I) via its hydroxy group. For example, when X is carbohydrate/saccharide, oxidation and/or reductive amination reactions may be performed on the carbohydrate's hydroxy for linking X to general formula (I). —CH₂OH on the saccharide may be oxidised to —C(═O)H, which subsequently undergoes reductive amination using the —NH₂ terminal on L to create a peptide linkage.

In various embodiments, X comprises a carbohydrate/saccharide that contained or has been modified to contain one carboxylic acid terminal group. Modification by one or more chemical reaction(s) such as oxidation may be performed on the carbohydrate/saccharide to create a carboxylic acid group. In various embodiments, modification is performed on a hydroxyl group that is originally present in the carbohydrate/saccharide. In various embodiments, —CH₂OH on the carbohydrate/saccharide is oxidized completely to —C(═O)OH, which subsequently reacts with a —NH₂ terminal on L to create a peptide linkage that links the carbohydrate/saccharide to the rest of general formula (I): X—C(═O)—NH-L-. It will be appreciated, however, that no modification to the carbohydrate/saccharide may be required/necessary if a carboxylic acid is naturally present in the carbohydrate/saccharide.

In various embodiments, X comprises therapeutic/drug molecule. In various embodiments, X comprises antibiotic, antimicrobial, antibacterial, blood thinning agents or anti-inflammatory agents. X may be penicillin, amoxicillin, amphotericin, ciprofloxacin (CIF), atorvastatin, aspirin or aminoglycoside-based molecules selected from streptomycin, ribostamycin or gentamycin. It will be appreciated that X may be any therapeutic or drug molecule that contains a carboxylic acid group.

In various embodiments, X is chemically coupled to the rest of general formula (I) via one of its chemical moiety selected from the group consisting of —COOH, —CH₂OH, —CH₂NH₂ and ═CHNH₂. For example, —CH₂NH₂ or ═CHNH₂ on the drug molecule may be coupled to a small dicarboxylic acid before reacting with a —NH₂ terminal on L to create a peptide linkage that links the drug molecule to the rest of general formula (I): X—C(═O)—NH-L-.

In various embodiments, X comprises a therapeutic/drug molecule that contained or has been modified to contain one carboxylic acid terminal group. Modification by one or more chemical reaction(s) such as oxidation may be performed on the therapeutic/drug molecule to create a carboxylic acid group. In various embodiments, modification is performed on a hydroxyl group that is originally present in the therapeutic/drug molecule. For example, in various embodiments when X is ribostamycin or gentamycin, —CH₂OH on the drug molecule is oxidized completely to —C(═O)OH, which subsequently reacts with a —NH₂ terminal on L to create a peptide linkage that links the drug molecule to the rest of general formula (I): X—C(═O)—NH-L-. It will be appreciated, however, that no modification to the therapeutic/drug molecule may be required/necessary if a carboxylic acid is already present in the therapeutic/drug molecule.

In various embodiments, the bioactive moiety is or has been modified to contain one carboxylic acid terminal group. For example, if a carboxylic acid terminal group is absent in a carbohydrate or therapeutic/drug molecule, the carbohydrate or therapeutic/drug molecule may be modified to add a carboxylic acid at one of the carbohydrate or therapeutic/drug molecule terminals. The modification may comprise oxidation reaction(s) to convert a hydroxy group in the carbohydrate to carboxylic acid.

In various embodiments, the repeating unit represented by general formula (I) is in an amount of from about 1 molar % to about 100 molar %, from about 2 molar % to about 99 molar %, from about 3 molar % to about 98 molar %, from about 4 molar % to about 97 molar %, from about 5 molar % to about 96 molar %, from about 10 molar % to about 95 molar %, from about 15 molar % to about 90 molar %, from about 20 molar % to about 85 molar %, from about 25 molar % to about 80 molar %, from about 30 molar % to about 75 molar %, from about 35 molar % to about 70 molar %, from about 40 molar % to about 65 molar %, from about 45 molar % to about 60 molar %, or from about 50 molar % to about 55 molar % relative to the copolymer. In various embodiments, the repeating unit represented by general formula (I) is in an amount of from about 1 molar % to about 10 molar % relative to the copolymer. In various embodiments, the bioactive moiety is about 2 molar %, about 3 molar %, about 4 molar %, about 5 molar %, about 6 molar %, about 7 molar %, about 8 molar %, about 9 molar % or about 10 molar % of the bioactive synthetic copolymer.

In various embodiments, R² is selected from C₁-C₂₀ alkyl, C₂-C₂₀ alkenyl, C₂-C₂₀ alkynyl, C₁-C₂₀ alkoxyalkyl, C₂-C₂₀ alkylcarbonyl or C₃-C₂₀ alkylcarbonylalkyl. The C₁-C₂₀ alkyl substituents may be straight or branched substituents selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl or the like.

In various embodiments, Y¹ is represented by general formula (III):

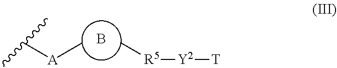

wherein

A is selected from a single bond, oxy, carbonyl, oxycarbonyl, carboxyl, optionally substituted alkoxy, optionally substituted alkoxyalkyl, optionally substituted alkylcarbonyl, optionally substituted alkylcarbonylalkyl, optionally substituted carboxyalkyl, optionally substituted oxycarbonylalkyl, optionally substituted alkylcarboxylalkyl, or optionally substituted alkoxycarbonylalkyl;

B is optionally present as a ring selected from 1,2,3-triazole or succinimide;

R⁵ is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

Y² is selected from the group consisting of polypropylene (PP), polyesters, poly(lactic acid) (PLA), poly(lactic-co-glycolic acid) (PLGA), poly(caprolactone) (PCL), polystyrene (PS), polyacrylates, poly(meth)acrylates, polyamides (PA) and parts thereof; and

T is a terminal group selected from the group consisting of hydrogen, halogen, hydroxyl, amino, acyl, thiol, optionally substituted alkyl, optionally substituted alkenyl, optionally substituted alkynyl, optionally substituted alkoxy, optionally substituted alkoxyalkyl, optionally substituted alkylcarbonyl, optionally substituted alkylcarbonylalkyl, optionally substituted carboxyalkyl, optionally substituted oxycarbonylalkyl, optionally substituted alkylcarboxylalkyl and optionally substituted alkoxycarbonylalkyl.

In various embodiments, Y² is a polyacrylate comprising one or more monomers selected from the group consisting of methyl acrylate, ethyl acrylate, n-propyl acrylate, isopropyl acrylate, n-butyl acrylate, isobutyl acrylate, tert-butyl acrylate, hexyl acrylate, cyclohexyl acrylate, 2-ethylhexyl acrylate, benzyl acrylate and phenyl acrylate. Y² may be poly(methyl acrylate), poly(ethyl acrylate), poly(butyl acrylate) or poly (2-ethylhexyl acrylate). In various embodiments, Y² is a poly(meth)acrylate comprising one or more monomers selected from the group consisting of methyl methacrylate, ethyl methacrylate, n-propyl methacrylate, isopropyl methacrylate, n-butyl methacrylate, isobutyl methacrylate, tert-butyl methacrylate, hexyl methacrylate, cyclohexyl methacrylate, 2-ethylhexyl methacrylate, benzyl methacrylate and phenyl methacrylate. Y² may be poly(methyl methacrylate) (PMMA), poly(ethyl methacrylate) and poly(butyl methacrylate) or poly (2-ethylhexyl acrylate).

In various embodiments, A is selected from a single bond, oxy, carbonyl or oxycarbonylalkyl. A may be a single bond, O, C(═O) and O—C(═O)—R, wherein R is optionally substituted alkyl, optionally substituted alkenyl or optionally substituted alkynyl. In various embodiments, R is straight or branched alkyl substituents selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl or the like. In various embodiments, A is selected from a single bond, O, C(═O) or O—C(═O)—C₁-C₆ alkyl.

In various embodiments, B is absent. In various embodiments, B is present as a ring selected from 1,2,3-triazole or succinimide. Advantageously, 1,2,3-triazole is suitable for connectivity with the present system because of the chemistry used. For example, azide-alkyne click chemistry forms 1,2,3-triazole, which links the norbornene dicarboximide to synthetic polymer Y². Advantageously, succinimide is suitable for connectivity with the present system because of the chemistry used. For example, maleic acid anhydride addition on vinyl-terminated polyolefin forms succinimic acid anhydride, which then reacts with an amine terminal created on norbornene dicarboximide (via hexamethylenediamine (HMDA) or similar diamines) to form succinimide, which links the norbornene dicarboximide to synthetic polymer Y².

In various embodiments, R⁵ is selected from a single bond, optionally substituted alkyl, optionally substituted alkenyl or optionally substituted alkynyl. R⁵ may be a single bond or straight or branched alkenyl substituents selected from ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl or the like. In various embodiments, R⁵ is selected from a single bond or C₂-C₆ alkenyl.

In various embodiments, Y² is selected from the group consisting of polypropylene (PP), polyesters, poly(lactic acid) (PLA), poly(lactic-co-glycolic acid) (PLGA), poly(caprolactone) (PCL), polystyrene (PS), polyacrylates, poly(meth)acrylates, polyamides (PA), and parts thereof. In various embodiments, Y² comprises one or more of the following properties: bioresorbable; inert; long shelf life; mechanical strength; impact resistant; thermal stability; elasticity; elastic recovery; smoothness; biodegradable; lightweight; and low or non-toxicity.

In various embodiments, Y² is substantially devoid of polyalkylene glycol such as polyethylene glycol.

In various embodiments, T is a terminal group selected from the group consisting of hydrogen, halogen, hydroxyl, optionally substituted alkyl, optionally substituted alkenyl, optionally substituted alkynyl, optionally substituted alkylcarboxylalkyl and optionally substituted alkoxycarbonylalkyl. T may be H, OH, halogen selected from Cl, F, Br, I, C₁-C₆ alkyl, C₁-C₆ alkyl-C(═O)—O—C₁-C₆ alkyl or C₁-C₆ alkyl-O— C(═O)—C₁-C₆ alkyl.

In various embodiments, Y¹ is selected from the following general formulae (IIIa), (IIIb), (IIIc), (IIId), (IIIe) or (IIIf), wherein n 1, and m 1:

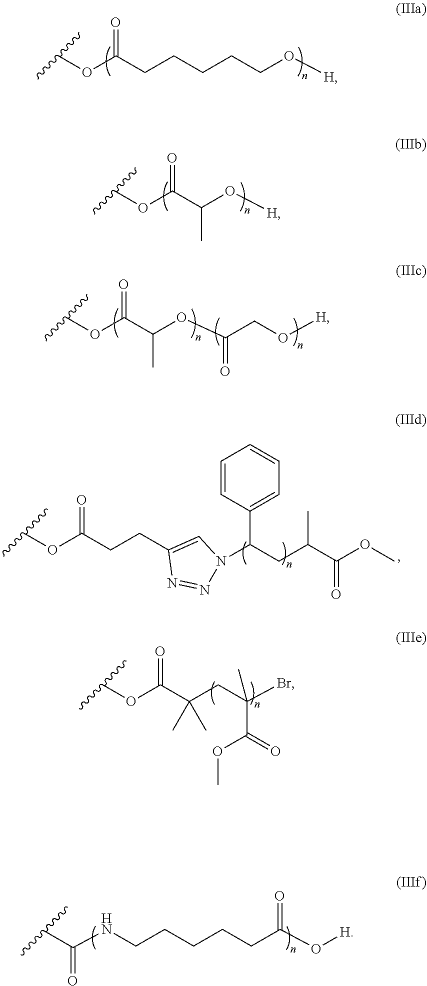

In various embodiments, the total molecular weight of general formula (II) is kept to no more than about 15,000 or no more than about 10,000. It will be appreciated that copolymerisation may become inefficient when the total molecular weight of general formula (I) and (II) is too high. In various embodiments, when the bioactive synthetic copolymer is used for applications which require fast biodegradation, the molecular weight of general formula (II) is kept low by adjusting the value of n and/or m.

In various embodiments, R²—Y¹ is selected from the following, wherein n≥1, and m≥1:

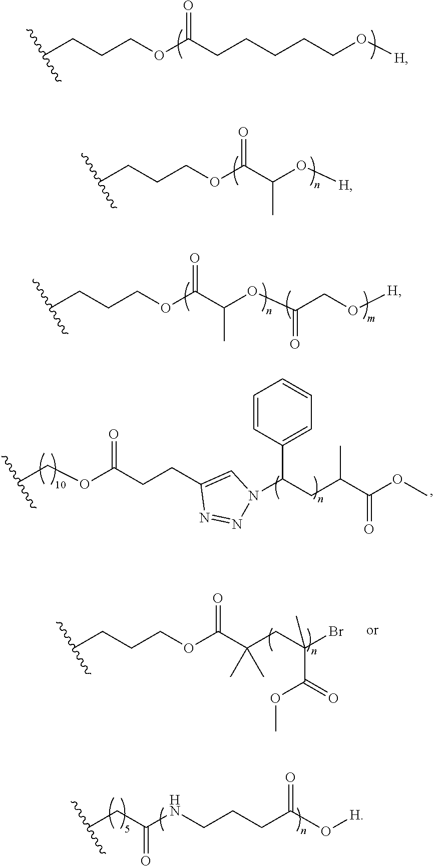

In various embodiments, the ratio of the number of repeating units represented by general formula (I) to the number of repeating units represented by general formula (II) in the bioactive synthetic copolymer is from about 1:1 to about 1:100, from about 1:2 to about 1:99, from about 1:3 to about 1:98, from about 1:4 to about 1:97, from about 1:5 to about 1:96, from about 1:6 to about 1:95, from about 1:7 to about 1:90, from about 1:8 to about 1:85, from about 1:9 to about 1:80, from about 1:10 to about 1:75, from about 1:15 to about 1:70, from about 1:20 to about 1:65, from about 1:25 to about 1:60, from about 1:30 to about 1:55, from about 1:35 to about 1:50, or from about 1:40 to about 1:45. In various embodiments, the ratio of the number of repeating units represented by general formula (I) to the number of repeating units represented by general formula (II) in the bioactive synthetic copolymer is about 1:10, about 1:15, about 1:20, about 1:25, about 1:30, about 1:35, about 1:40, about 1:45 or about 1:50.

In various embodiments, the number of repeating units represented by general formula (I) in the copolymer is from about 10 to about 1,000. In various embodiments, the number of repeating units represented by general formula (II) in the copolymer is from about 10 to about 1,000. In various embodiments, for bone scaffold construction, PLA side chains comprise from about 50 to about 60 lactide units.

In various embodiments, the bioactive synthetic copolymer has a number average molecular weight (Mₙ) of from about 1,000 to about 300,000, 2,000 to about 250,000, from about 3,000 to about 200,000, from about 4,000 to about 150,000, from about 5,000 to about 100,000, from about 10,000 to about 90,000, from about 20,000 to about 80,000, from about 30,000 to about 70,000, from about 40,000 to about 60,000, or about 50,000.

In various embodiments, the bioactive synthetic copolymer has a polydispersity index (PDI) of from about 1.0 to about 10.0. In various embodiments, PDI of the bioactive synthetic copolymer is about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In various embodiments, the bioactive synthetic copolymer has a polydispersity index (PDI) of from about 1.0 to about 3.0, from about 1.05 to about 2.95, from about 1.1 to about 2.9, from about 1.2 to about 2.8, from about 1.4 to about 2.6, from about 1.6 to about 2.4, from about 1.8 to about 2.2 or about 2.0. In various embodiments, the PDI of the bioactive synthetic copolymer is no more than 1.50.

In various embodiments, the one or more repeating units represented by general formula (I) and the one or more repeating units represented by general formula (II) are designed to link to the poly(norbornene) backbone via at least covalent interactions. In various embodiments, each repeating unit represented by general formula (I) is covalently bonded to the poly(norbornene) backbone and/or each repeating unit represented by general formula (II) is covalently bonded to the poly(norbornene) backbone. Advantageously, as bioactive moieties (in general formula (I)) are covalently bonded to the bioactive synthetic polymer, bioactivity is localized. In various embodiments, the bioactive moieties such as biomolecules do not leach out from the polymer, therefore preventing undesirable/unwanted side effects caused by biomolecules entering the circulatory system and/or reaching unintended parts of the body system. Embodiments of the bioactive synthetic copolymer therefore overcome problems faced by conventional biomolecules that are administered as drugs which may metabolized prematurely before therapeutic effects are achieved. In various embodiments, the bioactive moieties such as drug molecules do not leach out into media which can escape into the environment in the event that disposal is improperly managed.

It will be appreciated that other interactions such as Van der Waals interactions may also be present within the copolymer.

In various embodiments, the bioactive synthetic copolymer comprises a brush, bottlebrush, block, comb or graft-copolymer structure. In various embodiments, the repeating units may be randomly distributed/arranged within the polymer.

In various embodiments, the one or more repeating units represented by general formula (I) comprises two or more different types of bioactive moiety X. In various embodiments, the one or more repeating units represented by general formula (I) comprises 2, 3, 4, 5, 6, 7 or 8 different types of bioactive moiety X. For example, within a bioactive synthetic copolymer, there may be repeating units represented by general formula (I) comprising peptide as X and repeating units represented by general formula (I) comprising carbohydrate as X. Advantageously, in various embodiments, the bioactive synthetic copolymer imparts two or more different types of bioactivities.

In various embodiments, the one or more repeating units represented by general formula (II) comprises two or more different types of synthetic polymer Y². In various embodiments, the one or more repeating units represented by general formula (II) comprises 2, 3, 4, 5, 6, 7 or 8 different types of synthetic polymer Y².

In various embodiments, the bioactive synthetic copolymer is a random polymer or a block copolymer. In some embodiments, the block polymer is a di-block or a triblock polymer. For example, the copolymer may have or is made up of two or three different polymer blocks. In some embodiments, the multi-block copolymer comprises more than three polymeric blocks. The blocks may be randomly distributed/arranged within the polymer.

In various embodiments, the bioactive synthetic copolymer is selected from one of the following: PCL-(GPHyp)₃ copolymer comprising (GPHyp)₃ in general formula (I) and PCL in general formula (II); PA-DGEA copolymer comprising DGEA in general formula (I) and PA in general formula (II); PS-ciprofloxacine copolymer comprising ciprofloxacine in general formula (I) and PS in general formula (II); PLA-RGD copolymer comprising RGD in general formula (I) and PLA in general formula (II); PLGA-(GPHyp)₃ copolymer comprising (GPHyp)₃ in general formula (I) and PLGA in general formula (II); and PMMA-(GPHyp)₃ copolymer comprising (GPHyp)₃ in general formula (I) and PMMA in general formula (II).

Advantageously, the bioactive synthetic copolymer disclosed herein is highly customizable. Depending on the application that the bioactive synthetic copolymer is intended, X with the desired biological activity and Y² with the desired physical attributes may be selected to eventually obtain the bioactive synthetic copolymer with the desired repeating units represented by general formulae (I) and (II).

In various embodiments, the bioactive synthetic copolymer is blended with a base polymer for further use. In various embodiments, the base polymer is similar to or of the same type as the synthetic polymer Y² used in general formula (II). In various embodiments, a medical grade polymer is used for base material while low molecular weight synthetic polymer is used in the synthetic side chain of the bioactive synthetic copolymer. Advantageously, embodiments of the bioactive synthetic polymer allow for biomolecule to be blended into base material of synthetic polymer similar to the synthetic polymer side arms of copolymer, without phase separation.

Methods

There is provided a method of preparing a bioactive synthetic copolymer, the method comprising: polymerizing one or more bioactive macromolecules represented by general formula (IV) with one or more synthetic macromolecules represented by general formula (V) to obtain the bioactive synthetic copolymer:

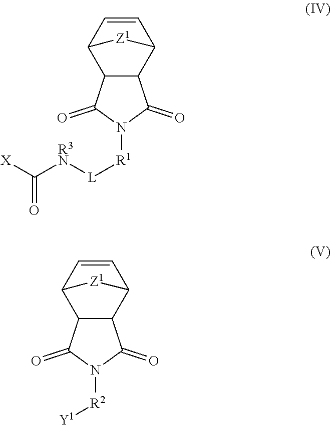

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y¹ comprises a synthetic polymer; and

Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

Advantageously, in various embodiments, the method of preparing a bioactive synthetic copolymer as disclosed herein is also a modular method for designing a bioactive synthetic copolymer.

There is also provided a modular method of designing a bioactive synthetic copolymer, the method comprising: selecting one or more macromolecules from a first module based on desired biological activity, the first module consisting of a library of norbornene-dicarboximide-containing bioactive macromolecules represented by general formula (IV) with known biological activities; selecting one or more macromolecules from a second module based on desired physical attributes, the second module consisting of a library of norbornene-dicarboximide-containing synthetic macromolecules represented by general formula (V) with known physical attributes; and polymerizing the one or more macromolecules selected from the first module with the one or more macromolecules selected from the second module to obtain the bioactive synthetic copolymer:

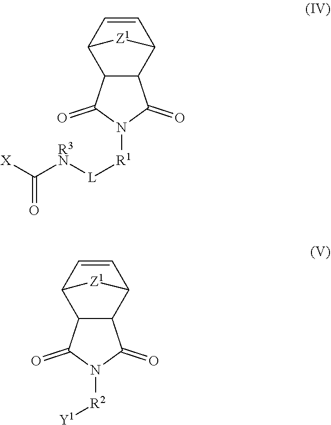

Advantageously, the methods disclosed herein allow rapid customization and quick development/construction of the bioactive synthetic copolymer with the desired bioactivity and physical properties.

In various embodiments, the polymerization reaction comprises one or more olefin metathesis chain-growth polymerization step(s). The olefin metathesis chain-growth polymerization may be ring opening metathesis polymerization (ROMP). In various embodiments, the ROMP reaction occurs at the reactive moiety of the macromonomers, for e.g., at the olefins/alkene/C═C moieties. The ROMP may comprise a number of different approaches, including “arm-first” ROMP, “brush-first” ROMP, “graft-to” ROMP, “graft-from” ROMP, “graft-through” ROMP, or combinations thereof. Advantageously, ROMP allows quick development/construction of well-defined synthetic polymers with the desired bioactivities. In various embodiments, depending on the targeted application, an appropriate synthetic polymer and a biomolecule with the bioactivity of interest may be chosen and copolymerized together using ROMP.

In various embodiments, the polymerization reaction is performed in the presence of a polymerisation initiator/catalyst/promoter. In various embodiments, the polymerisation initiator/catalyst/promoter comprises a metal complex. The metal complex may be a ruthenium (Ru), molybdenum (Mo) or tungsten (W) complex. In various embodiments, ROMP is performed in the presence of a ruthenium complex. Advantageously, as compared to other transition metals (e.g., W and Mo), Ru is more stable in the presence of polar functional groups, thereby making Ru a suitable olefin metathesis catalyst for ROMP reactions that involve bioactive moieties selected from the group consisting of proteins, peptides, carbohydrates, therapeutic/drug molecules and derivatives thereof. In various embodiments, Ru is air-stable (i.e. stable in air) and thermally stable (i.e. stable at high temperatures) whilst being commercially available on a large scale, allowing ROMP to be carried out at elevated temperatures. The ruthenium complex may comprise a Grubbs catalyst selected from a first-generation Grubbs catalyst, second-generation Grubbs catalyst, Hoveyda-Grubbs' catalyst, a third-generation Grubbs catalyst or derivatives thereof.

In various embodiments, R¹, R², R³, L, X, Y¹, Z¹ and Z² contain one or more features and/or share one or more properties that are similar to those described above.

In various embodiments, the polymerization reaction comprises a) mixing one or more bioactive macromolecules represented by general formula (IV) with one or more synthetic macromolecules represented by general formula (V) to obtain a solution; b) adding the catalyst to the solution from a); and c) precipitating the bioactive synthetic copolymer.

In various embodiments, step a) and/or step b) is/are carried out or undertaken at a temperature in the range of from about 20° C. to about 100° C. The temperature(s) at which step a) and step b) is carried out may be independently selected from a temperature of about 20° C., about 25° C., about 30° C., about 35° C., about 40° C., about 50° C., about 60° C., about 70° C., about 80° C., about 90° C. or about 100° C.

In various embodiments, step a) and/or step b) is/are carried out or undertaken for a time period in the range of from about 30 mins to about 3 days. The time period at which step a) and step b) is carried out may be independently selected from a time period of about 30 mins, 1 hour, 2 hours, 3 hours, 4 hours, 5 hours, 10 hours, 20 hours, 1 day, 2 days or 3 days.

In various embodiments, step a) and/or step b) is/are carried out in the presence of an organic solvent. The organic solvent may be a protic solvent, an aprotic solvent or combinations thereof. In various embodiments, the organic solvent(s) for step a) and step b) is independently selected from the group consisting of tetrahydrofuran (THF), benzene, toluene, acetonitrile (ACN), dichloromethane (DCM), dimethyl sulfoxide (DMSO), acetone, methyl ethyl ketone (MEK), formic acid, acetic acid and the like and combinations thereof. In various embodiments, protic solvents such as formic acid and/or acetic acid may be used especially for PA-based materials (e.g. when Y¹ comprises formula IIIf). In various embodiments, the organic solvent used is the same for step a) and b). It is to be appreciated that the type of solvent used is dependent on the type of reactants used and is not limited to the above.

In various embodiments, step c) is/are carried out in a mixture of organic solvents. The mixture of organic solvents may contain one or more aprotic organic solvents and one or more protic organic solvents. In various embodiments, the mixture of organic solvents for step c) is selected from the group consisting of tetrahydrofuran (THF), benzene, toluene, acetonitrile (ACN), dichloromethane (DCM), dimethyl sulfoxide (DMSO), acetone, methyl ethyl ketone (MEK), ethyl vinyl ether, methanol, ethanol, butanol and the like and combinations thereof. It is to be appreciated that the type of solvent used is dependent on the type of reactants used and is not limited to the above.

Advantageously, by carrying out polymerization with the carefully designed/controlled conditions described above, embodiments of the method disclosed herein have successfully overcome the widely varying and/or opposing properties of the individual components (e.g., L, X, Y components) to construct the bioactive synthetic copolymer disclosed herein.

There is also provided a method of preparing a bioactive homopolymer, the method comprising: polymerising one or more bioactive macromolecules represented by general formula (IV) to obtain the bioactive homopolymer:

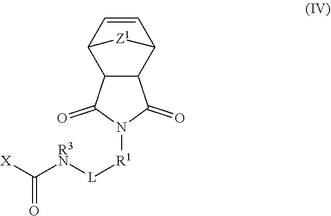

wherein R¹ is optionally substituted alkyl; R³ is selected from H, optionally substituted alkyl, optionally substituted alkenyl or optionally substituted alkynyl; L is heteroalkylene; X comprises a bioactive moiety selected from the group consisting of proteins, peptides, carbohydrates, therapeutic/drug molecules and derivatives thereof; and Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

There is also provided a method of preparing a synthetic homopolymer, the method comprising: polymerising one or more synthetic macromolecules represented by general formula (V) to obtain the synthetic homopolymer:

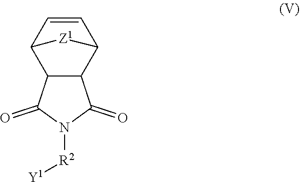

wherein R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl; Y¹ comprises a synthetic polymer; and Z² is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

### Bioactive Macromolecule

There is also provided a bioactive macromolecule represented by general formula (IV) for preparing the copolymer disclosed herein:

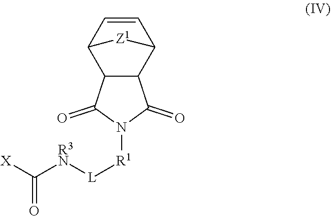

wherein

R¹ is optionally substituted alkyl;

R³ is selected from H, optionally substituted alkyl, optionally substituted alkenyl or optionally substituted alkynyl;

L is heteroalkylene;

X comprises a bioactive moiety selected from proteins, peptides, carbohydrates, therapeutic/drug molecules or derivatives thereof; and

Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

In various embodiments, R¹, R³, L, X and Z¹ contain one or more features and/or share one or more properties that are similar to those already described above.

In various embodiments, the bioactive macromolecule undergoes self-polymerization or co-polymerization. In various embodiments thereof, the bioactive macromolecule also behaves as a bioactive macromonomer.

In various embodiments, X is coupled to the norbornene dicarboximide through a carboxylic acid functionality in the following arrangement: —R¹-L-NR³—C(═O)—X. Advantageously, by linking X through a carboxylic acid functionality, amine terminal group(s) in X is/are free up for delivering its bioactivity, therefore ensuring the bioavailability of X. It will be appreciated that as amine group(s) confer bioactivity, exhausting up amine groups in bioactive moieties for polymer binding may be undesirable.

In various embodiments, X is coupled to the norbornene dicarboximide via peptide/amide linkage, i.e. —NR³—C(═O)—. Advantageously, the bioactive macromolecule disclosed herein is considerably stronger and/or stable than conventional macromolecules that contain ester linkages. Without being bound by theory, it is believed that amide linkages are stronger than ester linkages because ester linkages are more prone to hydrolysis, which may release bioactive moieties into the bloodstream, leading to a premature metabolism of bioactive moieties.

There is also provided a method of preparing a bioactive macromolecule disclosed herein, the method comprising: (i) providing a dicarboxylic anhydride having general formula (VI):

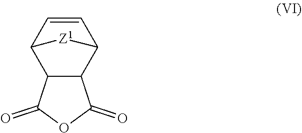

wherein Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl;
(ii) reacting said dicarboxylic anhydride having general formula (VI) with a diamine R⁴R³N-L-R¹—NH₂ to obtain an amine having general formula (VII):

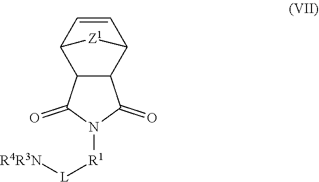

wherein R¹ is optionally substituted alkyl; R³ and R⁴ are each independently selected from H, optionally substituted alkyl, optionally substituted alkenyl or optionally substituted alkynyl, wherein at least one of R³ and R⁴ is H; L is heteroalkylene; Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl; and
(iii) reacting said amine having general formula (VII) with an acid-containing bioactive moiety X—C(═O)OH to obtain the bioactive macromolecule, wherein X comprises a bioactive moiety selected from the group consisting of proteins, peptides, carbohydrates, therapeutic/drug molecules and derivatives thereof.

In various embodiments, R¹, R³, L, X and Z¹ contain one or more features and/or share one or more properties that are similar to those described above.

In various embodiments, R³ and R⁴ are each independently selected from H, C₁-C₂₀ alkyl, C₂-C₂₀ alkenyl or C₂-C₂₀ alkynyl, wherein at least one of R³ and R⁴ is H.

In various embodiments, step (ii) comprises the use of diamine R⁴R³N-L-R¹—NH₂ for coupling X to norbornene dicarboxylic anhydride. The diamine used may be one that is commercially available. Advantageously, the method is a straightforward reaction and does not require a poly(ethylene glycol) aminocarboxylic acid which is commercially unavailable and synthetically challenging to make. In various embodiments therefore, the method does not require tedious multi-step and/or low yielding synthesis procedures. In various embodiments, the diamine R⁴R³N-L-R¹—NH₂ is used in slight excess to ensure that the amine having general formula (VII) does not become connected with norbornene dicarboximide on two ends, which would otherwise turn the diamine R⁴R³N-L-R¹—NH₂ into a linker instead of a terminating group.

In various embodiments, the diamine is a heteroalkylene diamine, wherein L is heteroalkylene. In various embodiments, L is C₂₀-C₃₀₀ heteroalkylene or a heteroalkylene having from 20 carbon atoms to 300 carbon atoms. In various embodiments, L has a number average molecular weight of between about 500 and about 7,000. In various embodiments, the heteroatom in L is O. In various embodiments, L is polyalkylene glycol. In various embodiments, the diamine is a poly(ethylene glycol) diamine, wherein L is poly(ethylene glycol). In various embodiments, L is selected from the group consisting of PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₂₀₀₀, PEG₂₅₀₀, PEG₃₀₀₀, PEG₃₅₀₀, PEG₄₀₀₀, PEG₄₅₀₀, PEG₅₀₀₀, PEG₆₀₀₀ and mixtures thereof.

In various embodiments, the method further comprises, prior to step (iii), purifying the amine having general formula (VII) to isolate the product and/or remove impurities. In various embodiments, the step of purifying comprises washing with at least one of an acid or a base. The step of purifying may comprise washing with at least one of an acid or a base at least once, at least twice, at least thrice, at least four times, at least five times, at least six times, at least seven times or at least eight times to neutralise the amine having general formula (VII). In various embodiments, the step of purifying comprises double neutralisation steps. In one embodiment, the double neutralisation comprises a first step of washing with acid to remove unreacted diamine R⁴R³N-L-R¹—NH₂ and a second step of washing with base to neutralise the amine having general formula (VII). It will be appreciated that as the diamine R⁴R³N-L-R¹—NH₂ is basic, adding acid to said diamine will neutralise the diamine for removal from the amine having general formula (VII). It will also be appreciated that although the first step of washing with acid may protonate the amine having general formula (VII) at the amine terminal, the subsequent second step of washing with base or excess base converts the protonated form back into its free amine form. The acid used for the first neutralisation step may be selected from the group consisting of HCl, HNO₃, H₂SO₄ and H₃PO₄. The base used for the second neutralisation step may be selected from the group consisting of NaOH, KOH, NH₄OH and Ca(OH)₂. In various embodiments, the second neutralisation step comprises washing with base at least once, at least twice, at least thrice or at least four times to fully extract the amine having general formula (VII) for maximised yield. In one embodiment, the second neutralisation comprises washing with base twice. Without being bound by theory, it is believed that up to 30% of the protonated form of amine having general formula (VII) may reside in the aqueous phase during extraction. In various embodiments therefore, the step of washing with base comprises washing the aqueous phase once with base and washing the organic phase once with base in order to completely extract the amine having general formula (VII) from both the aqueous and organic phases. Advantageously, by using double neutralisation steps after coupling to obtain the free amine terminus, the method eliminates the need for any additional steps such as protection/deprotection step(s). It will be appreciated by a person skilled in the art that the use of diamine, particularly poly(ethyleneglycol) diamine is extremely challenging and typically requires protection of one amine terminal in order to couple to a norbornene dicarboxylic acid anhydride. Indeed, in various embodiments, the polyalkylene glycol such as PEG are used as spacers, linkers or linking groups in the overall polymers, instead of as terminal groups. Thus, it may appear intuitive to consider protecting one amine terminal of a PEG diamine to couple it with norbornene dicarboxylic acid anhydride. The protecting group may then be removed to expose the amine terminus for further reactions. However, this would add extra steps to the reaction and hence may not be desirable. Embodiments of the present disclosure has managed to overcome this problem in the synthesis and purification steps by carrying out double neutralization steps after coupling to obtain the free amine terminus for further coupling to peptides.

### Material Comprising Bioactive Synthetic Copolymer

There is also provided a material comprising a copolymer as disclosed herein for use in medicine. In various embodiments, the material is part of or used on an apparatus selected from the group consisting of wound dressing, skin scaffold, bone scaffold, organoid scaffolds, implants, medical devices. For example, the material may be a scaffold for tissue regeneration comprising the bioactive synthetic copolymer disclosed herein. The material may be a material suitable for increasing biocompatibility of polyamide used in medical devices through stimulation of collagen. The material may be an antibacterial polystyrene material suitable for use in tissue and serum handling devices. The material may be a polylactide scaffold suitable for stimulating tissue regeneration. The material may be a poly(lactic-co-glycolic acid) scaffold suitable for stimulating cartilage tissue regeneration. The material may also a poly(methyl methacrylate) material for use in medical implants.

In various embodiments, the material is processed/printed/three-dimensionally printed via electrospinning, melt extrusion, hot melt extrusion, injection moulding, fused filament fabrication, fused deposition modelling, additive manufacturing, melt blowing and the like.

In various embodiments, the material or bioactive synthetic copolymer is compatible with biological systems or parts of the biological systems without substantially or significantly eliciting an adverse physiological response such as a toxic reaction/response, an immune reaction/response, an injury or the like when used on/in the human or animal body. In various embodiments, the polymer is substantially devoid of materials that elicit an adverse physiological response.

There is also provided a method of accelerating/stimulating/promoting cell growth or tissue regeneration such as bone tissue or skin tissue regeneration, or wound healing, the method comprising administering/applying the bioactive copolymer or material disclosed herein to a human or animal body.

There is also provided use of the bioactive synthetic copolymer or material disclosed herein in the manufacture of a medicament for accelerating/stimulating/promoting cell growth or tissue regeneration, or wound healing such as bone tissue or skin tissue regeneration.

There is also provided use of the bioactive synthetic copolymer or material disclosed herein for biofilm eradication.

In various embodiments, the bioactive synthetic copolymer is substantially devoid of stem cells and/or growth factor. In various embodiments, the bioactive synthetic copolymer is non-biofouling.

In various embodiments, the bioactive moiety is directly chemically linked to the copolymer. In various embodiments, the bioactive moiety is not being encapsulated in the polymer matrix.

In various embodiments, the bioactive moiety (for e.g., peptide) is not connected to the norbornene dicarboximide via an amino butyric acid spacer.

In various embodiments, the bioactive moiety comprises structurally well-defined collagen with specific sequences. In various embodiments, the bioactive moiety is substantially devoid of animal derived collagen which have broad molecular weight distributions and/or ill-defined structures and/or known to elicit negative immune response in human body.

In various embodiments, polyethylene glycol is not used as a monomer on its own. For example, in various embodiments ethylene glycol units are not present in the copolymer/macromolecule as terminal groups.

Embodiments of the bioactive synthetic polymer and/or methods disclosed herein do not involve any release of bioactive molecule such as drug molecule from the copolymer on activation methods such as photoactivation. Embodiments of the bioactive synthetic polymer is substantially devoid of a photocleavable group.

### BRIEF DESCRIPTION OF FIGURES

FIG. 1 is a schematic diagram 100 of a bioactive synthetic polymer in accordance with various embodiments disclosed herein.

FIG. 2 shows the thermogravimetric analysis (TGA) graphs of pure RGD peptide (“RGD(PURE)”), NBPEG₃₄₀₀RGD macromonomer (“NB-PEG3400 RGD”), NBPCL macromonomer (“NB-PCL”) and PCL-RGD ROMP copolymer (“PCL_PEG3400_RGD”).

FIG. 3 is a graph showing the biocompatibility of PCL-peptide based materials prepared in accordance with various embodiments disclosed herein, relative to a control. The results were obtained from cell viability tests of human fibroblasts (Hs27) on PCL-peptide based materials over a period of 72 hours, where macromonomers of 3 peptides (SRGDS, (GPHyp)₃ and DGEA) have been copolymerized with macromonomers of PCL. Comparative example is commercial dressing Allevyn (i.e. polyurethane-based dressing) and Acticoat (Silver nanoparticle-based dressing).

FIG. 4 is a graph showing the BMP-2-induced ALP activity of PCL-peptide based materials after 72 hours. Commercial PCL is used as the control.

FIG. 5 shows the thermogravimetric analysis (TGA) graphs of NBPEG₃₄₀₀(GPHyp)₃ macromonomer (“NB-PEG-GPHP”), PA6 ROMP polymer (“PA6-homopoly”) and PA6-(GPHyp)₃ ROMP copolymer (“PA6-GPHP”).

FIG. 6 is a graph showing the cell proliferation results obtained from cell viability tests of human fibroblasts (Hs27) cultured on polyamide 6 (PA6)-based electrospun sheets using a Luminescent Cell Viability Assay (CellTiter-Glo). The PA-collagen materials are PA6-(PHypG)₃, PA6-(GPHyp)₃ and PA6-DGEA, where both (PHypG)₃ and (GPHyp)₃ are collagen fragments and DGEA is a collagen mimic. Controls used are poly(norbornene dicarboximide) with PA6 side chains; and poly(norbornene dicarboximide) with PA6 and mPEG₅₀₀₀ as side chains. PA6-homopoly refers to poly(norbornene dicarboximide) with PA6 side chains; PA6-mPEG refers to poly(norbornene dicarboximide) with PA6 and mPEG₅₀₀₀ as side chains; PA6-PHPG refers to PA6-(PHypG)₃ copolymer; and PA6-GPHP refers to PA6-(GPHyp)₃ copolymer.

FIG. 7 is a graph showing the cell proliferation results obtained from cell viability tests of human fibroblasts (Hs27) cultured on poly(lactide) (PLA)-based electrospun sheets using a Luminescent Cell Viability Assay (CellTiter-Glo). The bioactive synthetic copolymer is PLA-RGD and commercial base polymer PLA is used as the control (“PLA bulk”).

FIG. 8 is a graph showing the biocompatibility results obtained from cell viability tests of human fibroblasts (Hs27) cultured on poly(lactic-co-glycolic acid) (PLGA)-based electrospun sheets using a Luminescent Cell Viability Assay (CellTiter-Glo). The bioactive synthetic copolymer is PLGA-RGD. Controls used are commercial base polymer PLGA (PLGA-Bulk), poly(norbornene dicarboximide) with PLGA side chains; and poly(norbornene dicarboximide) with PLGA and mPEG₅₀₀₀ as side chains. PLGA-homo refers to poly(norbornene dicarboximide) with PLGA side chains; and PLGA-mPEG refers to poly(norbornene dicarboximide) with PLGA and mPEG₅₀₀₀ as side chains.

FIG. 9 is a graph showing the biocompatibility results obtained from cell viability tests of human fibroblasts (Hs27) cultured on poly(methyl methacrylate) (PMMA)-based electrospun sheets using a Luminescent Cell Viability Assay (CellTiter-Glo). The bioactive synthetic copolymer is PMMA-(GPHyp)₃ (“PMMA-GPHP”). Controls used are commercial base polymer PMMA (PMMA-bulk), poly(norbornene dicarboximide) with PMMA side chains; and poly(norbornene dicarboximide) with PMMA and mPEG₅₀₀₀ as side chains. PMMA-homo refers to poly(norbornene dicarboximide) with PMMA side chains; and PMMA-mPEG refers to poly(norbornene dicarboximide) with PMMA and mPEG₅₀₀₀ as side chains.

### EXAMPLES

Example embodiments of the disclosure will be better understood and readily apparent to one of ordinary skill in the art from the following examples, tables and if applicable, in conjunction with the figures. It should be appreciated that other modifications related to structural, and chemical changes may be made without deviating from the scope of the invention. Example embodiments are not necessarily mutually exclusive as some may be combined with one or more embodiments to form new example embodiments. The example embodiments should not be construed as limiting the scope of the disclosure.

### Example 1: Modular Approach to Constructing Bioactive Synthetic Polymer

A general strategy for construction of bioactive macromonomers containing either peptides, carbohydrates or drug molecules has been developed. The simple 2-step synthesis allows for rapid build up of a wide library of bioactive macromonomers of various chain length, allowing for quick development of synthetic polymers with desired bioactivities as required in targeted application. A modular approach to construct the desired polymers to suit various applications by matching the bioactive macromonomer with macromonomers of synthetic polymers with desired physical properties, is made possible using this library. Rapid polymer customization can thus be achieved.

A modular building block system to designing/constructing desired bioactive materials has been developed as shown in Scheme 1. Once the targeted medical application has been identified, a “plug and play” approach (Scheme 1) can be used to create the desired bioactive synthetic material which not only possess therapeutic effects but also, necessary mechanical properties for easy storage and handling.

By using the modular approach, a macromonomer consisting of a bioactive molecule at the terminal of the monomer is created and can be copolymerized with other synthetic polymers to create bioactive synthetic polymers with targeted bioactivities. The desired polymer is highly customizable using the strategy developed in accordance with various embodiments disclosed herein, by switching the bioactive molecule for any peptide or carbohydrate that bears a carboxylic acid group. This allows for rapid synthesis of bioactive polymers once a target application is identified. The bioactive polymers created can have properties ranging from skin cell regeneration, bone cell regeneration, antimicrobial activity, cartilage tissue regeneration, wound healing, collagen production, anti-inflammatory to cholesterol synthesis inhibition (for e.g., using atorvastatin as drug) and can be made to be mechanically tough or biodegradable, depending on the needs. The modular synthesis therefore makes application matching to polymer properties much simpler and effective.

### Example 2: Method of Preparing Bioactive Synthetic Copolymer

The method of preparing a bioactive synthetic copolymer in accordance with various embodiments disclosed herein involve creating macromonomers of the bioactive molecules and synthetic polymers separately and using ring opening metathesis polymerization (ROMP) techniques to link these otherwise mutually incompatible molecules together. The result is a brush polymer bearing both the bioactive molecule and the synthetic polymer for overall mechanical strength of the material (Scheme 2). By creating the macromonomers separately, the inventors are able to build a library of macromonomers with different properties for clinicians or medtech companies to choose from, and the material with desired therapeutic effects can be constructed easily and rapidly, to suit the targeted application. By creating the macromonomers separately, the inventors are also able to build a library of macromonomers and the eventual copolymers, for rapid testing of efficacy in the biomedical laboratory. Different combinations of these macromonomers (MMs) can also generate a library of well-defined brush copolymers containing different bioactive molecules for rapid screening of bioactivity in laboratory.

In the following examples, brush polymers containing pendant arms of synthetic polymers and bioactive molecules tethered on polyethylene glycol (PEG) moieties, have been created. Synthetic polymers may include poly(caprolactone) (PCL), polyesters such as poly(lactic acid) (PLA) and poly(lactic-co-glycolic acid) (PLGA), polystyrene (PS), polyacrylates, poly(meth)acrylates such as poly(methyl methacrylate) (PMMA) and polyamides (PA). Bioactive molecules may include biomolecules selected from peptide sequences of any combination of the 20 natural amino acids, from 3-20 amino acid residues, carbohydrates such as glycosaminoglycans or drug molecules containing a carboxylic acid terminal such as certain antibiotics. Biomolecules may also include collagen mimic peptides from 3-20 amino acid residues in any sequence, such as DGEA, (Gly-Pro-Hyp)₃ and (Pro-Hyp-Gly)₃. Depending on the application, the appropriate synthetic polymer and biomolecule with the bioactivity of interest can be chosen, copolymerized together using the brush polymer technology as disclosed herein by ring opening metathesis polymerization.

The resultant polymer shows bioactivity of the biomolecule involved while having much better physical and mechanical properties for good material handling and processability. For example, improvement in cell viability or cell proliferation in both the PA-collagen copolymers and PLA-RGD copolymers over controls, were observed.

The polymers can be subsequently blended with polymers similar to that on the pendant arms to create bioactive materials for use in biomedical devices such as catheters, wound dressings, tissue scaffolds, plastic surgery implants, prosthetic parts, cartilage joint implants etc.

The synthetic route for preparing a bioactive synthetic polymer in accordance with various embodiments disclosed herein is illustrated in Scheme 2.

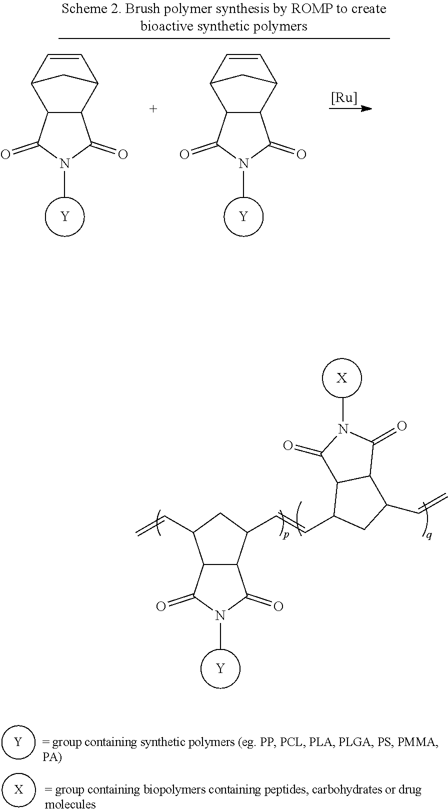

In the following examples, six types of synthetic polymers were selected for the synthetic polymer side chains on the brush polymers. The exact polymer to be selected is dependent on the nature of the biomedical device to be manufactured, for example whether properties such as biodegradability, flexibility, impact-resistance etc, are required in the device material.

### Example 3: Bioactive Macromonomers and Method of Synthesis

A general strategy for the synthesis of bioactive macromonomers in accordance with various embodiments disclosed herein has been developed. Polyethylene glycol diamine of various chain lengths (e.g., Mw=1,000-6,000) are reacted with cis-norbornene-exo-2,3-dicarboxylic anhydride to create the main macromonomer body, i.e. macromonomer body containing norbornene dicarboximide and polyethylene glycol (NBPEG) (Scheme 3.1). Once NBPEG is created, various peptides, carbohydrates or drug molecules are then reacted with these NBPEG chains to create the bioactive macromonomer with desired therapeutic properties.

With the main body of macromonomer, any peptides, carbohydrates or drug molecules (R) can be used using the carboxylic acid terminus on the bioactive molecule by means of condensation reaction to form peptide/amide bonds between the amine group on NBPEG and carboxylic acid terminal of the carbohydrate or peptide (Scheme 3.2). Examples of drug molecules include antibiotics such as amoxicillin or ciprofloxacin. In various embodiments, R is copolymers of antimicrobial peptides (IRIK)₂ or (IKKI)₃; heparin oligosaccharides DP10, DP12, DP14; COL or RGD, where COL may be DGEA, (GPHyp)ₙ or (PHypG)ₙ. In various embodiments, R is carbohydrates or drug molecules with a CO₂H group or peptide sequences of 3-20 amino acid residues, formed from 20 natural amino acids. In various embodiments, R is DP12, DP14, COL or RGD, where COL may be DGEA or (GPHyp)ₙ. Should the carbohydrate not possess a carboxylic acid group, modification of the carbohydrate to include one may be necessary. Alternatively, amine substitution reactions or reductive amination reactions can also be done on the carbohydrate's hydroxy or carbonyl groups.

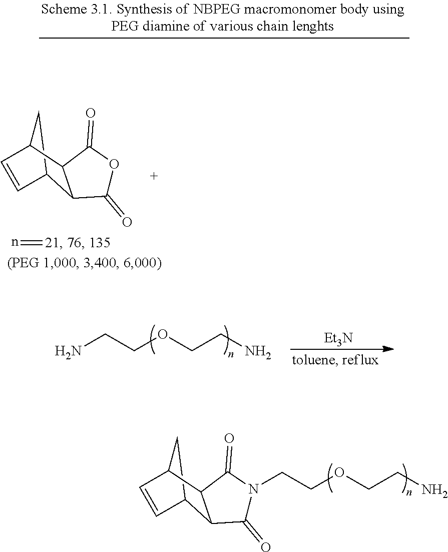

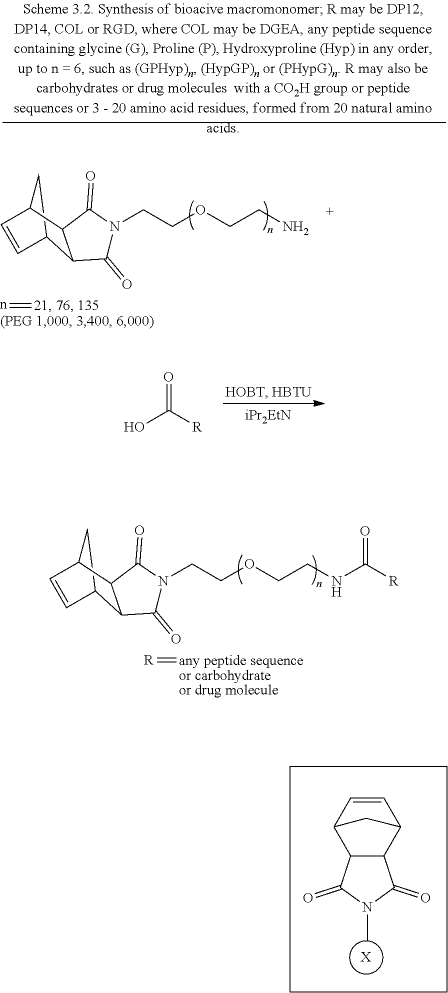

### Example 4: Synthetic Macromonomers and Method of Synthesis

Schemes 4.1 to 4.5 show synthetic macromonomers of PCL, PLA, PLGA, PS, PMMA and PA.

PLA, PLGA, PCL are created using ring opening polymerization on a norbornene dicarboximide linker with a terminal hydroxy group. Briefly, cis-norbornene-exo-2,3-dicarboxylic anhydride is reacted with 3-amino-1-propanol to create an initiator molecule. This initiator is then reacted with ε-caprolactone (or D, L-lactide for PLA formation; D, L-lactide and glycolide for PLGA formation) in the presence of Sn(Oct)₂ catalyst to form PCL chains on the norbornene dicarboximide linker, N-[3-hydroxylpropyl]-cis-5-norbornene-exo-2,3-dicarboximide (NPH), to give the PCL macromonomer (NB-PCL) (Scheme 4.1) (or NB-PLA macromonomer).

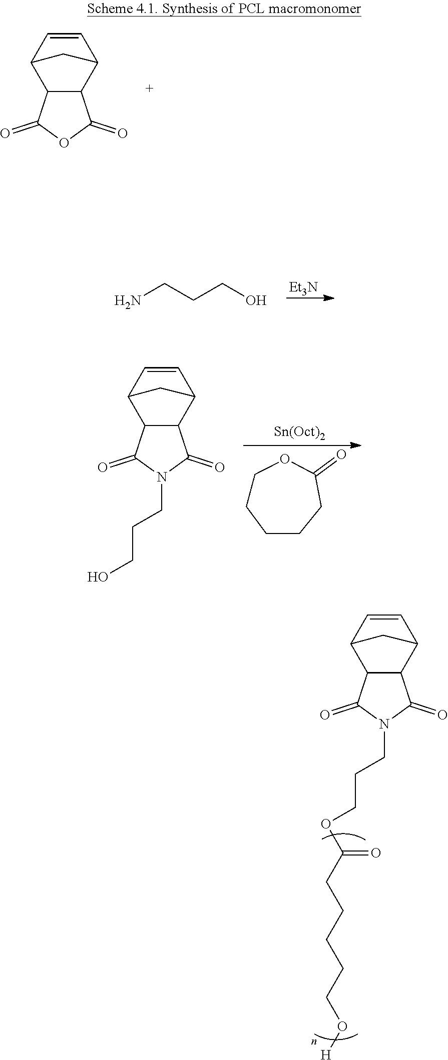

PLA macromonomer is synthesized using ring-opening polymerization. Cis-norbornene-exo-2,3-dicarboxylic anhydride is first reacted with 3-amino-1-propanol to provide the initiator molecule. This alcohol initiator is then stirred with D, L-lactide in the presence of Sn(Oct)₂ catalyst to provide NB-PLA macromonomer (Scheme 4.2).

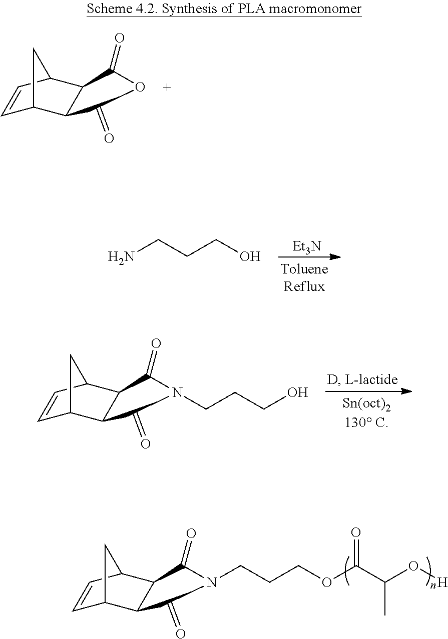

Poly(lactic-co-glycolic acid) (PLGA) macromonomer is synthesized in 2 steps via a cis-norbornene-exo-2,3-dicarboximide aminopropanol initiator molecule (Scheme 4.3).

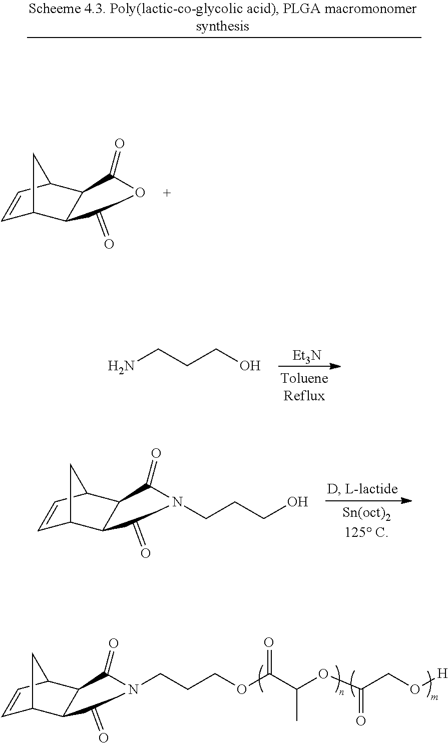

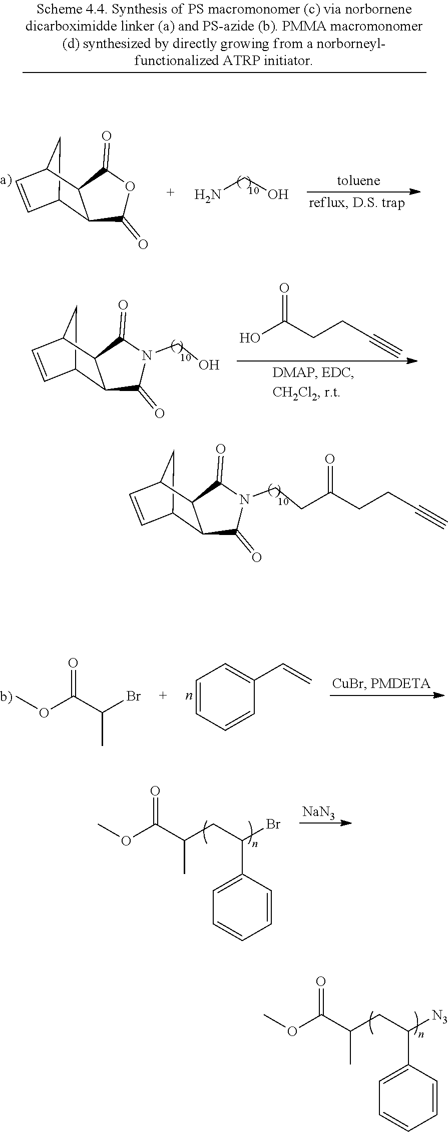

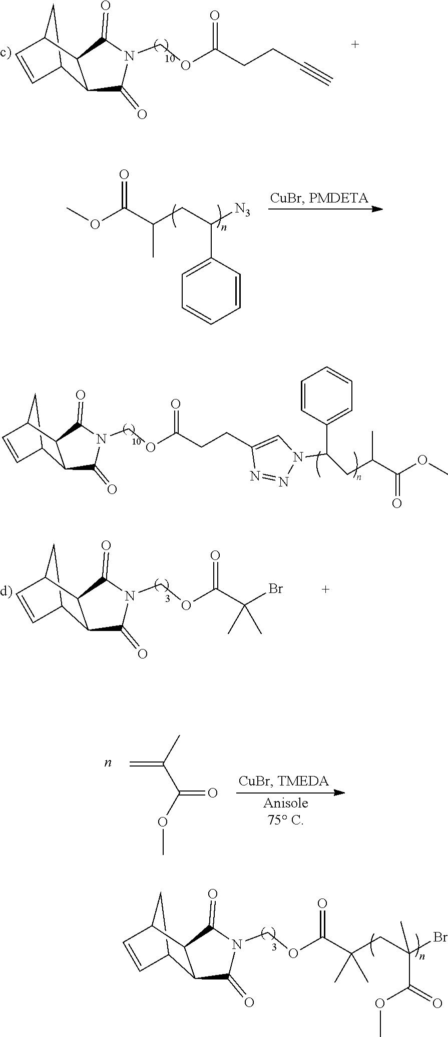

PS is prepared by atom transfer radical polymerization (ATRP) where an azide terminal is formed at the polymer chain end after the polymerization reaction so that the norbornene dicarboximide linker can be “clicked” onto the polymer to create PS (NB-PS) macromonomer (Schemes 4.4a to 4.4c).

PMMA (NB-PMMA) macromonomer is prepared using atom transfer radical polymerization (ATRP). N-(Hydroxypropyl)-cis-5-norbornene-exo-2,3-dicarboximide (NPH) is first reacted with 2-bromoisobutyryl bromide to provide the norbornenyl-functionalized ATRP initiator. NB-PMMA macromonomer is then synthesized by directly growing the polymer from a norbornenyl-functionalized ATRP initiator using CuBr/TMEDA catalytic system (Scheme 4.4d).

Polyamide (PA) macromonomers can be created by ring opening polymerisation of ε-caprolactam on N-(carboxypentyl)-cis-5-norbornene-exo-2,3-dicarboximide (NCP) under reflux conditions using H₂O and H₃PO₃ as catalysts (Scheme 4.5). NCP served as the initiator for ε-caprolactam ROP.

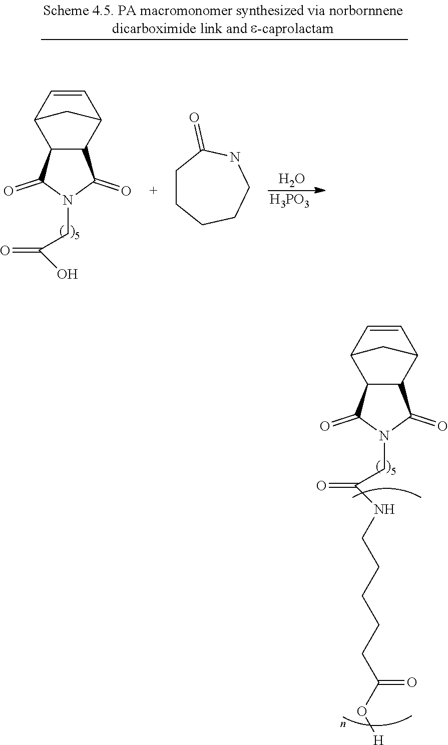

### Example 5: Ring Opening Metathesis Polymerisation Catalysts

With both the bioactive macromonomer and synthetic polymer (PCL, PLA, PLGA, PS, PMMA or PA) macromonomer, the final bioactive copolymer is prepared by ROMP using Grubbs type catalysts 1 or 2 (Scheme 5).

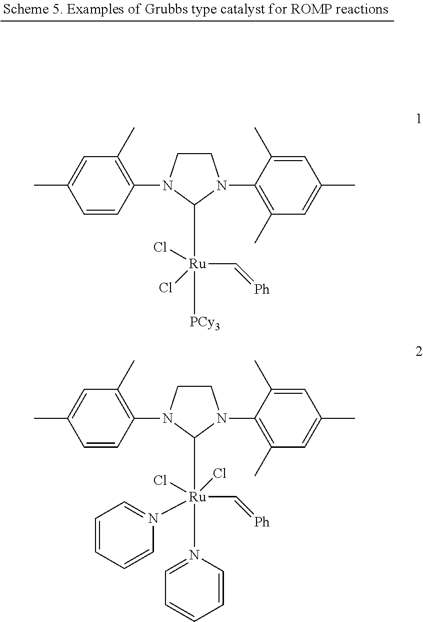

### Example 6: Bioactive Synthetic Copolymers Examples

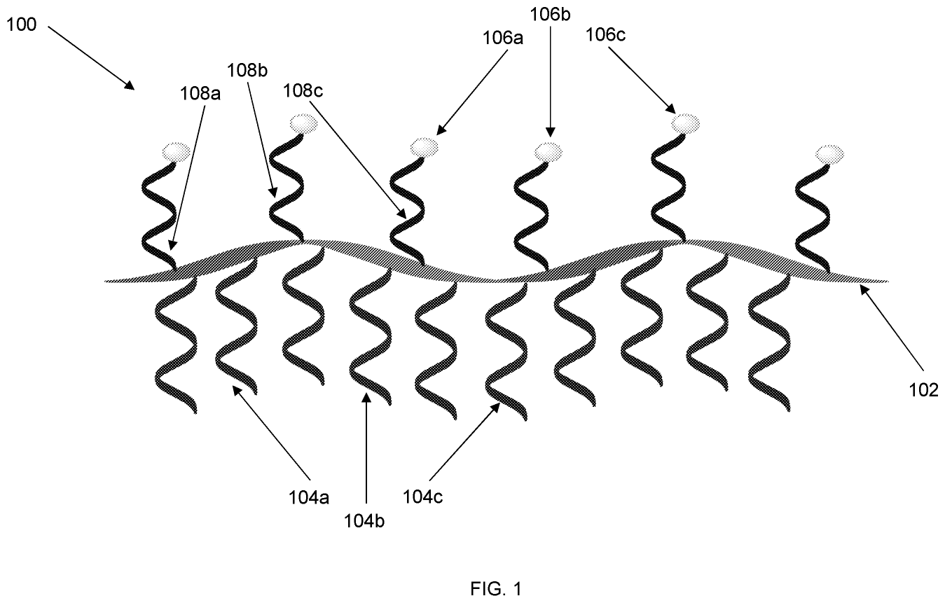

FIG. 1 shows a bioactive synthetic copolymer 100 designed in accordance with various embodiments disclosed herein. The bioactive synthetic copolymer 100 comprises a poly(norbornene dicarboximide) backbone 102, pendant arms of synthetic polymers 104a, 104b and 104c, and pendant arms of bioactive molecules 106a, 106b and 106c tethered on PEG chains 108a, 108b and 108c. As shown in the schematic diagram, the pendant arms are attached to the poly(norbornene dicarboximide) backbone 102. 106a, 106b and 106c may be the same or different types of bioactive moieties.

Examples of synthetic polymers include poly(caprolactone) (PCL), polyesters such as poly(lactic acid) (PLA) and poly(lactic-co-glycolic acid) (PLGA), polystyrene (PS), polyacrylates, poly(meth)acrylates such as poly(methyl methacrylate) (PMMA) and polyamides (PA). Examples of bioactive molecules include biomolecules selected from peptide sequences of 3-20 amino acid residues, formed from 20 natural amino acids, collagen mimic peptides from 3-20 amino acid residues in any sequence such as DGEA, (Gly-Pro-Hyp)₃ and (Pro-Hyp-Gly)₃, carbohydrates such as glycosaminoglycans or drug molecules containing a carboxylic acid terminal such as certain antibiotics.

Depending on the targeted application, the bioactive macromonomer can be matched with different types of synthetic polymers to create materials with different physical properties.

An example would be a bioactive macromonomer comprising RGD. RGD is a peptide sequence that is capable of binding integrins for cell attachment, migration and proliferation. Hence, macromonomer of RGD is created (Scheme 6).

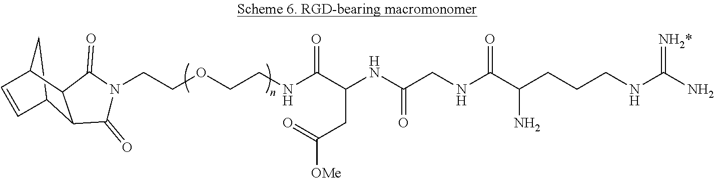

The RGD macromonomer can be paired with a biodegradable macromonomer to create skin scaffolds that would degrade in the human body after the patient's own skin has taken over. This macromonomer can also be copolymerized with heparin sulfate bearing macromonomers and polycaprolactone bearing macromonomers to create triblock copolymers that allow bone tissue regeneration, for use as bioresorbable bone scaffolds. Such a modular approach in building polymers allow the matching of different bioactive macromonomers with synthetic macromonomers to rapidly create mechanically strong therapeutic materials based on patient's needs. The dosage of the therapeutic agent (bioactive macromonomer) can also be tuned to suit a patient's needs by adjusting macromonomer ratios during polymerization.

RGD can be replaced with any peptide sequence via its acid terminal or any carbohydrate or any drug molecule such as amoxicillin or ciprofloxacin that has a carboxylic acid functional group.

For example, glycosaminoglycans (GAGs) such as heparin sulfate (HS) chains of between 5 to 10 disaccharide units may be used as the bioactive moiety. Without being bound by theory, it is believed that HS chains is active toward bone morphogenetic proteins (BMP), in particular, BMP-2, which is able to transdifferentiate myoblasts to osteoblasts. Without being bound by theory, it is believed that DP12, the HS fragment with hexa-disaccharide units, possesses the highest binding affinity for BMP-2. In vitro experiments of BMP-2 complexed with DP12 showed greater osteogenic differentiation in cells whereas in vivo experiments using rat models showed that DP12 enhanced bone tissue regeneration relative to controls of collagen sponge in polycaprolactone (PCL) tubes. Therefore, PCL copolymers with DP12 macromonomers are created which can then be added to base polymer PCL and fabricated into whole bone implants. By chemically linking DP12 to PCL itself before blending the polymers into base polymer PCL, the present disclosure has advantageously shown that it is possible to localize the GAG on the implant to prevent undesirable side effects such as bone tissue regeneration at any other locations of the body except the implant site. Apart from being used as bone scaffolds, the DP14-PCL/PCL blend can also be used to create skin scaffolds since GAGs are also known to enhance keratinocyte regeneration.

Apart from GAGs, peptides such as integrin binders or collagen fragments, which are useful towards skin and bone tissue regeneration may also be used. Extracellular peptides such as RGD are able to function as integrin binders to encourage cell attachment, migration and proliferation.

Apart from RGD peptides, materials may also be created with collagen fragments and mimics. The bone is a mineralized collagenous tissue that remodels itself throughout one's lifecycle to adapt to mechanical stress and maintain the integrity of skeletal tissues. Current bone scaffolds are typically made of collagen sponges, occasionally mineralized with some calcium phosphate ceramics such as tricalcium phosphate or hydroxyapatite. The biocompatibility of collagen and its similarity to bone tissues makes it an ideal scaffold material for bones. Without being bound by theory, it is believed that the use of collagen fragments or collagen mimics (COL) in the PCL scaffolds helps to increase the biocompatibility and biomimetic properties of the overall PCL-based scaffold material. Some possible collagen mimics that may be used include DGEA and collagen fragments bearing varying lengths of glycine, proline and hydroxyproline sequences. Without being bound by theory, it is believed that DGEA supports mesenchymal stem cell adhesion and differentiation to osteoblasts. Furthermore, without being bound by theory, it is believed that collagen also makes excellent skin scaffold materials since the extracellular matrix (ECM) is largely collagenous material. Other ECM peptides studied include laminin-derived peptide A5G81, which has been reported to facilitate wound healing in rats.

Besides tissue-regenerating biomolecules, cell-penetrating peptides such as (IRIK)₂ and (IKKI)₃ may also be used as the bioactive moiety, for incorporation into non-biofouling materials. Biofouling is a serious problem in biomedical devices such as catheters, gut stents and even wound dressings. The ability to target biofilm-forming bacteria such as P. aeruginosa whilst not being toxic to human, makes such peptides attractive candidates for biomedical device materials.

Drug molecules such as antibiotics may also be incorporated into the brush polymers. Theoretically, any drug molecule with a carboxylic acid terminal would allow the creation of these bioactive synthetic polymers. Some of the drug molecules successfully polymerized include amoxicillin and ciprofloxacin. Brush polymers have been created, also for use in antimicrobial devices.

In summary, a general strategy has been developed to create bioactive macromonomers for rapid building of bioactive synthetic polymers by using a modular approach to polymer design and synthesis. Such a modular approach to therapeutic material synthesis allows for therapy customization to suit each patient's needs, hence moving closer to the ideal situation of personal medicine.

In summary, a series of polymers that bear synthetic polymers such as polystyrene, polyacrylate, poly(meth)acrylate, poly(lactide), poly(lactic-co-glycolic acid), poly(ε-caprolactone) and polyamide with pegylated biomolecules as side chains on a poly(norbornene dicarboximide) backbone have been developed, via ROMP technologies. The biocompatibility, bone growth factor and skin cell viability of some of these polymers were also tested to demonstrate the materials' ability to withstand harsh material processing temperatures without loss in bioactivity. The general strategy presented here forms a method to create bioactive synthetic polymers for use as bioadditives in materials for biomedical devices where the bioadditive can be blended with a base polymer of similar type to the polymer side chain on the poly(norbornene dicarboximide) backbone. The synthetic polymer side chain helps make the biomolecule more compatible with the base synthetic polymer, allowing them to be blended together without phase separation. The formation of the brush polymer also allows the biomolecule to have better structural integrity as compared to the native biomolecule itself, which tends to be extremely hygroscopic, resulting in their poor handling and low processability, as a material.

### Experimental Procedures

### General Procedure

Ring opening metathesis polymerization (ROMP) reactions, PS (NB-PS) and PCL (NPH-PCL), macromonomer synthesis, bioactive macromonomer syntheses and catalyst 2 synthesis were carried out in a Vacuum Atmosphere glovebox under nitrogen atmosphere. NBPEG and NB amino alcohol condensation reactions to obtain N-(Hydroxypropyl)-cis-5-norbornene-exo-2,3-dicarboximide (NPH), N-(carboxypentyl)-cis-5-norbornene-exo-2,3-dicarboximide (NCP) and N-(Hydroxydecanyl)-cis-5-norbornene-exo-2,3-dicarboximide (NDH), were carried out in a fumehood under atmospheric conditions. All solvents used in the glovebox are anhydrous and used as purchased. Grubbs second generation catalyst (catalyst 1) was purchased from Sigma Aldrich and peptides were purchased from Biomatik Inc. PEG diamine was purchased from Alfa Aesar (1,000 and 3,400) or Sigma Aldrich (6,000). HOBT, HBTU, ⁱPr₂EtN were purchased from Sigma Aldrich and cis-norbornene-exo-2,3-dicarboxylic anhydride was purchased from Alfa Aesar. DP12 was purchased from Iduron. All purchased reagents were used without further purification.

¹H NMR spectra were recorded on a JEOL 500 MHz NMR spectrometer using MeOD as solvent for all biomolecule-based macromonomers. CDCl₃ is used as solvent for PCL macromonomer. Gel Permeation Chromatography was carried out on a Waters Aquity APC System equipped with Acquity APC XT 45, XT 200 and XT 450 columns, Acquity RI detector. THF was used in sample preparation and a flow rate of 1.0 ml/min at 40° C. was used. Polystyrene was used as calibration standard. TGA/DSC is measured using TA Instruments SDT2960 simultaneous DSC-TGA.

### Synthesis of (H₂IMes)(pyr)₂(Cl)₂RuCHPh (Catalyst 2)

Pyridine (2 mL) was added to catalyst 1 (0.5 g, 0.59 mmol) in a 20 mL vial with a screw cap. The reaction was stirred at room temperature for 15 min during which a colour change from red to green was observed. Hexanes (16 mL) was added to the green solution and a green solid began to precipitate. The green precipitate was vacuum-filtered, washed with hexanes (4×10 mL), and dried under vacuum to afford catalyst 2 as a green powder.

### Synthesis of N-(Hydroxypropyl)-cis-5-norbornene-exo-2,3-dicarboximide (NPH)

A round-bottom flask was charged with cis-5-norbornene-exo-2,3-dicarboxylic anhydride (0.985 g, 6.0 mmol) and 3-amino-1-propanol (0.473 g, 6.3 mmol). To the flask was added 30 mL toluene, followed by triethylamine (84 μL, 0.60 mmol). A Dean-Stark trap was attached to the flask, and the reaction mixture was heated at reflux (135° C.) for 4 h. The reaction mixture was then cooled and concentrated in vacuo to yield a pale yellow oil. This residue was diluted with 30 mL of dichloromethane and washed with 0.2 M HCl (20 mL) and sat. NaCl (20 mL). The organic layer was dried over Na₂SO₄, concentrated in vacuo and dried overnight in a vacuum oven to yield 1.22 g of white solid. ¹H NMR (500 MHz, CDCl₃): δ 6.27 (t, J=2.0 Hz, 2H), 3.64 (t, J=6.4 Hz, 2H), 3.53 (q, J=6.1 Hz, 2H), 3.26 (s, 2H), 2.71 (m, 2H), 2.60 (m, 1H), 1.84-1.70 (m, 2H), 1.55 (m, 1H), 1.24 (d, 1H).

### Synthesis of NPH-PCL Macromonomer by ROP

NPH-PCL macromonomers with different degree of polymerization (DP) were prepared by ROP. As an example, ε-CL (0.5 ml, 0.52 mol) was added to a 20 ml scintillation vial containing NPH initiator (0.05 g, 0.23 mmol), dissolved in toluene (1 ml). Sn(Oct)₂ (0.0037 g, 9.1 μmol) was added to the mixture and the resultant solution was stirred at 110° C. for 90 min and precipitated into methanol. The methanolic solution was then placed in the freezer overnight to result in white precipitate which was filtered and washed with methanol. The residue is then dried under vacuum overnight. GPC analysis (THF): Mₙ=5,613, PDI=1.08, yield 0.4478 g.

A standard solution of Sn(Oct)₂ of concentration 91 μmol/ml, was prepared and used for ROP reactions.

### Synthesis of NPH-PLA Macromonomer by ROP

NPH-PLA macromonomers with different degrees of polymerization (DP) were prepared by ROP. As an example, a flame-dried 25 mL Schlenk tube was charged with NPH initiator (110 mg, 0.50 mmol), D, L-lactide (864 mg, 6.0 mmol), Sn(Oct)₂ (2 mg), and a stir bar. The tube was evacuated and backfilled with nitrogen four times, and was then immersed in an oil bath at 130° C. After 2.5 h, the contents were cooled to room temperature, diluted with dichloromethane, and precipitated into cold methanol twice. The macromonomer was isolated by decanting the supernatant and dried under vacuum overnight. GPC analysis (THF): Mₙ=2,471, PDI=1.20, yield 0.600 g. ¹H NMR (CDCl₃): δ 6.28 (br t, 2H), 5.27-5.08 (m), 4.35 (m, 1H), 4.19-4.02 (m, 2H), 3.62-3.44 (m, 2H), 3.27 (s, 2H), 2.69 (m, 2H), 1.97-1.47 (m), 1.19 (d, 1H).

### Synthesis of N-(Hydroxydecanyl)-cis-5-norbornene-exo-2,3-dicarboximide (NDH)

A round-bottom flask was charged with cis-5-norbornene-exo-2,3-dicarboxylic anhydride (0.95 g, 5.8 mmol) and 10-amino-1-decanol (1.0 g, 5.8 mmol). To the flask was added 20 mL of toluene, followed by triethylamine (80 μL, 0.58 mmol). A homogeneous solution was obtained upon heating. A Dean-Stark trap was attached to the flask, and the reaction mixture was heated at reflux (135° C.) for 4 h. The reaction mixture was then cooled and concentrated in vacuo to yield an off-white solid. This residue was dissolved in 20 mL of CH₂Cl₂ and washed with 0.1 N HCl (10 mL) and sat. NaCl (10 mL). The organic layer was dried over MgSO₄ and concentrated in vacuo to yield 1.96 g of colorless, viscous oil. ¹H NMR (500 MHz, CDCl₃): δ 1.20-1.28 (m, 13H), 1.49-1.56 (m, 5H), 2.65 (d, J=1.5 Hz, 2H), 3.26 (t, J=1.5 Hz, 2H), 3.44 (t, J=7.5 Hz, 2H), 3.62 (t, J=6.5 Hz, 2H), 6.27 (t, J=2.0 Hz, 2H).

### Synthesis of N-(Pentynoyldecanyl)-cis-5-norbornene-exo-2,3-dicarboximide

To a round-bottom flask were added N-(hydroxydecanyl)-cis-5-norbornene-exo-2,3-dicarboximide (NDH) (0.80 g, 2.5 mmol), N-(3-dimethylaminopropyl)-N′-ethylcarbodiimide hydrochloride (EDC) (0.58 g, 3.0 mmol), and 4-dimethylaminopyridine (DMAP) (0.10 g, 0.82 mmol), followed by 10 mL of CH₂Cl₂. Pentynoic acid (0.25 g, 2.5 mmol) was added as a solution in 5 mL of CH₂Cl₂ via syringe. The reaction mixture was allowed to stir at room temperature overnight. The reaction mixture was washed with water (2×20 mL) and sat. NaCl (20 mL) and dried over MgSO₄. The solvent was evaporated, and the remaining residual was purified by silica gel chromatography (ethyl acetate/hexanes, 1:9 v/v) to give 0.88 g product as a colorless oil (88% yield). ¹H NMR (500 MHz, CDCl₃): δ 1.21-1.33 (m, 13H), 1.49-1.54 (m, 3H), 1.62 (t, J=7.5 Hz, 2H), 1.97 (t, J=2.5 Hz, 1H), 2.48-2.57 (m, 4H), 2.67 (d, J=1.5 Hz, 2H), 3.27 (t, J=1.5 Hz, 2H), 3.45 (t, J=7.5 Hz, 2H), 4.09 (t, J=7 Hz, 2H), 6.28 (t, J=2.0 Hz, 2H).

### Synthesis of NB-PS Macromonomer by ATRP-Click

NB-PS macromonomers with different degree of polymerization (DP) were prepared using ATRP, click-reactions. As an example, CuBr (0.1435 g, 1 mmol) was weighed into a 20 ml scintillation vial in the glovebox. Styrene (pre-filtered through basic Al₂O₃, 11.5 ml, 100 mmol) was added followed by methyl-2-bromopropionate (112 μl, 1 mmol) and PMDETA (209 μl, 1 mmol). The mixture was heated at 80° C. for 1 h and added dropwise to stirring MeOH (400 ml) to give a white precipitate (ppt) in deep blue solution. The ppt was filtered to obtain a blueish white solid that is redissolved in minimal CH₂Cl₂, reprecipitated in MeOH and filtered. This redissolving, precipitation and filtration process is repeated until a pure white solid of PS-Br is obtained. The solid is then dried in a vacuum oven overnight. GPC analysis (THF): Mₙ=2,523, PDI=1.18.

PS-Br (0.5 mmol) and NaN₃ (2.5 mmol) were added to a 20 ml scintillation vial in the glovebox, followed by DMF (10 ml) and the mixture was stirred for 48 h to result in a colourless solution with white precipitate of NaBr. The mixture was added to a beaker of stirring MeOH in the fumehood. The white precipitate was filtered, washed with MeOH and dried in a vacuum oven to give PS-N₃ prepolymer.

In a 20 ml scintillation vial was added PS-N₃ prepolymer (0.1 mmol) and N-(Pentynoyldecanyl)-cis-5-norbornene-exo-2,3-dicarboximide (0.15 mmol) and CuBr (0.01 mmol). THF (2 ml) and PMDETA (0.01 mmol) were added and the mixture stirred at 50° C. overnight. MeOH was added to the cooled reaction mixture to yield a white ppt which was filtered and washed with MeOH, followed by drying in a vacuum oven, to yield NB-PS macromonomer. ¹H NMR (CDCl₃): 7.10-6.46 (m), 6.28 (s, 2H), 5.04-4.94 (m, 1H), 4.13-4.0 (m, 2H), 3.51-3.40 (m, 5H), 3.27 (s, 2H), 2.91-2.86 (m, 2H), 2.67-2.56 (m, 2H), 0.92 (br s, 3H).

### Synthesis of Norbornenyl-Functionalized ATRP Initiator

A round-bottom flask was charged with N-(Hydroxypropyl)-cis-5-norbornene-exo-2,3-dicarboximide (NPH) (0.66 g, 3.0 mmol). To the flask was added dichloromethane (12 mL), followed by triethylamine (0.63 mL, 4.5 mmol). The reaction flask was submerged in an ice-water bath and 2-bromoisobutyryl bromide (0.55 mL, 4.5 mmol) was added dropwise to the reaction mixture. When the addition was completed, the reaction mixture was allowed to stir at room temperature overnight. The reaction mixture was washed with 0.1 M HCl (15 mL), saturated NaHCO₃ solution (15 mL) and sat. NaCl (2×15 mL). The organic layer was dried over Na₂SO₄ and concentrated in vacuo. The residue was purified by silica gel chromatography (dichloromethane) to yield the product as a pale yellow solid (0.80 g, 72%). ¹H NMR (500 MHz, CDCl₃): δ 6.28 (t, J=1.8 Hz, 2H), 4.17 (t, J=6.5 Hz, 2H), 3.61 (t, J=7.1 Hz, 2H), 3.28 (s, 2H), 2.69 (d, J=1.8 Hz, 2H), 1.99-1.96 (m, 8H), 1.52 (m, 1H), 1.21 (d, J=9.9 Hz, 1H).

### Synthesis of NB-PMMA Macromonomer by ATRP

NB-PMMA macromonomers with different degrees of polymerization (DP) were prepared using ATRP. As an example, a 25 mL Schlenk tube was charged with norbornenyl-functionalized ATRP initiator (53 mg, 0.143 mmol), MMA (1.06 mL, 10.0 mmol), anisole (1.0 mL) and TMEDA (0.011 mL, 0.072 mmol). The solution was degassed by three freeze-pump-thaw cycles. During the final cycle, the Schlenk tube was filled with nitrogen, and CuBr (10.3 mg, 0.072 mmol) was quickly added to the frozen reaction mixture. The Schlenk tube was sealed, evacuated, and backfilled with nitrogen three times. The Schlenk tube was thawed to room temperature and the polymerization was conducted in a 70° C. oil bath for 3 h. The mixture was filtered through neutral alumina, precipitated into MeOH and filtered. The solid is then dried in a vacuum oven overnight. GPC analysis (THF): Mₙ=5,158, PDI=1.13. ¹H NMR (CDCl₃): δ 6.30 (s, 2H), 4.17 (m, 2H), 3.76 (m), 3.65-3.59 (m), 3.28 (s, 2H), 2.72 (s, 2H), 2.00-1.69 (m), 1.07-0.75 (m).

### Synthesis of N-(Carboxypentyl)-cis-5-norbornene-exo-2,3-dicarboximide (NCP)

cis-5-norbornene-exo-2,3-dicarboxylic anhydride (4.0 g, 24.3 mmol) and 6-aminohexanoic acid (3.3 g, 25.3 mmol) were weighed into a round-bottom flask. To the solid mixture was added toluene (50 mL) and Et₃N (410 μL, 2.92 mmol). The flask was fitted with a Dean-Stark trap and heated to reflux for 4h. The mixture was then allowed to cool to room temperature and diluted with CH₂Cl₂ (50 mL) and washed with 1 M aqueous HCl (2×20 mL). The organic layer was washed with saturated aqueous NaCl (20 mL), dried with Na₂SO₄, filtered, and concentrated under reduced pressure to provide NCP as a pale yellow solid. ¹H NMR (500 MHz, CD₃OD, 25° C.) δ 6.26 (t, 2H, J=2.0 Hz), 3.44 (m, 2H), 3.25 (m, 2H), 2.66 (d, 2H, J=1.0 Hz), 2.32 (t, 2H, J=7.2 Hz), 1.63 (m, 2H), 1.55 (m, 2H), 1.46-1.51 (m, 1H), 1.33 (m, 2H), 1.19 (d, 1H).

### Synthesis of NCP-PA 6 Macromonomer by ROP

NCP-PA 6 macromonomers with different degree of polymerization (DP) were prepared by ROP. As an example, ε-caprolactam (2.56 g, 12 mmol) was weighed into a 50 ml round bottom flask (rbf) containing NCP initiator (0.2 g, 0.6 mmol) with nitrogen inlet. Deionized H₂O (5 ml) with H₃PO₃ (0.081 g) were added to the mixture and the resultant mixture was heated at 170° C. for 30 min and maintained at 240° C. for 4 hrs. H₂O was removed by distillation and the reaction was heated at 240° C. under vacuum for another 2 hrs. Beige solid was precipitated from MeOH and washed repeatedly by it. NCP-PA 6 was obtained upon drying in a vacuum oven overnight. ¹H NMR [500 MHz, DCO₂D/CD₂Cl₂ (1:4),]: δ 6.42 (br, PA 6), 6.28 (s, 2H, NCP), 3.42 (s, 6H, NCP), 3.14-3.12 (m, PA 6), 2.67 (s, 2H, NCP), 2.14-2.12 (m, PA 6), 1.56-1.53 (m, PA 6), 1.46-1.44 (m, PA 6), 1.29-1.25 (m, PA 6).

### Synthesis of NBPEG macromonomer body (for H₂N-PEG-NH₂ 1000, 3,400 and 6000)

PEG diamine (1 g) and cis-norbornene-exo-2,3-dicarboxylic anhydride (1 eq.) were added to a 100 ml rbf, followed by toluene (50 ml). Triethylamine (1 eq.) was added and the mixture stirred under reflux overnight, with a dean stark trap attached for water removal. The resulting solution was evaporated to dryness and dichloromethane (40 ml) was added, followed by 0.1 M HCl (40 ml). The organic layer was extracted and washed with 0.1 M NaOH (50 ml). 0.1 M NaOH (50 ml) was added to the aqueous fraction from the acid wash followed by CH₂Cl₂ (30 ml). The organic layer was extracted and combined, washed with sat. NaCl before drying over Na₂SO₄. The material was evaporated to dryness to give a pale orange oil, NBPEG for PEG diamine 1,000 and beige solid for PEG diamine 3,400 and 6,000. ¹H NMR (MeOD): δ=6.36 (t, 2H, NB), 3.67 (s, PEG), 3.21 (s, 2H, NB), 2.74 (s, 2H, NB), 1.92 (s, 2H).

### Synthesis of NBPEG₁₀₀₀RGD as Representative Prep for PEG 1000, 3,400 and 6,000

RGD (with 1 carboxylic acid end on aspartic acid protected with OMe) (0.0937 g, 0.26 mmol), was dissolved in MeOH (2.5 ml) in a 4 ml vial, in the glovebox. ⁱPr₂EtN (91 μL, 0.52 mmol) was added and the mixture stirred (A). HOBT (0.0353 g, 0.26 mmol) and HBTU (0.0992 g, 0.26 mmol) were dissolved in MeOH (12.5 ml) in a 20 ml vial at 40° C., followed by addition of the RGD solution from (A), to give solution (B). Solution B is then added to NBPEG₁₀₀₀ (0.25 g, 0.218 mmol) in a 40 ml vial and stirred at rt overnight. The resultant mixture is then evaporated to dryness and the oil is added to diethylether (50 ml). The diethylether solution is chilled in a freezer for 48 h and decanted. MeOH (5 ml) is added to the residue to give an orange solution with white ppt. The mixture is passed through a syringe filter and the clear filtrate is evaporated to dryness to give an orange oil of RGDPEGNB at 95% yield. ¹H NMR (MeOD): δ=7.74 (dd), 7.35-7.42 (m), 6.32 (t), 4.39 (s), 4.20 (s), 3.63 (br s), 3.60 (d), 3.17 (t), 2.70 (d). MALDI-MS: 661.3 ([M−NB]+2H⁺).

### Synthesis of NBPEG₁₀₀₀DGEA as Representative Prep for PEG 1000, 3,400 and 6,000

DGEA (with carboxylic acid on E and A protected with OMe) (0.109 g, 0.26 mmol) was dissolved in MeOH (2.5 ml) in glovebox. ⁱPr₂EtN (91 μl, 0.52 mmol) was added and mixture stirred as solution A. HOBT (0.0353 g, 0.26 mmol) and HBTU (0.0992 g, 0.26 mmol) were dissolved in MeOH (12.5 ml) at 40° C., followed by addition of the solution A to give suspension B. Suspension B was then added to NBPEGNH₂ (0.25 g, 0.218 mmol) and stirred at r.t. for 24 h. The resulting pale yellow mixture was then concentrated by solvent evaporation to give yellow oily mixture. The mixture was dispersed into Et₂O and the solution placed in a freezer for 48 h. The Et₂O layer was withdrawn and MeOH was added to the residue to give a yellow suspension. Yellow oily product NB-PEG-DGEA (0.28 g, yield 73%) was obtained upon filtration and solvent evaporation.

¹H NMR (500 MHz, CD₃OD, 25° C.): δ 7.80 (d, 1H), 7.71 (d, 1H), 7.44-7.38 (m, 2H), 6.33 (s, 2H), 4.40 (s, 2H), 4.22 (s, 1H), 3.95 (s, 1H), 3.68 (m, 6H), 3.64 (m, 84H), 3.57 (m, 4H), 3.18 (s, 2H), 2.82 (s, 2H), 2.72 (s, 2H), 2.47 (m, 2H), 2.14 (m, 1H), 1.96 (m, 1H), 1.48-1.41 (dd, 2H).

### Synthesis of NBPEG₁₀₀₀(GPHyp)₃ as Representative Prep for Collagen Fragments of Glycine, Proline and Hydroxyproline in Varying Sequence and Chain Length Up to n=6, PEG 1000, 3,400 and 6,000

(GPHyp)₃ (0.213 g, 0.26 mmol) was dissolved in MeOH (2.5 ml) in glovebox. ⁱPr₂EtN (91 μl, 0.52 mmol) was added and mixture stirred (solution A). HOBT (0.0353 g, 0.26 mmol) and HBTU (0.0992 g, 0.26 mmol) were dissolved in MeOH (12.5 ml) at 40° C., followed by addition of the solution A to give suspension B. Suspension B was then added to NBPEGNH₂ (0.25 g, 0.218 mmol) and stirred at r.t. for 24 h. The resulting pale yellow mixture was then concentrated by solvent evaporation to give beige mixture. The mixture was dispersed into Et₂O and freeze for 48 h. The Et₂O layer was withdrawn and MeOH was added to the residue to give a beige suspension. Beige oily product NB-PEG-(GPHyp)₃ (0.22 g, yield 50%) was obtained upon filtration and solvent evaporation.

¹H NMR (500 MHz, CD₃OD, 25° C.): δ 6.33 (s, 2H), 4.73-4.44 (br, 4H), 3.65 (m, 84H), 3.57 (m, 4H), 3.18 (s, 2H), 2.72 (s, 2H), 2.39-1.80 (br, 8H), 1.44-1.37 (dd, 2H).

### Synthesis of NBPEG₃₄₀₀DP12

DP12 (0.0211 g, 8.5 μmol) was dissolved in MeOH (1.5 ml) in an 8 ml scintillation vial. ⁱPr₂EtN (3 μl, 17 μmol) was added and mixture stirred (solution A). HOBT (0.0032 g, 8.5 μmol) and HBTU (0.0012 g, 8.5 μmol) were dissolved in MeOH (2.5 ml) at 40° C., followed by addition of the solution A to give suspension B. Suspension B was then added to NBPEG₃₄₀₀NH₂ (0.025 g, 7.05 μmol) and stirred at r.t. for 24 hr. The resulting pale yellow mixture was then concentrated with solvent evaporation to give beige mixture. The mixture was dispersed into Et₂O and the mixture placed in freezer for 84 h. The Et₂O was decanted and MeOH was added to the residue to give a pale yellow solution which resulted in an orange oil on filtration of solution followed by solvent evaporation.

¹H NMR (500 MHz, CD₃OD, 25° C.): δ7.81 (dd, 4H), 7.44-7.52 (m), 6.36 (t, 2H), 4.28 (br, 4H), 3.67 (s, 304H), 3.21 (s, 2H), 2.77 (s, 2H), 1.48-1.32 (m, 10H).

### Typical Procedure for ROMP of NPH-PCL Macromonomer and NBPEG₃₄₀₀RGD Macromonomer as Representative Prep for PCL-Peptide Type Copolymers

NBPEG₃₄₀₀RGD macromonomer (0.2 eq.) is weighed into a 4 ml scintillation vial followed by addition of NPH-PCL (0.05 g). THF (0.021 M wrt. NPH-PCL) is added and the mixture stirred at r.t. till a clear solution is obtained. A solution of catalyst 1 or 2 in THF (1.25 mol %, 0.05 M) is added to the solution and the reaction is stirred for 2 h at 30° C. Ethyl vinyl ether is added to the reaction mixture followed by MeOH (3 ml) and the mixture placed in the freezer for 1 h to give a white ppt. The mixture was centrifuged and mother liquor was decanted. The residue was resuspended in methanol, centrifuge followed by decanting mother liquor again, to wash the residue. The washing with MeOH was carried out 3 times before the final residue was dried overnight in a vacuum oven.

For PCL-RGD copolymer, GPC analysis (THF): Mₙ=140,000, PDI=1.21.

### Typical Procedure for ROMP of NPH-PLA Macromonomer and NBPEG₁₀₀₀RGD Macromonomer as Representative Prep for PLA-Peptide Type Copolymers

NBPEG₁₀₀₀ RGD macromonomer (0.1 eq.) is weighed into a 4 ml scintillation vial followed by addition of NPH-PLA (0.05 g). THF (0.05 M wrt. NPH-PCL) is added and the mixture stirred at r.t. till a clear solution is obtained. A solution of catalyst 2 in THF (1.25 mol %) is added to the solution and the reaction is stirred for 1 h. Ethyl vinyl ether is added to the reaction mixture followed by MeOH (3 ml) and the mixture placed in the freezer for 1 h to give a sticky solid. The mother liquor was decanted and the residue washed repeatedly with MeOH followed by drying in vacuum oven. For PLA-RGD copolymer, GPC analyses (THF): Mₙ=76,681, PDI=1.44.

### Typical Procedure for ROMP of NB-PS Macromonomer and NBPEG₁₀₀₀RGD Macromonomer as Representative Prep for PS-Peptide Type Copolymers

NBPEG₁₀₀₀ RGD (0.1 eq.) was weighed into a 4 ml glass vial followed by addition of NB-PS (0.05 g). THF (0.6 ml) is added and the mixture stirred at 25° C. till a clear solution is obtained. A solution of catalyst 1 or 2 in THF (1.25 mol %, 0.05 M) is added to the solution and the reaction is stirred for 1 h. Ethyl vinyl ether is added to the reaction mixture followed by MeOH (3 ml) and the mixture placed in the freezer for 1 h to give a white precipitate. The mixture was filtered and the residue washed repeatedly with MeOH followed by drying in vacuum oven.

For PS-RGD copolymer, GPC analysis (THF): Mₙ=33,484, PDI=1.41.

### Typical Procedure for ROMP of NB-PMMA Macromonomer and NBPEG₁₀₀₀RGD Macromonomer as Representative Prep for PMMA-Peptide Type Copolymers

NBPEG₁₀₀₀ RGD macromonomer (0.1 eq.) is weighed into a 4 ml scintillation vial followed by addition of NB-PMMA (0.05 g). THF (0.05 M wrt. NB-PMMA) is added and the mixture stirred at r.t. till a clear solution is obtained. A solution of catalyst 2 in THF (1.25 mol %) is added to the solution and the reaction is stirred for 1 h. Ethyl vinyl ether is added to the reaction mixture followed by MeOH (3 ml) and the mixture placed in the freezer for 1 h to give a white precipitate. The residue was resuspended in methanol, centrifuged followed by decanting mother liquor again, to wash the residue. The washing with MeOH was carried out 3 times before the final residue was dried overnight in a vacuum oven.

For PMMA-RGD copolymer, GPC analysis (THF): Mₙ=68,512, PDI=1.72.

### Typical Procedure for ROMP of NCP-PA6 Macromonomer and NBPEG₃₄₀₀DGEA Macromonomer as Representative Prep for PA-Peptide Type Copolymers

NBPEG₃₄₀₀DGEA macromonomer (0.2 eq.) is weighed into a 4 ml glass vial followed by addition of NCP-PA6 (0.12 g). CH₃CO₂H (0.021 M wrt. NCP-PA6) is added and the mixture stirred at 80° C. till a clear solution is obtained. Catalyst 2 (1.25 mol %, 0.05 M in CH₂Cl₂) is added to the solution and the reaction is stirred for 24 h at 80° C. Ethyl vinyl ether is added to the reaction followed by MeOH. The mixture was placed in the freezer for 1d to give beige ppt. The suspension was centrifuged and mother liquor was decanted. The residue was washed repeatedly with MeOH followed by drying in vacuum oven to give a beige solid product. ¹H NMR [500 MHz, DCO₂D/CD₂Cl₂ (1:4)]: δ 6.42 (br, PA 6), 3.60 (s, PEG), 3.19-3.13 (m, PA 6), 2.15-2.12 (m, PA 6), 1.62-1.56 (m, PA 6), 1.48-1.42 (m, PA 6), 1.29-1.23 (m, PA 6).

### Example 7: Bioactive Synthetic Copolymers Examples—Poly(ε-Caprolactone)-Biomolecule Copolymers as Bioadditives for Human Skin and Bone Tissue Regeneration

A series of poly(s-caprolactone) (PCL) copolymers with various pegylated biomolecules such as collagen mimics (COL), integrin binding peptides and glycosaminoglycans (GAGs), have been synthesized and characterized. Such copolymers can be used to create tissue regenerating scaffolds in human for either bone or skin regeneration.

PCL is the synthetic polymer of choice in this example due to its ability to biodegrade in human body without causing local acidity like poly(lactic acid) (PLA) and the material is biocompatible. The incorporation of biomolecules such as heparin oligosaccharide DP12 into PCL would be desired for bone scaffold materials that enable bone tissue regeneration while the material itself biodegrades in the body eventually.

However, the problem with DP12 is that it is extremely hygroscopic and coating it on PCL tubes before implantation is not ideal as there is no means of controlling the homogeneity of the coating prior to use. Furthermore, as the GAG is highly water soluble, it has a high tendency to leach into the body upon implantation and not remain on the implant itself, for BMP binding where bone tissue regeneration is required. Hence, in this example, PCL copolymers with DP12 macromonomers are created which can then be added to base polymer PCL and fabricated into whole bone implants. By chemically linking DP12 to PCL itself before blending the polymers into base polymer PCL, the present disclosure has advantageously shown that it is possible to localize the GAG on the implant to prevent undesirable side effects such as bone tissue regeneration at any other locations of the body except the implant site.

Apart from GAGs, peptides such as integrin binders or collagen fragments, are also useful toward bone tissue regeneration. In fact, these biomimetic molecules (GAGs, integrin binders, collagen fragments) are not only useful for bone tissue regeneration but also skin tissue regeneration. Hence, the polymers synthesized in accordance with various embodiments disclosed herein not only serve as bone scaffolds, they can also be employed in skin scaffolds to allow for skin tissue regeneration in patients with large area wounds such as burns patients.

Extracellular peptides such as RGD are able to function as integrin binders to encourage cell attachment, migration and proliferation. RGD sequence is mostly found in native collagen but is often inaccessible for integrin binding until the collagen is denatured. Hence, it would be useful to isolate RGD sequence from collagen and apply it to the tissue regeneration products directly. RGD can be advantageously used in skin and bone tissue regeneration products as it is able to induce cell growth and angiogenesis through its integrin binding ability. However, as with many biomolecules, it is extremely hygroscopic. In fact, it is more hygroscopic than DP12 where exposure to humid air for 5-10 min turns it from a crystalline solid to liquid immediately. Without anchoring RGD to a synthetic polymer to increase its ease of handling, it is extremely difficult to apply the peptide to the site of repair, especially in a bone defect.

The bone is a mineralized collagenous tissue that remodels itself throughout one's lifecycle to adapt to mechanical stress and maintain the integrity of skeletal tissues. Current bone scaffolds are typically made of collagen sponges, occasionally mineralized with some calcium phosphate ceramics such as tricalcium phosphate or hydroxyapatite. The biocompatibility of collagen and its similarity to bone tissues makes it a desirable scaffold material for bones. In this example, collagen fragments or collagen mimics (COL) were also used in the PCL scaffolds to increase the biocompatibility and biomimetic properties of the overall PCL-based scaffold material. Some collagen mimics such as DGEA and collagen fragments bearing varying lengths of glycine, proline and hydroxyproline sequences have been used as the bioactive moieties (see Scheme 7). Without being bound by theory, it is believed that DGEA supports mesenchymal stem cell adhesion and differentiation to osteoblasts. Apart from being good bone scaffold materials, collagen also make excellent skin scaffold materials since the extracellular matrix (ECM) is largely collagenous material. Hence, the same PCL-COL polymers can also be used as bioadditives to the PCL scaffold matrix for application in skin scaffolds.

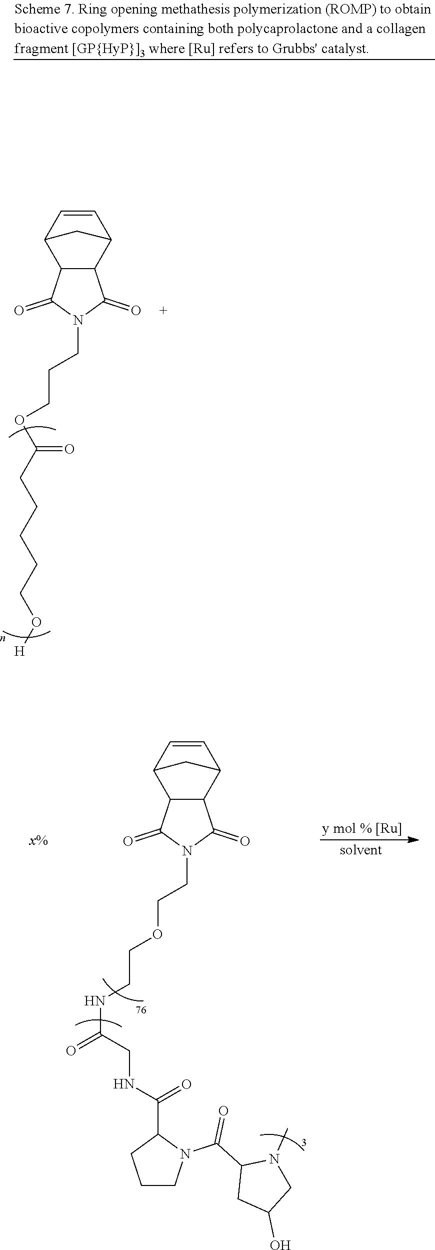

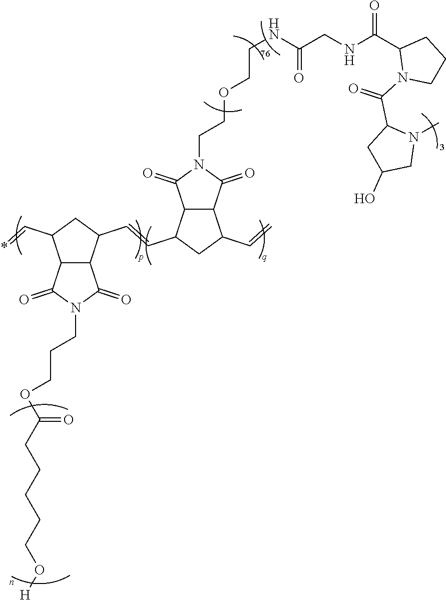

### Thermal Stability

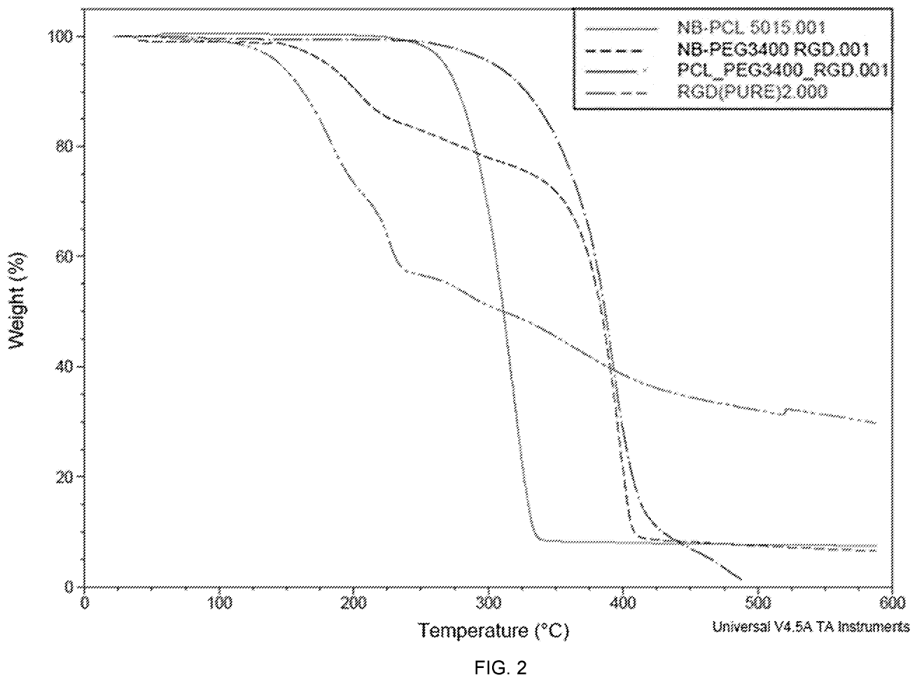

Thermal stability of the biomacromonomer NBPEGRGD and the NBPCL-PEGRGD ROMP polymer has been measured and compared. Pure RGD shows thermal degradation at 181° C. whereas the macromonomer NBPEG₃₄₀₀RGD shows a 2-phase mass loss at 220° C. (RGD loss) and 398° C. (PEG loss). However, on copolymerization with NBPCL macromonomer, the overall bioactive synthetic polymer only shows 1 significant mass loss at 393° C., indicating overall improvement in stability of RGD moiety on the synthetic polymer. In fact, thermal stability of NBPCL macromonomer is also improved with the inclusion of NBPEG₃₄₀₀RGD macromonomer in the overall bioactive synthetic polymer product (FIG. 2).

### Biocompatibility

Copolymers were created using PCL as the synthetic polymer and a range of peptides of different properties as the bioactive macromonomer, namely collagen fragment (GPHyp)₃: GPHP; collagen mimic: DGEA and integrin binding peptides: SRGDS and RGD. The copolymers were subsequently blended with medical grade PCL and 3D-printed into sheets, before tested for cell viability and biocompatibility against commercially available wound dressing namely Allevyn, which is most commonly used in hospitals.

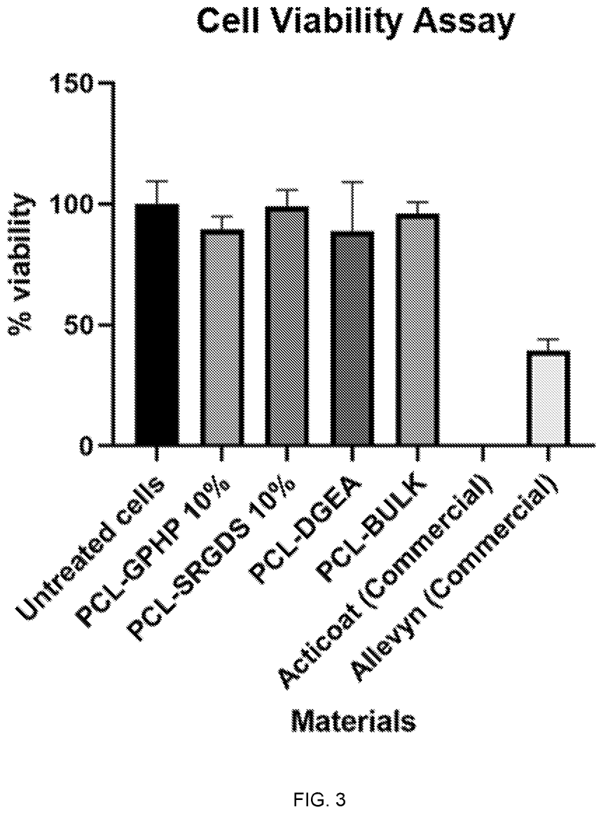

As shown in FIG. 3, all materials designed in accordance with various embodiments disclosed herein showed better cell viability than commercial dressings after a test duration of 72 hours with human skin fibroblasts (Hs27).

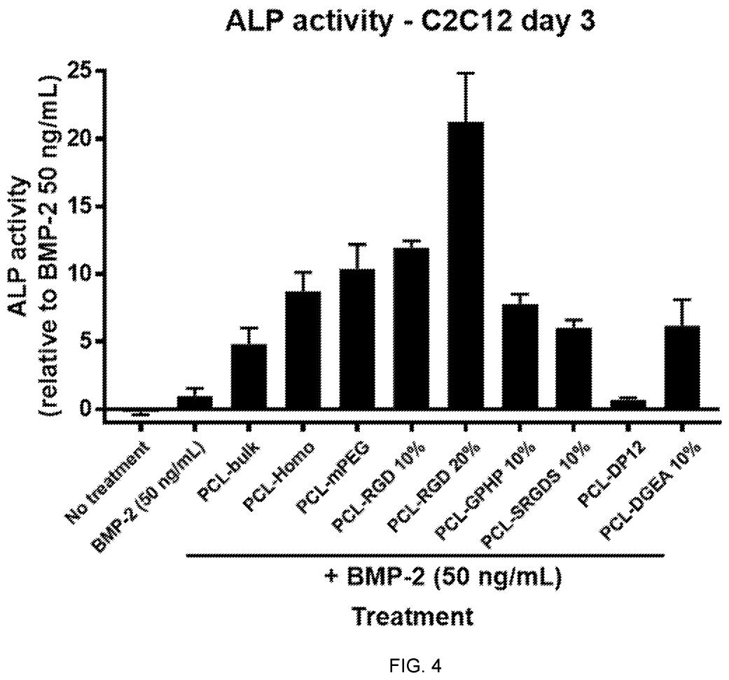

Alkaline phosphatase (ALP) assays to check for osteoblast activity was conducted on the materials to determine the materials' compatibility with BMP-2, a bone growth factor necessary for bone tissue growth relative to pure PCL, a commonly used material for bone scaffolds. Alkaline phosphatase (ALP) is the most widely recognized biochemical marker for osteoblast activity. The osteoinductivity of BMP-2 can be measured in vitro using a pluripotent myoblast C₂Cl₂ cell line. PCL-RGD showed excellent ALP activity compared to PCL. At 20% blending in pure PCL, PCL-RGD showed 4 times higher activity after 72 h of incubation. PCL-(GPHyp) also showed improved activity over PCL (FIG. 4). The ALP assays show the ability of PCL-peptide materials such as RGD and GPHyp, to promote osteogenic activity of cells compared to control of BMP-2 and pure PCL.

In summary, a series of polymers that bear bioresorbable PCL side chains and bioactive molecules such as heparin sulfate DP12, collagen mimics or fragments and integrin binders such as RGD have been developed. These copolymers enhance both bone and skin tissue regeneration and would serve as useful bioactive ingredients for tissue regenerating scaffold materials. A general strategy for creating a scaffold material would be through blending of such bioactive ingredients with a base material of similar nature, that is, medical grade PCL itself.

### Experimental Procedures

### General Procedure

Ring opening metathesis polymerization (ROMP) reactions, PCL macromonomer (NPH-PCL) synthesis, bioactive macromonomer syntheses were carried out in a Vacuum Atmosphere glovebox under nitrogen atmosphere. NBPEG and NPH synthesis was carried out in a fumehood under atmospheric conditions, following procedures provided in Example 6. All solvents used in the glovebox are anhydrous and used as purchased. Grubbs second generation catalyst was purchased from Sigma Aldrich and peptides were purchased from Biomatik Inc. PEG diamine was purchased from Alfa Aesar (1,000 and 3,400) or Sigma Aldrich (6,000). HOBT, HBTU, ⁱPr₂EtN were purchased from Sigma Aldrich and cis-norbornene-exo-2,3-dicarboxylic anhydride was purchased from Alfa Aesar. Heparin oligosaccharide DP12 was purchased from Iduron. All purchased reagents were used without further purification.

¹H NMR spectra were recorded on a JEOL 500 MHz NMR spectrometer using MeOD as solvent for all biomolecule-based macromonomers. CDCl₃ is used as solvent for PCL macromonomer. Gel Permeation Chromatography was carried out on a Waters Aquity APC System equipped with Acquity APC XT 45, XT 200 and XT 450 columns, Acquity RI detector. THF was used in sample preparation and a flow rate of 1.0 ml/min at 40° C. was used.

Synthesis of NBPEG and NBPEGRGD are described in Example 6.

For BMP-2 binding study, scaffolds were sterilized using 100% ethanol, then rinsed in sterile water before being transferred to a 24-well plate. BMP-2 (50 ng in 100 μL PBS) was added directly to the top of each scaffold and incubated for 20 minutes at room temperature. BMP-2 alone was added directly to empty wells. Cells were seeded at 2×104 cells/cm² in 1 mL of 5% FCS media, directly onto the scaffolds and into the surrounding well. Cells were incubated for 72h (37° C., 5% CO₂) prior to ALP assay.

### Synthesis of NPH-PCL Macromonomer by ROP

NPH-PCL macromonomers with different degree of polymerization (DP) were prepared by ROP. As an example, ε-CL (0.5 ml, 0.52 mol) was added to a 20 ml scintillation vial containing NPH initiator (0.05 g, 0.23 mmol), dissolved in toluene (1 ml). Sn(Oct)₂ (0.0037 g, 9.1 μmol) was added to the mixture and the resultant solution was stirred at 110° C. for 90 min and precipitated into methanol. The methanolic solution was then placed in the freezer overnight to result in white precipitate which was filtered and washed with methanol. The residue is then dried under vacuum overnight. GPC analysis (THF): Mₙ=5,613, PDI=1.08, yield 0.4478 g.

A standard solution of Sn(Oct)₂ of concentration 91 μmol/ml, was prepared and used for ROP reactions.

### Synthesis of NBPEG₁₀₀₀DGEA as Representative Prep for PEG 1000, 3,400 and 6,000

DGEA (with carboxylic acid on E and A protected with OMe) (0.109 g, 0.26 mmol) was dissolved in MeOH (2.5 ml) in glovebox. ⁱPr₂EtN (91 μl, 0.52 mmol) was added and mixture stirred as solution A. HOBT (0.0353 g, 0.26 mmol) and HBTU (0.0992 g, 0.26 mmol) were dissolved in MeOH (12.5 ml) at 40° C., followed by addition of the solution A to give suspension B. Suspension B was then added to NBPEGNH₂ (0.25 g, 0.218 mmol) and stirred at r.t. for 24 h. The resulting pale yellow mixture was then concentrated by solvent evaporation to give a yellow oily mixture. The mixture was dispersed into Et₂O and the solution placed in a freezer for 48 h. The Et₂O layer was withdrawn and MeOH was added to the residue to give a yellow suspension. Yellow oily product NB-PEG-DGEA (0.28 g, yield 73%) was obtained upon filtration and solvent evaporation.

¹H NMR (500 MHz, CD₃OD): δ 7.80 (d, 1H), 7.71 (d, 1H), 7.44-7.38 (m, 2H), 6.33 (s, 2H), 4.40 (s, 2H), 4.22 (s, 1H), 3.95 (s, 1H), 3.68 (m, 6H), 3.64 (m, 84H), 3.57 (m, 4H), 3.18 (s, 2H), 2.82 (s, 2H), 2.72 (s, 2H), 2.47 (m, 2H), 2.14 (m, 1H), 1.96 (m, 1H), 1.48-1.41 (dd, 2H).

### Synthesis of NBPEG₁₀₀₀(GPHyp)₃ as Representative Prep for Collagen Fragments of Glycine, Proline and Hydroxyproline in Varying Sequence and Chain Length Up to n=6, PEG 1000, 3,400 and 6,000

(GPHyp)₃ (0.213 g, 0.26 mmol) was dissolved in MeOH (2.5 ml) in glovebox. ⁱPr₂EtN (91 μl, 0.52 mmol) was added and mixture stirred (solution A). HOBT (0.0353 g, 0.26 mmol) and HBTU (0.0992 g, 0.26 mmol) were dissolved in MeOH (12.5 ml) at 40° C., followed by addition of the solution A to give suspension B. Suspension B was then added to NBPEGNH₂ (0.25 g, 0.218 mmol) and stirred at r.t. for 24 h. The resulting pale yellow mixture was then concentrated by solvent evaporation to give beige mixture. The mixture was dispersed into Et₂O and freeze for 48 h. The Et₂O layer was withdrawn and MeOH was added to the residue to give a beige suspension. Beige oily product NB-PEG-(GPHyp)₃ (0.22 g, yield 50%) was obtained upon filtration and solvent evaporation.

¹H NMR (500 MHz, CD₃OD): δ 6.33 (s, 2H), 4.73-4.44 (br, 4H), 3.65 (m, 84H), 3.57 (m, 4H), 3.18 (s, 2H), 2.72 (s, 2H), 2.39-1.80 (br, 8H), 1.44-1.37 (dd, 2H).

### Synthesis of NBPEG₃₄₀₀DP12

DP12 (0.0302 g, 8.5 μmol) was dissolved in MeOH (1.5 ml) in an 8 ml scintillation vial. ⁱPr₂EtN (3 μl, 17 μmol) was added and mixture stirred (solution A). HOBT (0.0032 g, 8.5 μmol) and HBTU (0.0012 g, 8.5 μmol) were dissolved in MeOH (2.5 ml) at 40° C., followed by addition of the solution A to give suspension B. Suspension B was then added to NBPEG₃₄₀₀NH₂ (0.025 g, 7.05 μmol) and stirred at r.t. for 24 hr. The resulting pale yellow mixture was then concentrated with solvent evaporation to give beige mixture. The mixture was dispersed into Et₂O and the mixture placed in freezer for 84 h. The Et₂O was decanted and MeOH was added to the residue to give a pale yellow solution which resulted in an orange oil on filtration of solution followed by solvent evaporation.

¹H NMR (500 MHz, CD₃OD): δ 7.81 (dd, 4H), 7.44-7.52 (m), 6.36 (t, 2H), 4.28 (br, 4H), 3.67 (s, 304H), 3.21 (s, 2H), 2.77 (s, 2H), 1.48-1.32 (m, 10H).

### Typical Procedure for ROMP of NPH-PCL Macromonomer and NBPEGz₃₄₀₀RGD Macromonomer as Representative Prep for PCL-Peptide Type Copolymers

NBPEG₃₄₀₀RGD macromonomer (0.2 eq.) is weighed into a 4 ml scintillation vial followed by addition of NPH-PCL (0.05 g). THF (0.021 M wrt. NPH-PCL) is added and the mixture stirred at r.t. till a clear solution is obtained. A solution of catalyst 1 or 2 in THF (1.25 mol %, 0.05 M) is added to the solution and the reaction is stirred for 2 h at 30° C. Ethyl vinyl ether is added to the reaction mixture followed by MeOH (3 ml) and the mixture placed in the freezer for 1 h to give a white ppt. The mixture was centrifuged and mother liquor was decanted. The residue was resuspended in methanol, centrifuge followed by decanting mother liquor again, to wash the residue. The washing with MeOH was carried out 3 times before the final residue was dried overnight in a vacuum oven.

For PCL-RGD copolymer, GPC analysis (THF): Mₙ=140,000, PDI=1.21.

### Typical Procedure for ROMP of NPH-PCL Macromonomer as Representative Prep for PCL Homopolymer

NPH-PCL macromonomer (0.63 g) is weighed into a 10 ml scintillation vial followed by addition of THF (0.021 M wrt. NPH-PCL) and the mixture stirred at 27° C. till a clear solution is obtained. A solution of catalyst 1 or 2 in THF (1.25 mol %, 0.05 M) is added to the solution and the reaction is stirred for 2 h at 27° C. Ethyl vinyl ether is added to the reaction mixture followed by MeOH (5 ml) and the mixture placed in the freezer for 1 d to give a white ppt. The mixture was filtered and washed with MeOH repeatedly before the final product was dried overnight in a vacuum oven.

¹H NMR (500 MHz, CDCl₃): δ4.07-4.04 (m, PCL), 2.32-2.29 (m, PCL), 1.65-1.62 (m, PCL), 1.37-1.31 (m, PCL). GPC analysis (THF): Mₙ=225,000, PDI=1.09. TGA: 315.7° C.

### Typical Procedure for ROMP of NPH-PCL Macromonomer and NB-mPEG₅₀₀₀ Macromonomer as Representative Prep for PCL-mPEG Type Copolymers

NB-mPEG₅₀₀₀ macromonomer (0.1 eq.) is weighed into a 10 ml scintillation vial followed by addition of NPH-PCL (0.5 g). THF (0.021 M wrt. NPH-PCL) is added and the mixture stirred at 45° C. till a clear solution is obtained. A solution of catalyst 1 or 2 in THF (1.25 mol %, 0.05 M) is added to the solution and the reaction is stirred for 2 h at 45° C. Ethyl vinyl ether is added to the reaction mixture followed by MeOH (5 ml) and the mixture placed in the freezer for 1 d to give a white ppt. The mixture was filtered and washed with MeOH repeatedly before the final product was dried overnight in a vacuum oven.

¹H NMR (500 MHz, CDCl₃): δ 4.07-4.04 (m, PCL), 3.64 (s, PEG), 2.32-2.28 (m, PCL), 1.65-1.62 (m, PCL), 1.38-1.32 (m, PCL). GPC analysis (THF): M˜=171,000, PDI=1.21. TGA: 316.4° C.

### Example 8: Bioactive Synthetic Copolymers Examples—Polyamide-Peptide Brush Polymers for Use as Bioadditives in Biomedical Devices

A series of polyamide (PA) copolymers with various pegylated biomolecules such as collagen mimics and integrin binding peptides have been synthesized and characterized using ring opening metathesis polymerization. The brush polymers can be blended with polymers similar to that on the pendant arms to create bioactive materials for use in biomedical devices such as catheters, plastic surgery implants, prosthetic parts, cartilage joint implants etc.

This example reports another type of bioactive brush polymer using polyamide (PA) and collagen mimics, for use in biomedical devices that are polyamide-based.

Polyamides (PA) is the synthetic polymer of choice in this example. Polyamides (PA) such as PA 6, PA 12, PA 6,6 are silky thermoplastics that have found significant biomedical applications such as in tubings, surgical guides, prosthetics, sutures and ligament, tendon repair. It is believed that PA has the lowest microbial contamination compared to other materials. PA type polymers can be mixed with a wide variety of additives to achieve many different property variations, allowing the devices to be fabricated using a wide variety of material processing methods such as melt extrusion, 3D-printing and injection molding. However, polyamide chain is polymerized under harsh conditions of high temperature with reduced pressure. This polymer is also insoluble in most solvents, adding to the difficulty of this material preparation.

PA-based materials with collagen fragments and mimics have been created using ring opening metathesis polymerization (ROMP) techniques. The biocompatibility of collagen and its similarity to human tissues makes it an ideal material for biomedical device. However, as with many biomolecules, it is extremely hygroscopic. Without anchoring the collagen fragments or mimics to a synthetic polymer to increase its ease of handling, it is nearly impossible to create an implant or biomedical device for insertion into human body. Although cross-linked collagen is often used in wound care products, they are also very hygroscopic, existing as gels upon absorption of moisture, rendering them too weak for use as implantable devices on their own. Furthermore, full-length human collagen requires complex synthesis and often show poor solubility in buffers. Short collagen-mimic peptide sequences or fragments which include crucial peptide sequences at a fraction of the length has been used to elicit similar biological response to their full-length collagen counterparts. Collagen mimics such as DGEA (Asp-Gly-Glu-Ala) and collagen fragments bearing varying lengths of glycine, proline and hydroxyproline sequences are incorporated into the synthetic polymer. DGEA is capable of promoting cell adhesion, spreading and osteogenic differentiation which will be advantageous for applications in both skin and cartilaginous bone regeneration.

On the other hand, polyamide being an FDA-approved polymer for biomedical device usage, still triggers inflammatory responses in the host body as it is after all, a foreign material. Foreign Body Reaction (FBR) may be induced by polyamide, resulting in inflammation around implantation site. Without being bound by theory, it is believed that the use of collagen fragments or collagen mimics (COL) in the polyamide polymeric material can help to increase the biocompatibility and biomimetic properties of the overall polyamide based implant or device. Some possible collagen mimics used include DGEA and collagen fragments bearing varying lengths of glycine, proline and hydroxyproline sequences, in any order. In fact, collagen fragments make excellent skin and bone regeneration materials since the extracellular matrix (ECM) and bone is largely collagenous material. The bone especially, is mineralized collagen and cartilage joints are mostly collagen fibers, glycosaminoglycans and proteoglycans. Hence, the use of collagen-modified polyamide in joint implants may be particularly useful in helping the joints heal by stimulating collagen regeneration at the implantation site. This exact property also makes it suitable for use in plastic surgery implants where cartilaginous bones are required such as in rhinoplasty implants.

With both the biomacromonomer and synthetic PA macromonomer, the final bioactive polymer is prepared by ROMP using Grubbs type catalysts (Scheme 8).

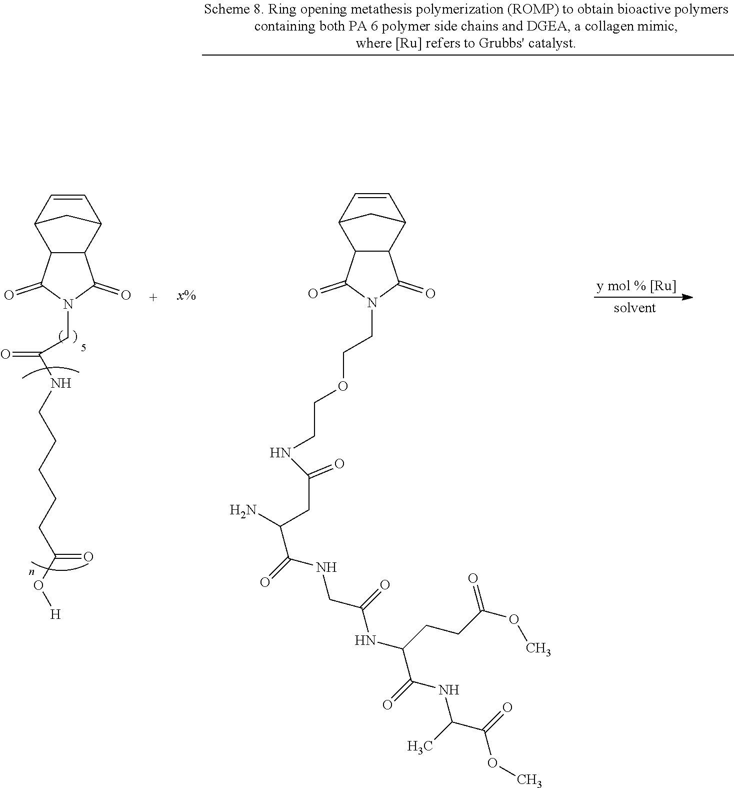

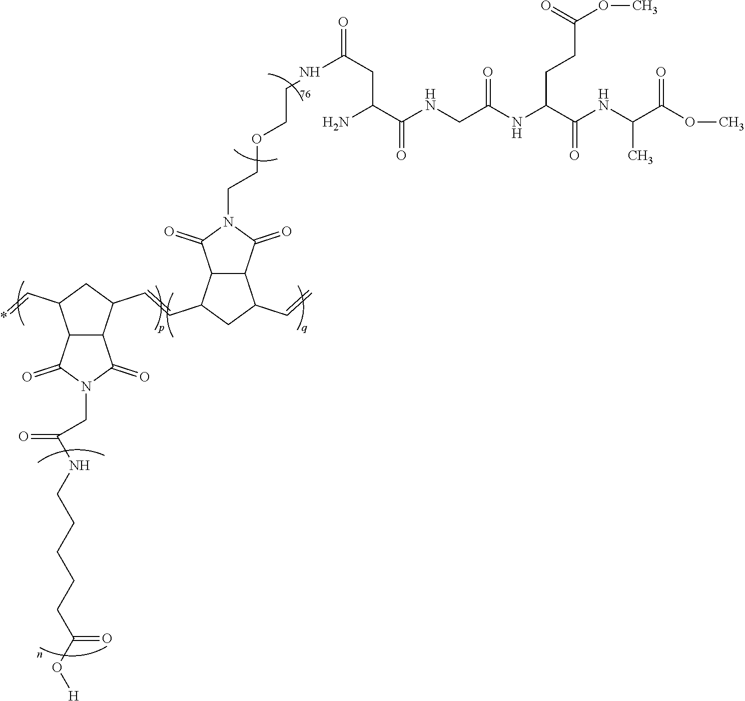

Upon synthesis, metal catalyst removal and characterization, the bioactive polymers are blended with medical grade PA of choice, depending on application, and processed by either fused filament fabrication (fff) or fused deposition modelling (FDM) type 3D printing, melt extrusion, melt blowing or electrospinning into relevant shapes and tested for biocompatibility. TGA-DSC analyses on the synthesized copolymers are typically carried out before material processing to ascertain thermal properties such as T_{g} and degradation temperature of material, prior to processing.

Thermal Stability

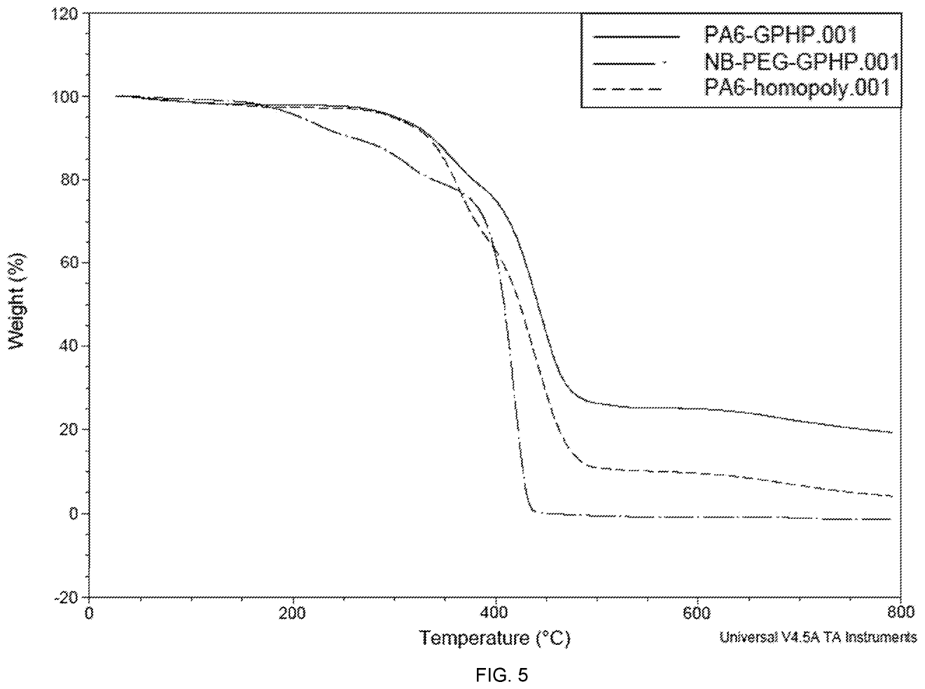

Thermal stability of the biomacromonomer NB-PEG-(GPHyp) and the PA 6 ROMP polymers have been measured and compared (FIG. 5). As can be seen, the copolymer PA6-(GPHyp) only degrades above 450° C. Such high thermal stability allows for a variety of material processing methods such as FFF or FDM type 3D printing, to be conducted on the materials designed in accordance with various embodiments disclosed herein, for implant making.

### Biocompatibility

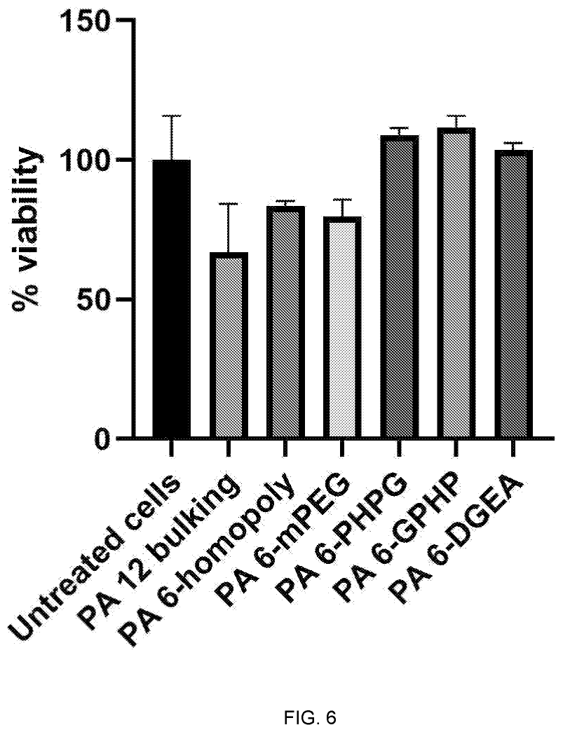

Biocompatibility tests using human fibroblasts Hs27, were carried out on 3 of the PA-collagen materials, PA6-(GPHyp)₃, PA6-(PHypG)₃ and PA6-DGEA, where both (GPHyp)₃ and (PHypG)₃ are collagen fragments and DGEA is a collagen mimic. The bioactive polymers were blended with medical grade PA12, electrospun into sheets of fibers, sterilized with 70% EtOH, dried and incubated for 72 h with human skin fibroblasts (Hs27) before being checked for cell viability using Celltitre-Glo assays. From the cell viability data (FIG. 6), it can be seen that the materials designed in accordance with various embodiments disclosed herein are not only able to maintain better cell viability than controls without collagen, PA6-homopolymer and PA6-mPEG₅₀₀₀, they even showed increased amounts of viable cells, suggesting cell growth even at 72 h. This is in spite of only 2% (GPHyp)₃, in the copolymer, blended with PA 12 in 1:9 ratio (0.2% (GPHyp)₃ in polymer formulation). In fact, the materials significantly improved cell viability over pure medical grade PA12 (Rilsamid®), showing the importance of bioactive polymers in improving biocompatibility of commercial medical grade polymers meant for biomedical device manufacturing. The tests were carried out in triplicates. Different blending ratios and more cell assay tests are ongoing to reaffirm this cell regeneration ability of the materials. Nevertheless, as can be seen, the preliminary results are encouraging.

In summary, a series of brush polymers with polyamide 6 and pegylated biomolecules as side chains, on a poly(norbornene dicarboximide) backbone have been developed, via ROMP technologies. The general strategy presented here forms a method to create bioactive synthetic polymers for use as bioadditives in materials for biomedical devices where the bioadditive can be blended with a base polymer of similar type to the polymer side chain on the poly(norbornene dicarboximide) backbone. In this case, the base polymer is polyamide. The synthetic PA6 side chain helps make the biomolecule more compatible with the base polymer PA12, allowing them to be blended together without phase separation. The formation of the brush polymer also allows the biomolecule to have better structural integrity as compared to the native biomolecule itself, which tends to be extremely hygroscopic, resulting in their poor handling and low processability as a material. Preliminary cell viability tests showed improvements in cell viability from PA6-collagen materials, over PA6 polymers without the biomolecules and significantly greater improvement in cell viability over pure medical grade PA12. Excellent human skin fibroblast cell growth was observed for PA-collagen material, relative to pure PA12.

### Experimental Procedures

### General Procedure

Ring opening metathesis polymerization (ROMP) reactions and bioactive macromonomer syntheses were carried out in a Vacuum Atmosphere glovebox under nitrogen atmosphere. PA 6 macromonomer synthesis was carried out under positive N₂ flow on bench. Reactions to obtain NBPEG and N-(Carboxypentyl)-cis-5-norbornene-exo-2,3-dicarboximide (NCP) were carried out in a fumehood under atmospheric conditions, following procedures provided in Example 6. All solvents used are anhydrous and used as purchased. Grubbs catalyst was purchased from Sigma Aldrich and peptides were purchased from Biomatik Inc. PEG diamine were purchased from Alfa Aesar (1,000 and 3,400) or Sigma Aldrich (6,000). HOBT, HBTU, ⁱPr₂EtN and 2,2,2-trifluoroethanol were purchased from Sigma Aldrich and cis-norbornene-exo-2,3-dicarboxylic anhydride was purchased from Alfa Aesar. Medical grade PA12 (Rilsamid®) for blending was purchased from Arkema. All purchased reagents were used without further purification.

¹H NMR spectra were recorded on a JEOL 500 MHz NMR spectrometer using CD₃OD as solvent for all biomolecule-based macromonomers. DCO₂D/CD₂Cl₂ (1:4) is used as solvent for polyamide peptide macromonomer.

### Synthesis of NBPEG macromonomer body (for H₂N-PEG-NH₂ 1000, 3,400 and 6000)

PEG diamine (1 g) and cis-norbornene-exo-2,3-dicarboxylic anhydride (1 eq.) were added to a 100 ml rbf, followed by toluene (50 ml). Triethylamine (1 eq.) was added and the mixture stirred under reflux overnight, with a dean stark trap attached for water removal. The resulting solution is evaporated to dryness and dichloromethane (40 ml) was added, followed by 0.1 M HCl (40 ml). The organic layer was extracted and washed with 0.1 M NaOH (50 ml). 0.1 M NaOH (50 ml) was added to the aqueous fraction from the acid wash followed by CH₂Cl₂ (30 ml). The organic layer was extracted and combined, washed with sat. NaCl before drying over Na₂SO₄. The material was evaporated to dryness to give a pale orange oil, NBPEG for PEG diamine 1,000 and beige solid for PEG diamine 3,400 and 6,000. ¹H NMR (500 MHz, MeOD): δ=6.36 (t, 2H, NB), 3.67 (s, PEG), 3.21 (s, 2H, NB), 2.74 (s, 2H, NB), 1.92 (s, 2H).

### Synthesis of NBPEG₁₀₀₀DGEA as Representative Prep for PEG 1000-6,000

DGEA (with carboxylic acid on E and A protected with OMe) (0.109 g, 0.26 mmol) was dissolved in MeOH (2.5 ml) in glovebox. ⁱPr₂EtN (91 μl, 0.52 mmol) was added and the mixture stirred as solution A. HOBT (0.0353 g, 0.26 mmol) and HBTU (0.0992 g, 0.26 mmol) were dissolved in MeOH (12.5 ml) at 40° C., followed by addition of solution A to give suspension B. Suspension B was then added to NBPEGNH₂ (0.25 g, 0.218 mmol) and stirred at r.t. for 24 h. The resulting pale yellow mixture was then concentrated by solvent evaporation to give a yellow oily mixture. The mixture was dispersed into Et₂O and the mixture placed in a freezer for 48 h. The Et₂O layer was withdrawn and MeOH was added to the residue to give a yellow suspension. Yellow oily product NB-PEG-DGEA (0.28 g, yield 73%) was obtained upon filtration and solvent evaporation. ¹H NMR (500 MHz, CD₃OD): δ 7.80 (d, 1H), 7.71 (d, 1H), 7.44-7.38 (m, 2H), 6.33 (s, 2H), 4.40 (s, 2H), 4.22 (s, 1H), 3.95 (s, 1H), 3.68 (m, 6H), 3.64 (m, 84H), 3.57 (m, 4H), 3.18 (s, 2H), 2.82 (s, 2H), 2.72 (s, 2H), 2.47 (m, 2H), 2.14 (m, 1H), 1.96 (m, 1H), 1.48-1.41 (dd, 2H).

### Synthesis of NBPEG₁₀₀₀ (GPHyp)ₙ (n=3) as Representative Prep for Collagen Fragments of Glycine, Proline and Hydroxyproline in Varying Sequence and Chain Length Up to n=6, PEG 1000-6,000

(GPHyp)₃ (0.213 g, 0.26 mmol) was dissolved in MeOH (2.5 ml) in glovebox. ⁱPr₂EtN (91 μl, 0.52 mmol) was added and mixture stirred (solution A). HOBT (0.0353 g, 0.26 mmol) and HBTU (0.0992 g, 0.26 mmol) were dissolved in MeOH (12.5 ml) at 40° C., followed by addition of solution A to give suspension B. Suspension B was then added to NBPEGNH₂ (0.25 g, 0.218 mmol) and stirred at r.t. for 24 h. The resulting pale yellow mixture was then concentrated by solvent evaporation to give a beige mixture. The mixture was dispersed into Et₂O and placed in freezer for 48 h. The Et₂O layer was withdrawn and MeOH was added to the residue to give a beige suspension. Beige oily product NB-PEG-(GPHyp)₃ (0.22 g, yield 50%) was obtained upon filtration and solvent evaporation. ¹H NMR (500 MHz, CD₃OD): δ 6.33 (s, 2H), 4.73-4.44 (br, 4H), 3.65 (m, 84H), 3.57 (m, 4H), 3.18 (s, 2H), 2.72 (s, 2H), 2.39-1.80 (br, 8H), 1.44-1.37 (dd, 2H).

### Synthesis of N-(Carboxypentyl)-cis-5-norbornene-exo-2,3-dicarboximide (NCP)

cis-5-norbornene-exo-2,3-dicarboxylic anhydride (4.0 g, 24.3 mmol) and 6-aminohexanoic acid (3.3 g, 25.3 mmol) were weighed into a round-bottom flask. To the solid mixture was added toluene (50 mL) and Et₃N (410 μL, 2.92 mmol). The flask was fitted with a Dean-Stark trap and heated to reflux for 4h. The mixture was then allowed to cool to room temperature and diluted with CH₂Cl₂ (50 mL) and washed with 1 M aqueous HCl (2×20 mL). The organic layer was washed with saturated aqueous NaCl (20 mL), dried (Na₂SO₄), filtered, and concentrated under reduced pressure to provide NCP as a pale yellow solid. ¹H NMR (500 MHz, CD₃OD, 25° C.) δ 6.26 (t, 2H, J=2.0 Hz), 3.44 (m, 2H), 3.25 (m, 2H), 2.66 (d, 2H, J=1.0 Hz), 2.32 (t, 2H, J=7.2 Hz), 1.63 (m, 2H), 1.55 (m, 2H), 1.46-1.51 (m, 1H), 1.33 (m, 2H), 1.19 (d, 1H).

### Synthesis of NCP-PA6 Macromonomer by ROP

NCP-PA6 macromonomers with different degree of polymerization (DP) were prepared by ROP. As an example, ε-caprolactam (2.56 g, 12 mmol) was weighed into a 50 ml rbf containing NCP initiator (0.2 g, 0.6 mmol) under positive N₂ pressure. Deionized H₂O (5 ml) with H₃PO₃ (0.081 g) were added to the mixture and the resultant mixture was heated at 170° C. for 30 min and maintained at 240° C. for 4 h. H₂O was removed by distillation and the reaction was heated at 240° C. under vacuum for another 2 h. A beige solid was precipitated out from MeOH, filtered and the residue washed repeatedly with MeOH to yield NCP-PA6 upon drying overnight in a vacuum oven. ¹H NMR [500 MHz, DCO₂D/CD₂Cl₂ (1:4)]: δ 6.42 (br, PA 6), 6.28 (s, 2H, NCP), 3.42 (s, 2H, NCP), 3.14-3.12 (m, PA 6), 2.67 (s, 2H, NCP), 2.14-2.12 (m, PA 6), 1.56-1.53 (m, PA 6), 1.46-1.44 (m, PA 6), 1.29-1.25 (m, PA 6).

### Typical Procedure for ROMP of NCP-PA6 Macromonomer and NBPEG₃₄₀₀DGEA Macromonomer as Representative Prep for PA-Peptide Type Copolymers

NBPEG₃₄₀₀DGEA macromonomer (0.2 eq.) was weighed into a 4 ml glass vial followed by addition of NCP-PA6 (0.12 g). CH₃CO₂H (0.021 M wrt. NCP-PA6) was added and the mixture stirred at 80° C. till a clear solution is obtained. A solution of catalyst 2 in CH₂Cl₂ (1.25 mol %, 0.05 M) was added to the solution and the mixture is stirred for 24 h at 80° C. Ethyl vinyl ether is added to the reaction followed by MeOH. The mixture was placed in the freezer for 1 day to give a beige ppt. The suspension was centrifuged and mother liquor was decanted. The residue was washed repeatedly with MeOH followed by drying in vacuum oven to give a beige solid product of PA6-DGEA copolymer. ¹H NMR [DCO₂D/CD₂Cl₂(1:4), 500 MHz, 25° C.]: δ 6.42 (br, PA6), 3.60 (s, PEG), 3.19-3.13 (m, PA6), 2.15-2.12 (m, PA 6), 1.62-1.56 (m, PA6), 1.48-1.42 (m, PA6), 1.29-1.23 (m, PA6).

### Example 9: Bioactive Synthetic Copolymers Examples—Antibiotic-Containing Polystyrene for Use in Tissue and Serum Handling Devices

Brush polymers containing pegylated antibiotics and polystyrene were created for use as antimicrobial additives in medical use polystyrene to create non-leachable antibiotic-containing tissue handling devices such as tissue culture plates and serum tubes. Such devices are typically made of medical grade polystyrene and antibiotics are usually added to the medium where the tissue or serum is held in, or coated on the device, which tends to be a costlier approach.

This example reports the development of antibiotic-containing polystyrene for use in tissue and serum-handling devices such as tissue culture plates and serum sample tubes.

Polystyrene (PS) is the synthetic polymer of choice in this example due to its low cost and ease of sterilization by common sterilization techniques such as ethylene oxide, UV and gamma irradiation. PS especially, is very stable towards gamma and e-beam irradiation, amongst other common medical device polymers, making it a very popular material for tissue handling devices since these two sterilization techniques are the most effective methods for sterilization prior to use. Furthermore, the high clarity in the polymer allows its use in tissue and serum handling devices to enable visual inspection of contents from exterior of device.

In this strategy, antibiotics is tethered on a polyethylene glycol (PEG) chain that bears a norbornene-exo-dicarboximide (NB) moiety to create a biomacromonomer, followed by ROMP with a polystyrene-bearing norbornene-exo-dicarboximide synthetic macromonomer, to create that eventual antibiotic-containing polystyrene bioadditive. This bioadditive can then be blended into base medical grade polystyrene for device fabrication.

In penicillin class of antibiotics, the mechanism of action is in the β-lactam ring where the ring binds to the enzyme transpeptidase, preventing the bacteria from forming crosslinks in its cell walls. Crosslinking in peptidoglycans is required for cell wall formation in bacteria cells. By inhibiting cell wall production, bacteria cells die rapidly. Hence, the β-lactam ring of penicillin should be left exposed to bacteria cells for an anti-bacterial effect. Penicillin was chosen to tether via its carboxylic acid terminal, which is fairly distant from the β-lactam ring, thus allowing its reach to bacteria cells. On contact of bacteria cells with the penicillin containing polystyrene, the cells bind to penicillin and its cell walls break as a result of this contact, thus killing the bacteria cells.

Ciprofloxacin (CIF) is a fluoroquinolone-based broad spectrum antibiotic, especially active against gram negative bacteria such as P. aeruginosa. The fluoroquinolone ring binds to DNA gyrase, an essential bacteria enzyme, preventing bacteria cells from replicating. By tethering CIF via its carboxylic terminal and exposing its fluoroquinolone ring in the side arms of the brush polymer (Scheme 3.2), CIF binding sites are allowed to be available for bacteria cell binding, when the cells comes into contact with the polymer surface, thereby killing the bacteria cells present in the sample containers.

Aminoglycosides are broad-spectrum antibiotics that are commonly used as anti-infectives in clinical settings. Such antibiotics are bactericidal and contain hydrophilic saccharide units bearing multiple hydroxy and amino functionalities. Antibiotics that are aminoglycoside-based include streptomycin, ribostamycin and gentamicin. These can be connected to the NBPEG moiety via the —CH₂OH (strep), —CH₂NH₂ (rib) or —CH(CH₃)NH₂ (gen) group on the antibiotic molecule, leaving the binding sites on the molecule exposed to bacteria cell binding. Such antibiotics bind to bacteria ribosomal subunit, preventing them from synthesizing essential proteins for growth. Polymers bearing these antibiotics are useful for tissue culture devices apart from penicillin, since they are part of the standard antibiotic recipe for cell culture media. Once the antibiotic-bearing macromonomer is synthesized, they can be copolymerized using ROMP techniques, with a polystyrene-bearing macromonomer, to create the desired brush polymer of polystyrene and antibiotic, held together by a norbornene dicarboximide backbone (Scheme 9). This antibiotic-containing polystyrene brush polymer is then used as a bioadditive for blending in base medical grade polystyrene for medical device fabrication.

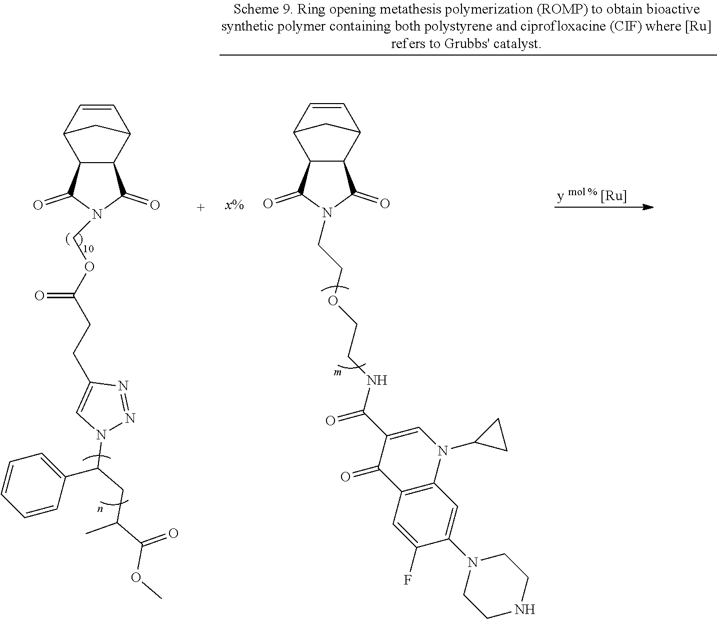

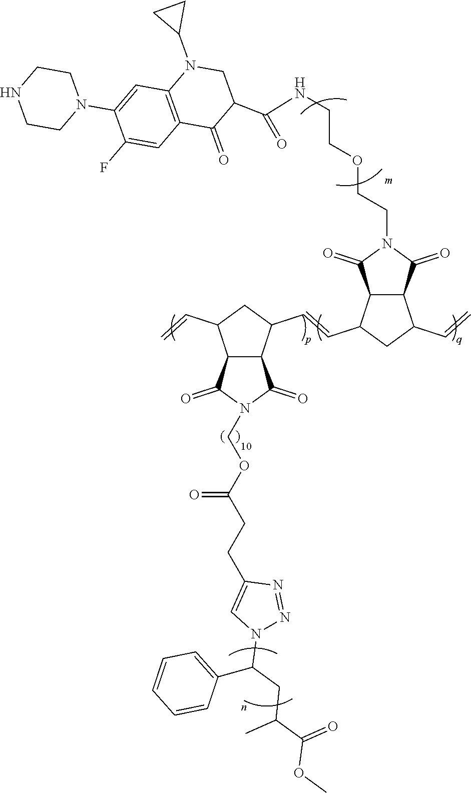

In summary, antibiotic bonded polystyrene copolymer is developed for the creation of antibiotic containing polystyrene tissue handling devices where the antibiotics are covalently bonded to polystyrene and cannot leach out from the material. This is achieved by using ring opening polymerization metathesis technologies on pegylated antibiotics on norbornene dicarboximide linkers and polystyrene on norbornene dicarboximide linkers. The result is a brush polymer with pendant pegylated antibiotics and polystyrene where the active group on the antibiotic molecule is exposed to bacteria cell binding, for bactericidal effects.

### Experimental Procedures

### General procedure

Ring opening metathesis polymerization (ROMP) reactions and bioactive macromonomer syntheses were carried out in a Vacuum Atmosphere glovebox under nitrogen atmosphere. NBPEG and NBPS syntheses were carried out in a fumehood under atmospheric conditions, following procedures provided in Example 6. All solvents used in the glovebox are anhydrous and used as purchased. Grubbs second generation catalyst was purchased from Sigma Aldrich and peptides were purchased from Biomatik Inc. PEG diamine was purchased from Alfa Aesar (1,000 and 3,400) or Sigma Aldrich (6,000). Amoxicillin, Ciprofloxacin, Ribostamycin, HOBT, HBTU, ⁱPr₂EtN were purchased from Sigma Aldrich and cis-norbornene-exo-2,3-dicarboxylic anhydride was purchased from Alfa Aesar. All purchased reagents were used without further purification.

¹H NMR spectra were recorded on a JEOL 500 MHz NMR spectrometer using MeOD as solvent for all biomolecule-based macromonomers. CDCl₃ is used as solvent for PS macromonomer. Gel Permeation Chromatography was carried out on a Waters Aquity APC System equipped with Acquity APC XT 45, XT 200 and XT 450 columns, Acquity RI detector. THF was used in sample preparation and a flow rate of 1.0 ml/min at 40° C. was used.

Synthesis of NBPEG and NB—PS are described in Example 6.

### Synthesis of NBPEG₁₀₀₀CIF as representative prep for PEG 1,000-6,000

Ciprofloxacin (0.0866 g, 0.26 mmol), was suspended in MeOH (2.5 ml) in a 4 ml vial, in the glovebox. ⁱPr₂EtN (91 μL, 0.52 mmol) was added and the mixture stirred (A). HOBT (0.0353 g, 0.26 mmol) and HBTU (0.0992 g, 0.26 mmol) were dissolved in MeOH (12.5 ml) in a 20 ml vial at 40° C., followed by addition of the CIF solution from (A), to give suspension (B). Suspension B is then added to NBPEG₁₀₀₀ (0.25 g, 0.218 mmol) in a 40 ml vial and the mixture stirred at room temperature overnight to give a pale yellow solution with white suspension. The mixture was then evaporated to dryness and the oil was added to diethylether (50 ml). The diethylether solution was chilled in a freezer for 48 h and decanted to give a white sticky residue. MeOH (3 ml) was added to the residue and the mixture was added to diethyl ether in an Erlenmeyer flask which was again placed in freezer for another 48 h to give a yellow oil at the bottom of flask. The ether was decanted and MeOH (3 ml) was added to give a yellow solution with white ppt. The mixture was passed through a syringe filter and the clear filtrate was evaporated to dryness to give a yellow oil of NBPEGCIF. ¹H NMR (500 MHz, MeOD): δ=7.76 (ddd, 2H), 7.44-7.37 (m), 6.36 (t, 2H), 3.67 (br s, 82H), 3.21 (t, 2H), 2.75 (d, 2H), 2.73 (s, 5H), 1.25 (d, 1H).

### Representative Synthesis for PS-Antibiotic Polymer by ROMP Using NBPEGCIF as Example

NBPEG₁₀₀₀CIF macromonomer (0.1 eq) was weighed into a 4 ml glass vial followed by addition of NB-PS (0.050 g, 0.030 mmol). THF (0.05 M wrt. NB-PS) was added and the mixture was stirred at r.t. till a clear solution is obtained. A solution of catalyst 2 (referred to in Example 5) in THF (1.25 mol %, 0.05 M) was added to the solution and the mixture was stirred for 2 h at r.t. before the reaction was terminated by adding ethyl vinyl ether. The polymer solution was precipitated in Methanol. The polymer mixture was centrifuged and supernatant was decanted. The residue was washed repeatedly with MeOH followed by drying under vacuum to give a white powdered polymer. ¹H NMR (500 MHz, CDCl₃): δ 6.99-7.15 (m, 3H, Ph), 6.4-6.8 (m, 2H, Ph), 3.60 (s, 4H, PEG), 1.93 (quintet, 1H, PS), 1.42 (t, 2H, PS). GPC analysis (THF): Mₙ=30,056, PDI=1.36

### Example 10: Bioactive Synthetic Copolymers Examples—Polylactide-Biomolecule Copolymers as Bioadditives for Human Skin and Bone Tissue Regeneration

A series of brush copolymers containing polylactide (PLA) side chains and pegylated biomolecules including integrin binding peptides, collagen mimics or fragments (COL) and glycosaminoglycans (GAGs), were synthesized via ring-opening metathesis polymerization (ROMP). These copolymers can be utilized as bioadditives in scaffold materials for either human skin or bone tissue regeneration.

Biomolecules used in this example include heparin oligosaccharide (HS) DP12, DP14, integrin binding peptide such as RGD, collagen fragments with repeating units of glycine, proline and hydroxyproline (G, P, Hyp) in varying sequences and length, and collagen mimic DGEA.

Polylactide (PLA) is the synthetic polymer of choice in this example as it degrades under physiological conditions to form non-toxic lactic acid which is also present in the human body, i.e. bioresorbable polymer. Due to its biocompatibility and good processability, PLA and its copolymers are commonly being used in medical implants and tissue engineering. Bioactive molecules which can promote skin or bone tissue regeneration are then incorporated into the final polymer via copolymerization in our approach.

Integrin-binding peptides such as RGD (Arg-Gly-Asp) which are found in several extracellular matrix proteins have been identified as an important motif for cell recognition and cell adhesion. The immobilization of RGD onto scaffolds has been shown to enhance cell attachment, migration and proliferation. RGD can also promote osteogenic differentiation and mineralization, thereby inducing bone regeneration. Due to the hygroscopic nature of RGD, it is difficult to handle and administer by itself. Therefore, by covalently attaching the peptide onto the synthetic PLA polymer, it will increase its stability and the ease of handling. These RGD-containing polymers can be subsequently blended with base material to create scaffolds for skin or bone regeneration.

In addition to RGD peptides, polymers containing collagen mimics or fragments have also been synthesized. Collagen is the most abundant protein in the extracellular matrix and has been widely used in biomaterials to increase biocompatibility and encourage tissue regeneration. However, full-length human collagen requires complex synthesis and often show poor solubility in buffers. Short collagen-mimic peptide sequences or fragments which include crucial peptide sequences at a fraction of the length has been used to elicit similar biological response to their full-length collagen counterparts. Collagen mimics such as DGEA (Asp-Gly-Glu-Ala) and collagen fragments bearing varying lengths of glycine, proline and hydroxyproline sequences are incorporated into the synthetic polymer. Without being bound by theory, it is believed that DGEA promotes cell adhesion, spreading and osteogenic differentiation which will be advantageous for applications in both skin and bone regeneration.

Heparin sulfate (HS) is a GAG having repeating disaccharide units that have been heavily modified with sulfate groups. Without being bound by theory, it is believed that HS chain of between 5 to 10 disaccharide units is the most active towards binding of bone morphogenetic proteins (BMP). Without being bound by theory, it is believed that HS directly regulates BMP-2-mediated differentiation of myoblasts onto osteoblasts. In particular, the HS fragment with hexa-disaccharide unites (DP12) is believed to have the highest binding affinity for BMP-2. In vitro studies of BMP-2 complexed with DP12 demonstrated enhanced osteogenic differentiation in cells while in vivo work using rat models revealed improved bone tissue regeneration using DP12 as compared to controls of collagen sponge in polycaprolactone (PCL) tubes. HS interacts with angiogenic factors and induce vascularization. Vascularization is critical in tissue scaffolds to deliver oxygen and nutrients throughout the engineered tissue. In addition, HS can interact with growth factors that stimulates epithelial repair and encourage wound healing. PLA with HS molecules such as DP12 or DP14 incorporated would be desired for use in scaffold materials to allow for skin or bone tissue regeneration. However, HS molecules are highly hygroscopic and cannot be simply coated onto the polymer. The high aqueous solubility of HS also means that it may leach into the body and not remain on the desired site where tissue regeneration is required. In the present approach, macromonomers containing HS molecules are copolymerized with PLA macromonomer to prepare the bioactive copolymer which will be blended with base polymer PLA for skin scaffold or bone implant fabrication. This will ensure the HS molecules will be localized on the implant site and not induce undesirable effects in other parts of the body.

The final brush copolymers are prepared by ROMP (Scheme 10) using Grubbs type catalyst via copolymerization of PLA macromonomer with bioactive macromonomer.

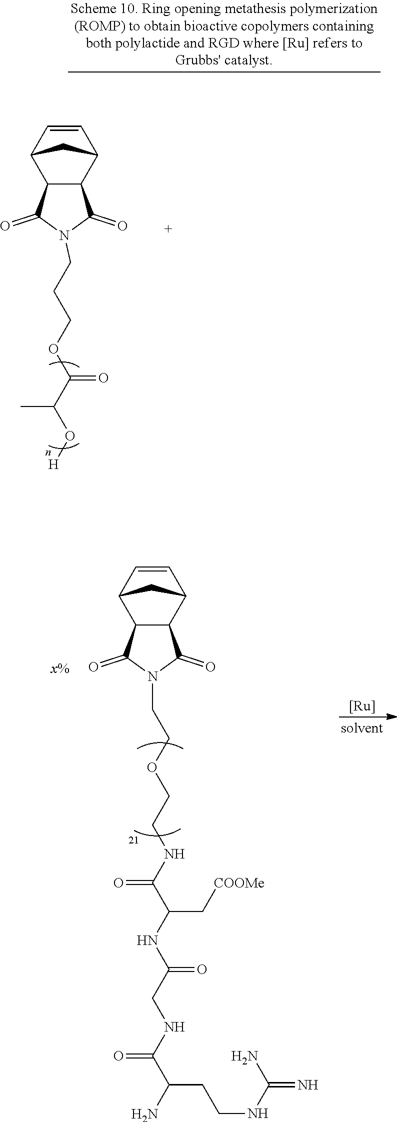

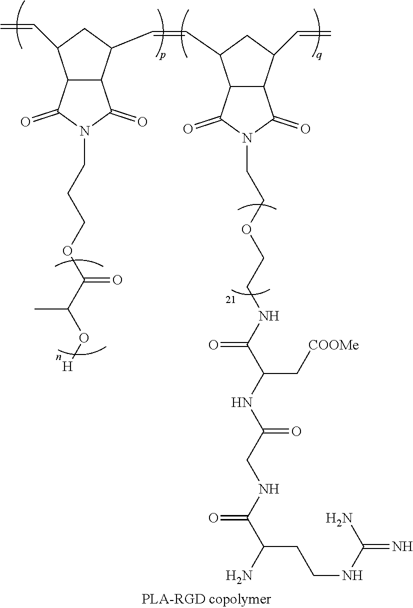

### Biocompatibility

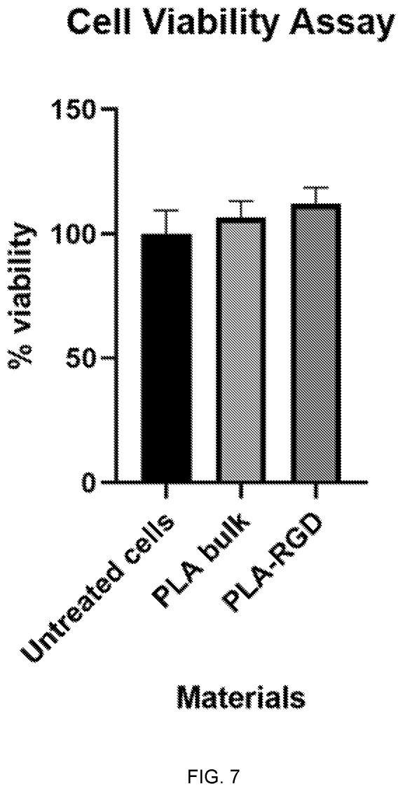

To demonstrate the biocompatibility of the polymers, the materials were tested on human fibroblast cells in vitro. The bioactive synthetic polymer (PLA-RGD) was blended with commercial PLA as base material and electrospun into thin sheets. Commercial base polymer PLA (PLA-bulk) was used as control for this study. The sheets were then tested on human fibroblasts Hs27 and all tested materials showed good biocompatibility with high cell viability after 72 h (FIG. 7). From the preliminary data, >100% cell viability of the bioactive synthetic polymer designed in accordance with various embodiments disclosed herein was observed, indicating cell proliferation (cell growth) versus cell death (<100%). This demonstrates low toxicity of the materials to human fibroblasts. Furthermore, bioactive polymer-containing PLA showed improvement in cell viability over base polymer PLA, indicating their ability to enhance biocompatibility of pure PLA itself. Optimization of the biomolecule concentration in the bioactive synthetic polymer and blending ratios are ongoing to obtain the best tissue regeneration outcome for this material.

In summary, a series of brush copolymers that have biodegradable PLA side chains and bioactive molecules such as integrin binding peptides, collagen mimics or fragments (COL) and heparin sulfate (HS) were synthesized. These bioactive polymers can be blended with base material, such as medical grade PLA, to create scaffold materials for use in skin or bone regeneration.

### Experimental Procedures

### General Procedure

Ring opening metathesis polymerization (ROMP) reactions and bioactive macromonomer syntheses were carried out in a Vacuum Atmosphere glovebox under nitrogen atmosphere. PLA macromonomer (NPH-PLA) synthesis were carried out using standard Schlenk line techniques under nitrogen atmosphere. NBPEG and NPH synthesis was carried out in a fumehood under atmospheric conditions, following procedures provided in Example 6. All solvents used in the glovebox are anhydrous and used as purchased. Grubbs second generation catalyst was purchased from Sigma Aldrich and peptides were purchased from Biomatik Inc. Catalyst 2 ((H₂IMes)(pyr)₂(Cl)₂RuCHPh) was synthesized according to procedure provided in Example 6. PEG diamine were purchased from Alfa Aesar (1,000 and 3,400) or Sigma Aldrich (6,000). HOBT, HBTU, ⁱPr₂EtN were purchased from Sigma Aldrich and cis-norbornene-exo-2,3-dicarboxylic anhydride was purchased from Alfa Aesar. All purchased reagents were used without further purification. Heparin oligosaccharides DP12 and DP14 were purchased from Iduron.

¹H NMR spectra were recorded on a JEOL 500 MHz NMR spectrometer using MeOD or D₂O as solvent for all biomolecule-based macromonomers. CDCl₃ was used as solvent for PLA macromonomer and ROMP polymers. Gel Permeation Chromatography was carried out on a Waters Aquity APC System equipped with Acquity APC XT 45, XT 200 and XT 450 columns, Acquity RI detector. THF was used in sample preparation and a flow rate of 1.0 ml/min at 40° C. was used.

Synthesis of NBPEG, NBPEGRGD, NBPEG(DGEA), NBPEG(GPHyp)₃ and NBPEGDP12 are described in Example 6.

### Synthesis of NPH-PLA Macromonomer by ROP

NPH-PLA macromonomers with different degree of polymerization (DP) were prepared by ROP. As an example, a 25 mL Schlenk tube was charged with NPH initiator (110 mg, 0.50 mmol), D, L-lactide (864 mg, 6.0 mmol), Sn(Oct)₂ (2 mg), and a stir bar. The tube was evacuated and backfilled with nitrogen four times, and was then immersed in an oil bath at 130° C. After 2.5 h, the contents were cooled to room temperature, diluted with dichloromethane, and precipitated into cold MeOH twice. The mother liquor was decanted and the residue washed with MeOH, followed by drying in vacuum oven.

¹H NMR (CDCl₃, 500 MHz): δ 6.28 (br t, 2H), 5.27-5.08 (m, PLA), 4.35 (m, 1H), 4.19-4.02 (m, 2H), 3.62-3.44 (m, 2H), 3.27 (s, 2H), 2.69 (m, 2H), 1.97-1.47 (m, PLA), 1.19 (d, 1H).

GPC analysis (THF): Mₙ=2,471, PDI=1.20, yield 0.600 g.

Synthesis of NBPEG₃₄₀₀DP14

DP14 (0.0285 g, 8.4 μmol) was dissolved in MeOH/DMF (0.5 ml/1.0 ml) in an 8 ml scintillation vial. ⁱPr₂EtN (2.9 μl, 16.8 μmol) was added and mixture stirred (solution A). HOBt (0.0011 g, 8.4 μmol) and HBTU (0.0032 g, 8.4 μmol) were dissolved in MeOH (2.5 ml) at 40° C., followed by addition of the solution A to give suspension B. Suspension B was then added to NBPEG₃₄₀₀NH₂ (0.025 g, 7.03 μmol) and stirred at r.t. for 24 h. The resulting pale yellow mixture was centrifuged to remove insoluble impurities. Supernatant was concentrated with solvent evaporation and then precipitated into cold Et₂O and the mixture was placed in freezer for 24 h. Orange powder product (43.5 mg, yield 90%) was obtained upon filtration.

¹H NMR (500 MHz, D₂O): 6.36 (t, 2H), 4.30 (br, 4H), 3.70 (s, 340H), 2.85 (s, 2H), 2.71 (s, 2H), 1.40-1.32 (m, 10H).

### Typical Procedure for ROMP of NPH-PLA Macromonomer and NBPEG₁₀₀₀RGD Macromonomer as Representative Preparation for PLA-Peptide Type Copolymers

NBPEG₁₀₀₀RGD macromonomer (0.1 eq.) is weighed into a 4 ml scintillation vial followed by addition of NPH-PLA (0.05 g). THF (0.05 M wrt. NPH-PLA) is added and the mixture stirred at r.t. till a clear solution is obtained. A solution of catalyst 2 in THF (1.25 mol %) is added to the solution and the reaction is stirred for 1 h. Ethyl vinyl ether is added to the reaction mixture followed by MeOH (3 ml) and the mixture placed in the freezer for 1 h to give a sticky solid. The mother liquor was decanted and the residue washed repeatedly with MeOH followed by drying in vacuum oven.

For PLA-RGD copolymer, ¹H NMR (500 MHz, CDCl₃): δ 5.27-5.08 (m, PLA), 3.60 (s, PEG), 1.97-1.47 (m, PLA). GPC analysis (THF): Mₙ=76,700, PDI=1.44.

### Typical Procedure for ROMP of NPH-PLA Macromonomer and NBPEG₁₀₀₀(GPHyp)₃ Macromonomeras Representative Preparation for PLA-COL Type Copolymers

NBPEG₁₀₀₀(GPHyp)₃ macromonomer (0.1 eq.) is weighed into a 4 ml scintillation vial followed by addition of NPH-PLA (0.05 g). THF (0.05 M wrt. NPH-PLA) is added and the mixture stirred at 45° C. A solution of catalyst 2 in THF (1.25 mol %) is added to the solution and the reaction is stirred at 45° C. for 2 h. Ethyl vinyl ether is added to the reaction mixture followed by MeOH (3 ml) and the mixture placed in the freezer for 1 h to give sticky solid. The mother liquor was decanted and the residue washed repeatedly with MeOH followed by drying in vacuum oven. ¹H NMR (500 MHz, CDCl₃): δ 5.27-5.08 (m, PLA), 3.60 (s, PEG), 1.97-1.47 (m, PLA). GPC analysis (THF): Mₙ=97,262, PDI=1.14.

### Typical Procedure for ROMP of NPH-PLA Macromonomer and NBPEG₃₄₀₀DP14 Macromonomer as Representative Preparation for PLA-HS Type Copolymers

NBPEG₃₄₀₀DP14 macromonomer (0.1 eq.) is weighed into a 4 ml scintillation vial followed by addition of NPH-PLA (0.03 g). THF (0.02 M wrt. NPH-PLA) is added and the mixture stirred at 45° C. A solution of catalyst 2 in THF (1.25 mol %) is added to the solution and the reaction is stirred at 45° C. for 2 h. Ethyl vinyl ether is added to the reaction mixture followed by MeOH (3 ml) and the mixture placed in the freezer for 1 h to give a sticky solid. The mother liquor was decanted and the residue washed repeatedly with MeOH followed by drying in vacuum oven.

¹H NMR (500 MHz, CDCl₃): δ 5.27-5.08 (m, PLA), 3.60 (s, PEG), 1.97-1.47 (m, PLA).

### Example 11: Bioactive Synthetic Copolymers Examples—PLGA-Peptide and -Oligosaccharides Polymers for Cartilage Tissue Regeneration

A series of poly(lactic-co-glycolic acid) (PLGA) peptide and oligosaccharide brush polymers were prepared by ring opening metathesis polymerization. Extracellular matrix (ECM) peptides such as RGD, collagen fragments and oligosaccharides such as heparin oligosaccharides, have been pegylated and linked to PLGA, as side chains, on a poly(norbornene-exo-2,3-dicarboximide) backbone, via ring opening metathesis polymerization reactions. The resultant brush polymers can be used as bioadditives for cartilage tissue regeneration materials that are PLGA-based. Preliminary in vitro tests on the bioactive PLGA demonstrated excellent cell viability with some degree of cell proliferation at 72 h.

Biomolecules used in this example include ECM peptides such RGD, collagen fragments and collagen mimics that are known to regenerate cartilage tissues. The synthetic polymer of choice in this example is PLGA, a bioresorbable polymer that has properties between that of PLA and poly(glycolic acid) (PGA). The overall polymer created is shown to be thermally stable and bioactive polymer that is osteoinductive for use in cartilage implants.

Using the bioactive synthetic polymer technology in accordance with various embodiments disclosed herein, collagen-bearing synthetic polymers are created that allow collagen to be introduced to synthetic materials without loss in functionality of these collagen fragments. Furthermore, the PLGA side chains in these bioactive synthetic polymer helps increase the thermal stability of collagen and allows efficient blending of an otherwise hygroscopic collagen into base polymer PLGA, which is hydrophobic. The overall PLGA material is not only bioactive but thermally stable and mechanically strong, for material processing and use in meniscal cartilage implants.

PLGA is chosen as the synthetic polymer due to its better control of polymer crystallinity, melting point and load-bearing capabilities, over its homopolymer counterpart, PGA and PLA where PGA is more crystalline and higher melting than PLA. However, PLGA is non-osteoinductive despite its apparent biocompatibility. Hence, there is a need to introduce a stimulus on PLGA by copolymerizing it with bioactive macromonomers that are osteoinductive or osteoconductive. The overall material is then a mechanically strong, thermally stable, osteoinductive polymer, for use in cartilage implants. PLGA is also biodegradable, allowing the patient's own cartilage to take over the synthetic material, after the material degrades in the body. The by-products of the polymer are lactic acid and glycolic acid, both of which are non-toxic to human.

To enhance the softness of the material and its hydrophilicity, polyethylene glycol (PEG) is introduced into the bioactive synthetic polymer chain. This same strategy is used in tuning the softness/hardness of material. By adjusting PEG to PLGA side chain content in the bioactive synthetic polymer, as well as the PLGA-COL bioactive synthetic polymer ratio to PLGA base material, overall hardness of the material can be adjusted. This is especially important in articular cartilage implant. To further enhance the strength of the articular cartilage implant, lattice designs in additive manufacturing (AM) of the scaffold may be used/created. Material strength can be greatly enhanced through lattice designs using AM, whilst retaining porosity of material for enhanced osseointegration in scaffold, and lightweight of entire scaffold.

Apart from ECM peptides, heparin sulfate mimics such as highly sulfated glycosaminoglycans, can also be used to provide necessary stimulus required for articular cartilage regeneration. Glycosaminoglycans (GAGs) are heterogeneous polysaccharides ubiquitously found in mammalian tissues, and heparin sulfate (HS) is a highly sulfated GAG with enormous structural diversity that can interact with a plethora of proteins to regulate many physiological processes. Proteins that interact with HS include growth factors (GF), chemokines, enzyme inhibitors, extracellular matrix proteins and membrane-bound receptors. HS potentiates key GFs responsible for cell proliferation and differentiation, including bone morphogenetic protein BMP-2, which is important in bone growth, as well as vascular endothelial growth factor (VEGF) and fibroblast growth factor (FGF), which is important for blood vessel formation. By incorporating such oligosaccharides into PLGA, BMP binding properties are introduced to the polymer and the material can be used in cartilage implants. Through the combination of material design by advance chemical syntheses and scaffold design fabrication by additive manufacturing, different types of bone scaffolds are created for regeneration of different parts of the bone, depending on need. Alternatively, the scaffolds can also be fabricated by other material processing methods such as melt extrusion, injection molding and electrospinning.

With both the biomacromonomer and synthetic macromonomer, the final bioactive synthetic polymer is prepared by ROMP using Grubbs type catalysts (Scheme 11).

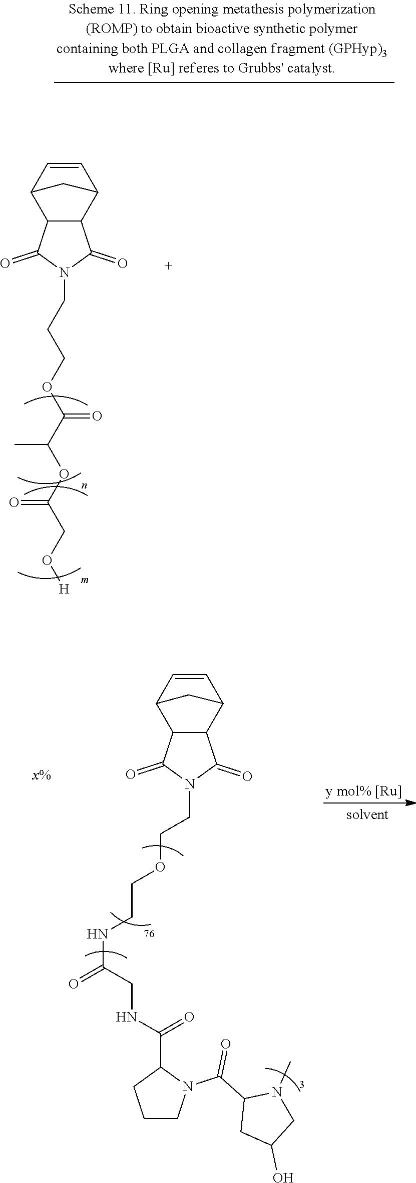

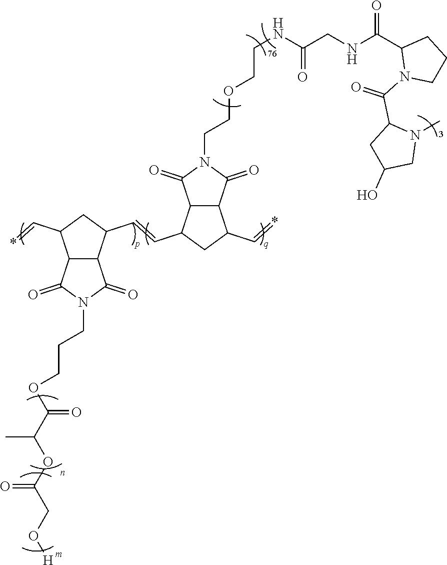

Upon characterization of bioactive polymers, TG-DSC analysis to ascertain melting point and degradation temperature of polymer is carried out. ICP-MS to ensure metal residues from ruthenium catalysts have been reduced to a minimal, below ISO10993 guidelines for metal catalysts in biomedical devices, is also carried out before material processing. Once these parameters have been ascertained, the materials can be processed into prototypes for in vitro testing to ascertain biocompatibility of material and cell viability.

### Biocompatibility

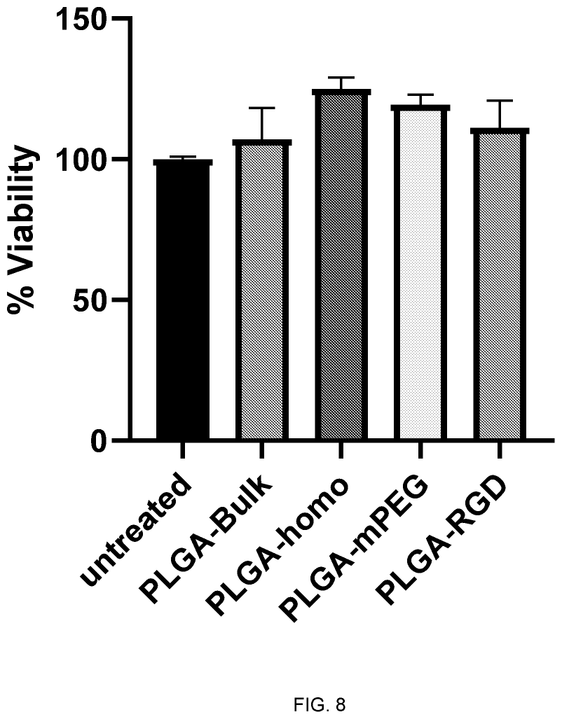

To demonstrate the biocompatibility of the polymers, the materials on human fibroblast cells were tested in vitro. The bioactive synthetic polymer (PLGA-RGD) was blended with commercial PLGA as base material and electrospun into thin sheets. Commercial base polymer PLGA (PLGA-Bulk), PLGA ROMP homopolymer (PLGA-homo) and PLGA-mPEG₅₀₀₀ were used as controls for this study. The sheets were then tested on human fibroblasts Hs27 and all tested materials showed good biocompatibility with high cell viability after 72 h (FIG. 8). From the preliminary data, it can be seen that there is >100% cell viability of the bioactive synthetic polymer designed in accordance with various embodiments disclosed herein, indicating cell proliferation (cell growth) versus cell death (<100%). This demonstrates low toxicity of the materials to human fibroblasts. Furthermore, all bioactive polymer-containing PLGA showed improvement in cell viability over base polymer PLGA, indicating their ability to enhance biocompatibility of pure PLGA itself. Optimization of the biomolecule concentration in the bioactive synthetic polymer and blending ratios to obtain the best tissue regeneration outcome for this material are ongoing.

In summary, a series of biodegradable polymers that bear biodegradable synthetic polymer side chains of PLGA and bioactive side chains of extracellular matrix peptides or sulfated glycosaminoglycans were developed, for cartilage tissue regeneration. The materials can be processed by a wide variety of material processing methods such as melt extrusion, FFF or FDM type 3D-printing and electrospinning. Preliminary in vitro tests have demonstrated good cell viability and proliferation without the introduction of stem cells or growth factors.

### Experimental Procedures

### General Procedure

Ring opening metathesis polymerization (ROMP) reaction and bioactive macromonomer syntheses were carried out in a Vacuum Atmosphere glovebox under nitrogen atmosphere. PLGA macromonomer (NPH-PLGA) synthesis were carried out using standard Schlenk line techniques under nitrogen atmosphere. NBPEG and NPH synthesis was carried out in a fumehood under atmospheric conditions, following procedures provided in Example 6. All solvents used in the glovebox are anhydrous and used as purchased. Grubbs second generation catalyst was purchased from Sigma Aldrich and peptides were purchased from Biomatik Inc. PEG diamine was purchased from Alfa Aesar (1,000 and 3,400) or Sigma Aldrich (6,000). HOBT, HBTU, ⁱPr₂EtN were purchased from Sigma Aldrich and cis-norbornene-exo-2,3-dicarboxylic anhydride was purchased from Alfa Aesar. All purchased reagents were used without further purification.

¹H NMR spectra were recorded on a JEOL 500 MHz NMR spectrometer using MeOD as solvent for all biomolecule-based macromonomers. CDCl₃ is used as solvent for PLGA macromonomer. Gel Permeation Chromatography was carried out on a Waters Aquity APC System equipped with Acquity APC XT 45, XT 200 and XT 450 columns, Acquity RI detector. THF was used in sample preparation and a flow rate of 1.0 ml/min at 40° C. was used.

Synthesis of NBPEG and NBPEG(peptide) are described in Example 6.

### Synthesis of NPH(PLGA) Macromonomer

NPH-PLGA macromonomers with different degree of polymerization (DP) were prepared by ROP. As an example, a 25 mL Schlenk tube was charged with NPH initiator (55 mg, 0.25 mmol), D, L-lactide (864 mg, 6.0 mmol), glycolide (174 mg, 1.5 mmol), Sn(Oct)₂ (2 mg), and a stir bar. The tube was evacuated and backfilled with nitrogen four times, and was then immersed in an oil bath at 125° C. After 3 h, the contents were cooled to room temperature, diluted with dichloromethane, and precipitated into cold MeOH. The mother liquor was decanted and the residue washed with MeOH, followed by drying in vacuum oven.

¹H NMR (500 MHz, CDCl₃): δ 6.28 (br t, 2H), 5.27-5.08 (m, PLA), 4.85-4.65 (m, PGA) 4.35 (m, 1H), 4.19-4.02 (m, 2H), 3.62-3.44 (m, 2H), 3.27 (s, 2H), 2.69 (m, 2H), 1.97-1.47 (m, PLA), 1.19 (d, 1H). GPC analysis (THF): Mₙ=4,336, PDI−1.27.

### Synthesis of NBPEG₁₀₀₀(GPHyp)₃ as Representative Prep for Collagen Fragments of Glycine, Proline and Hydroxyproline in Varying Sequence and Chain Length Up to n=6, PEG 1000, 3,400 and 6,000

(GPHyp)₃ (0.213 g, 0.26 mmol) was dissolved in MeOH (2.5 ml) in glovebox. ⁱPr₂EtN (91 μl, 0.52 mmol) was added and mixture stirred (solution A). HOBT (0.0353 g, 0.26 mmol) and HBTU (0.0992 g, 0.26 mmol) were dissolved in MeOH (12.5 ml) at 40° C., followed by addition of the solution A to give suspension B. Suspension B was then added to NBPEGNH₂ (0.25 g, 0.218 mmol) and stirred at r.t. for 24 h. The resulting pale yellow mixture was then concentrated by solvent evaporation to give beige mixture. The mixture was dispersed into Et₂O and freeze for 48 h. The Et₂O layer was withdrawn and MeOH was added to the residue to give a beige suspension. Beige oily product NB-PEG-(GPHyp)₃ (0.22 g, yield 50%) was obtained upon filtration and solvent evaporation.

¹H NMR (500 MHz, CD₃OD): δ 6.33 (s, 2H), 4.73-.4.44 (br, 4H), 3.65 (s, 84H), 3.57 (m, 4H), 3.18 (s, 2H), 2.72 (s, 2H), 2.39-1.80 (br, 8H), 1.44-1.37 (dd, 2H).

### Representative Synthesis for PLGA-PEG_{3.4K}(GPHyp)₃ Polymer by ROMP Using NPH(PLGA) and NB-PEG_{3.4k}(GPHyp)₃ as Example

NBPEG_{3.4K}(GPHyp)₃ macromonomer (0.1 eq) was weighed into a 4 ml glass vial followed by addition of NPH(PLGA) (0.050, 0.012 mmol). THF (0.05 M wrt. NPH(PLGA) was added and the mixture was stirred at 40° C. till a clear solution is obtained. A solution of catalyst 2 in THF (1.25 mol %, 0.05 M) was added to the solution and the mixture was stirred for 2 h at 40° C. before the reaction was terminated by adding ethyl vinyl ether. The polymer solution was precipitated in methanol. The polymer mixture was centrifuged and supernatant was decanted. The residue was washed repeatedly with MeOH followed by drying under vacuum. The obtained polymer is white powder.

¹H NMR (500 MHz, CDCl₃): δ 5.20 (m, 1H, PLA), δ 4.8 (m, 2H, PGA), δ 3.63 (s, 4H, PEG), δ 1.56 (d, 3H, PLA). GPC analysis (THF): Mₙ=65,166, PDI=1.23.

### Example 12: Bioactive Synthetic Copolymers Examples—PMMA-Peptide Copolymers for Use as Bioadditives in Medical Implants

A series of brush copolymers containing poly(methyl methacrylate) (PMMA) side chains and biomolecules tethered on PEG moieties were synthesized via ring-opening metathesis polymerization (ROMP). Biomolecules may include collagen fragment or collagen mimic peptides from 3-20 amino acid residues in any sequence, such as DGEA, (Gly-Pro-Hyp)₃ and (Pro-Hyp-Gly)₃. These brush polymers can be blended with base polymer PMMA to create bioactive materials for use in biomedical implants such as bone cements, bone implants and craniofacial implants.

This example reports the facile synthesis of brush copolymers containing PMMA side chains and collagen mimics tethered on PEG moieties, for use as bioactive polymers in PMMA-based biomedical implants.

PMMA is the synthetic polymer of choice in this example as it is biocompatible, non-degradable and lightweight thermoplastic with good mechanical strength. It is the first synthetic polymer used in biomedical applications and has now been used in various medical implants such as in intraocular lens, rhinoplasty, dentistry and orthopedics. PMMA is also currently the most widely used alloplastic implant material for craniomaxillofacial reconstructions. PMMA polymers are often modified with varying amounts of additives or fillers to achieve the desired properties in the final materials. PMMA-based implant materials can be fabricated using traditional molding methods such as injection molding or extrusion and also 3D-printing. With the rapid progress in 3D printing technology, PMMA has been increasingly utilized in patient-specific biomedical applications for the fabrication of customized medical implant structures.

In this example, PMMA brush copolymers with collagen fragments or mimics have been synthesized using ROMP. Collagen is the most abundant protein in the extracellular matrix and has been widely used in biomaterials to increase biocompatibility and encourage tissue regeneration. However, full-length human collagen requires complex synthesis and often show poor solubility in buffers. Short collagen-mimic peptide sequences or fragments which include crucial peptide sequences at a fraction of the length may be used to elicit similar biological response to their full-length collagen counterparts. However, as with many biomolecules, these peptides are extremely hygroscopic. Without anchoring the collagen fragments or mimics to a synthetic polymer to increase its ease of handling, it is challenging to create an implant for insertion into human body. Furthermore, PMMA medical implants are foreign materials to the body and can trigger host immune response, leading to inflammation of tissues. PMMA itself also does not support osseointegration of the structure with other structures that it comes in contact with. Therefore, without being bound by theory, it is believed that by incorporating collagen fragments or collagen mimics (COL) in the PMMA polymer, it would help to increase the biocompatibility and biomimetic properties of the material. Some possible collagen mimics used include DGEA (Asp-Gly-Glu-Ala) and collagen fragments bearing varying lengths of glycine, proline and hydroxyproline sequences, in any order. Without being bound by theory, it is believed that DGEA promotes cell adhesion, osteogenic differentiation and osseointegration which will be advantageous for applications in bone or craniofacial implants.

The final brush copolymers are prepared by ROMP using Grubbs type catalyst (Scheme 12).

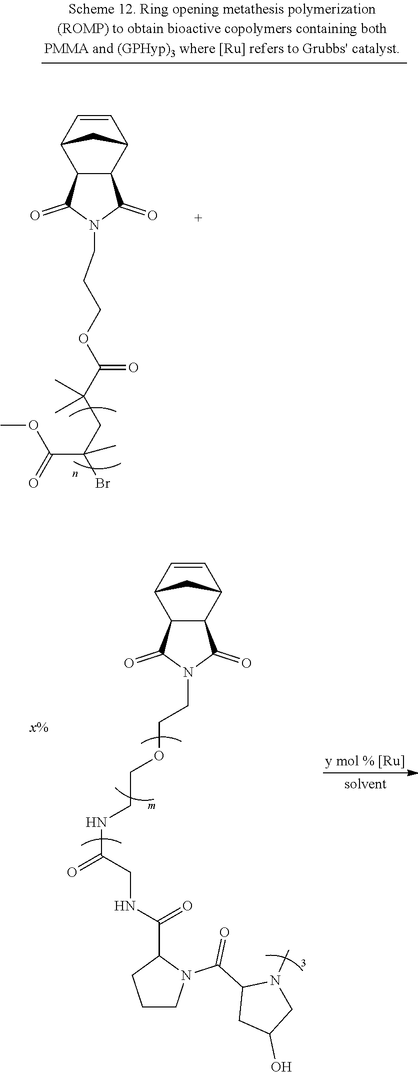

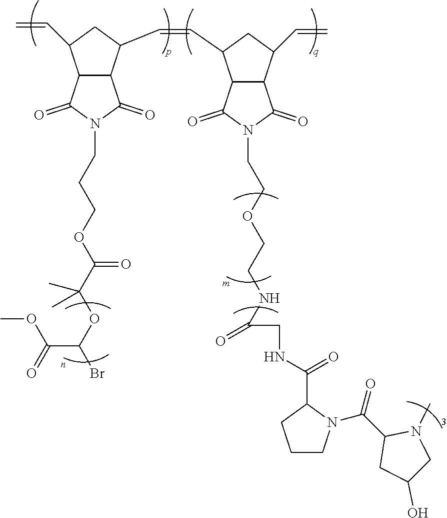

The bioactive PMMA polymers can be blended with medical grade PMMA, and processed by either extrusion, 3D-printing or electrospinning into relevant shapes and tested for biocompatibility.

### Biocompatibility

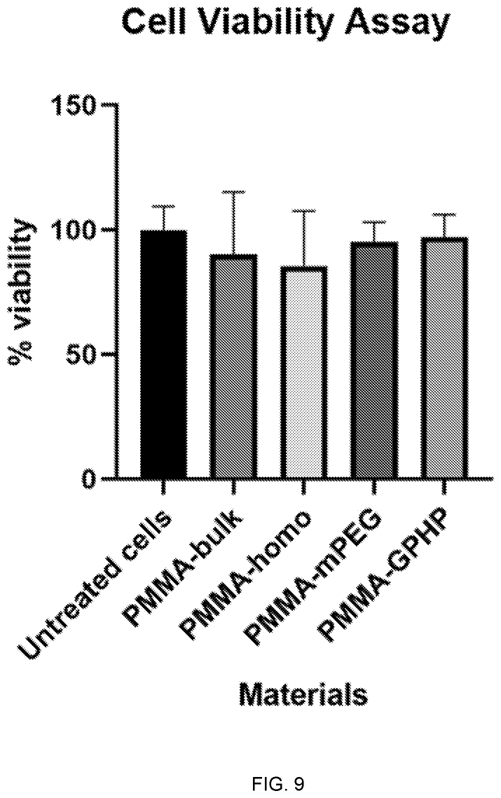

To demonstrate the biocompatibility of the polymers, the materials were tested on human fibroblast cells in vitro. The bioactive synthetic polymer (PMMA-GPHyp) was blended with commercial PMMA as base material and electrospun into thin sheets. Commercial base polymer PMMA (PMMA-bulk), PMMA ROMP homopolymer (PMMA-homo) and PMMA-mPEG₅₀₀₀ were used as controls for this study. The sheets were then tested on human fibroblasts Hs27 and all tested materials showed good biocompatibility with good cell viability after 72 h (FIG. 9). This demonstrates low toxicity of the materials designed in accordance with various embodiments disclosed herein to human fibroblasts. Furthermore, bioactive polymer-containing PMMA showed improvement in cell viability over base polymer PMMA, indicating their ability to enhance biocompatibility of pure PMMA itself. Optimization of the biomolecule concentration in the bioactive synthetic polymer and blending ratios would be performed to improve the biocompatibility of the material, moving forward.

In summary, a series of brush copolymers that have PMMA side chains and peptide molecules such as collagen mimics or fragments (COL) have been synthesized. These bioactive polymers can be used as bioadditives to create implant materials for use in orthopedic or cranioplasty.

### Experimental Procedures

### General Procedure

Ring opening metathesis polymerization (ROMP) reactions and bioactive macromonomer syntheses were carried out in a Vacuum Atmosphere glovebox under nitrogen atmosphere. PMMA macromonomer (NB-PMMA) synthesis were carried out using standard Schlenk techniques under nitrogen atmosphere. NBPEG and norbornenyl-functionalized ATRP initiator synthesis were carried out in a fumehood under atmospheric conditions, following procedures provided in Example 6. All solvents used in the glovebox are anhydrous and used as purchased. Grubbs second generation catalyst was purchased from Sigma Aldrich and peptides were purchased from Biomatik Inc. Catalyst 2 is synthesized according to the procedure provided in Example 6. PEG diamine was purchased from Alfa Aesar (1,000 and 3,400) or Sigma Aldrich (6,000). HOBT, HBTU, ⁱPr₂EtN were purchased from Sigma Aldrich and cis-norbornene-exo-2,3-dicarboxylic anhydride was purchased from Alfa Aesar. All purchased reagents were used without further purification.

¹H NMR spectra were recorded on a JEOL 500 MHz NMR spectrometer using MeOD or D₂O as solvent for all biomolecule-based macromonomers. CDCl₃ was used as solvent for PMMA macromonomer and ROMP polymers. Gel Permeation Chromatography was carried out on a Waters Aquity APC System equipped with Acquity APC XT 45, XT 200 and XT 450 columns, Acquity RI detector. THF was used in sample preparation and a flow rate of 1.0 ml/min at 40° C. was used.

Synthesis of NBPEG, NBPEG(DGEA) and NBPEG(GPHyp)₃ are described in Example 6.

### Synthesis of NB-PMMA Macromonomer by ATRP

NB-PMMA macromonomers with different degree of polymerization (DP) were prepared by ATRP. A 25 mL Schlenk tube was charged with norbornenyl-functionalized initiator (53 mg, 0.143 mmol), MMA (1.06 mL, 10.0 mmol), anisole (1.0 mL) and TMEDA (0.011 mL, 0.072 mmol). The solution was degassed by three freeze-pump-thaw cycles. During the final cycle, the Schlenk tube was filled with nitrogen, and CuBr (10.3 mg, 0.072 mmol) was quickly added to the frozen reaction mixture. The Schlenk tube was sealed, evacuated, and backfilled with nitrogen three times. The Schlenk tube was thawed to room temperature and the polymerization was conducted in a 70° C. oil bath for 3 h. The mixture was filtered through neutral alumina, precipitated into MeOH and filtered. The white powder was washed with MeOH followed by drying in vacuum oven overnight. ¹H NMR (500 MHz, CDCl₃): δ 6.30 (s, 2H), 4.17 (m, 2H), 3.76 (m), 3.65-3.59 (m, PMMA), 3.28 (s, 2H), 2.72 (s, 2H), 2.00-1.69 (m, PMMA), 1.07-0.75 (m, PMMA). GPC analysis (THF): Mₙ=5,158, PDI=1.13.

### Typical Procedure for ROMP of NB-PMMA Macromonomer and NBPEG₃₄₀₀(GPHyp)₃ Macromonomer as Representative Prep for PMMA-Peptide Type Copolymers for PEG of MW 1,000-6,000

NBPEG₃₄₀₀(GPHyp)₃ macromonomer (0.1 eq.) was weighed into a 4 ml scintillation vial followed by addition of NB-PMMA (0.05 g). THF (0.02 M wrt. NB-PMMA) was added and the mixture stirred at 45° C. till a clear solution was obtained. A solution of catalyst 2 in THF (1.25 mol %) was added to the solution and the reaction was stirred at r.t. for 2 h. Ethyl vinyl ether was added to the reaction mixture followed by MeOH (3 ml) and the mixture placed in the freezer for 1 h to give a white precipitate. The mother liquor was decanted and the residue washed repeatedly with MeOH followed by drying in vacuum oven.

¹H NMR (500 MHz, CDCl₃): 3.65-3.59 (m, PMMA and PEG), 2.00-1.69 (m, PMMA), 1.07-0.75 (m, PMMA). GPC analysis (THF): Mₙ=65,600, PDI=1.37.

### Applications

The present disclosure provides a new modular synthesis method to create bioactive macromonomers rapidly for construction of bioactive copolymers with synthetic polymer of choice. Bioactive macromonomers may be easily copolymerized with another synthetic copolymer to form bioactive polymers with desired physical and mechanical properties. Advantageously, there is increased stability of bioactive molecule upon connection to polymer linker. Embodiments of the strategy disclosed herein allow for any peptide, carbohydrate or drug molecule to be used in polymer synthesis without loss of bioactivity. Embodiments of the strategy disclosed herein also allow rapid build up of bioactive macromonomer library. Any bioactive molecule with a carboxylic acid group may be used. In summary, the present disclosure provides a highly versatile strategy for biomedical material customization.

Embodiments of the method disclosed herein allow macromonomers to be paired with synthetic polymer of choice to create bioactive polymer that has both mechanical and physical properties of synthetic polymer and biological activity of bioactive molecule.

Embodiments of the method disclosed herein is an easy strategy to create different types of bioactive polymers that are chemically bonded instead of physical blends of bioactive molecules into synthetic polymers.

Advantageously, non-cell or growth factor-based bioactivity is/are provided on the polymer disclosed herein. Embodiments of the bioactive synthetic polymer disclosed herein possess both bioactivity to enhance therapeutic effects such as tissue regeneration, biofilm eradication etc, and also structural integrity and mechanical strength, like a polymer. Embodiments of the bioactive synthetic polymer disclosed herein allow for biomolecule to be blended into base material of synthetic polymer similar to the synthetic polymer side arms of copolymer, without phase separation. Embodiments of the method disclosed herein allow the synthetic polymer to become biocompatible to human tissues upon modification with biomolecules. Embodiments of the method disclosed herein allow a wide range of biomolecules to be used to achieve any desired therapeutic effect. Embodiments of the method disclosed herein also allow a good range of synthetic polymers to be used to achieve different mechanical, physical properties required in material for targeted biodevice.

Embodiments of the bioactive synthetic polymers disclosed herein may be used as bioadditives for biomedical devices to provide therapeutic effects to device material itself.

Embodiments of the method disclosed herein use non cell- or growth factor-based therapy, which allow for long shelf life of device or materials such as scaffold and prevent unwanted or uncontrolled bioactivity (for e.g., tissue regeneration).

Embodiments of the bioactive synthetic polymers disclosed herein may be used as bioadditives for skin or bone scaffold to create stimulus required for skin or bone tissue regeneration.

Embodiments of the bioactive synthetic polymer disclosed herein may be used in bone scaffolds to make PCL more “bone-like” and more biocompatible as a result. Studies have shown that osteocytes do not bind to PCL and only start binding to PCL after collagen is coated on PCL.

The present disclosure also provides a bioactive polyamide-peptide brush polymer that possesses both bioactivity to enhance biocompatibility and wound healing, together with structural integrity and mechanical strength. Embodiments of the polyamide-peptide brush polymers can be blended into polymers similar to the synthetic side chains as bioadditives, to create materials for use in medical devices such as catheters, plastic surgery implants, prosthetic parts, cartilage joint implants. Advantageously, embodiments of the bioactive polyamide-peptide brush polymer disclosed herein can be sterilized by heat before implantation and is long lived. Embodiments of the bioactive polyamide-peptide brush polymer disclosed herein allow product customization by 3DP as it is thermally stable. Embodiments of the bioactive polyamide-peptide brush polymer disclosed herein improve biocompatibility of polyamide which can trigger inflammatory response in body.

The present disclosure also provides a bioactive polystyrene made to be bactericidal whilst still possessing structural integrity and mechanical strength, like a polymer. In various embodiments, antibiotics are attached on the polymer by covalent bonding, therefore preventing leaching of antibiotics into media which can escape into environment if disposal is improperly managed. In various embodiments, there are active sites on the antibiotic molecule left exposed on polymer chain to allow bacterial cell penetration or bacteria RNA binding. Embodiments of the bioactive synthetic polymer disclosed herein allow for antibiotics to be blended into base material of synthetic polymer similar to the synthetic polymer side arms of copolymer, without phase separation. Embodiments of the antibiotic-polystyrene copolymers may be used as bioadditives for biomedical devices to provide bactericidal effect on device without additional drugs added.

The present disclosure also provides a bioactive poly(lactic-co-glycolic acid) that may be used as bioadditives in cartilage implant material fabrication. For example, the bioactive poly(lactic-co-glycolic acid) may be an acellular biodegradable cartilage scaffold with chondrocyte binding capability for cartilage regeneration. Embodiments of the polymer disclosed herein incorporate acellular implant material, therefore providing a lower regulatory handle and a faster path to market. In various embodiments, bioactivity is localized as biomolecules are covalently bonded to synthetic polymer and cannot leach out. In various embodiments therefore, premature metabolism of sulfated saccharides or unintended BMP binding elsewhere in the body is prevented. In various embodiments, biomolecules bound on polymers are able to bind BMP while staying immobilized on scaffold instead of leaching to other parts of body for undesirable side effects or being metabolized prematurely. Embodiments of the bioactive poly(lactic-co-glycolic acid) may be made to be more like polymer used in base material for device fabrication to allow effective blending of biomolecules into main polymer matrix. In various embodiments, phase separation of the bioactive poly(lactic-co-glycolic acid) is unlikely. Advantageously, in various embodiments, biomolecules show improved thermal stability on binding to polymer, allowing for material processing. For example, the polymer is 3D-printable by fused filament fabrication, fused deposition modelling and/or customized into a scaffold. Embodiments of the bioactive synthetic polymer disclosed herein allow for peptides and oligosaccharides to be blended into base material of synthetic polymer similar to the synthetic polymer side arms of copolymer, without phase separation.

The present disclosure also provides PMMA-peptide brush polymers that may be blended with base polymer PMMA as bioadditives, to create materials for use in medical devices such as orthopedic or cranial implants. Embodiments of the PMMA-peptide brush polymers disclosed herein allow implant customization and pre-operative fabrication by 3D printing, therefore improving “fit” and reducing surgical time. Embodiments of the PMMA-peptide brush polymers disclosed herein also improve biocompatibility of PMMA and reduce inflammatory response in body. In various embodiments, the biomolecules are covalently bonded to synthetic polymer and cannot leach out.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A bioactive synthetic copolymer with a poly(norbornene) backbone comprising one or more repeating units represented by general formula (I) and one or more repeating units represented by general formula (II): 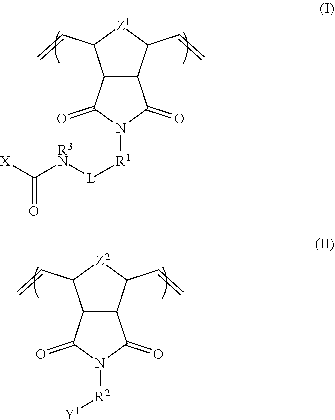
wherein
R¹ is optionally substituted alkyl;
R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R³ is selected from H, optionally substituted alkyl, optionally substituted alkenyl or optionally substituted alkynyl;
L is heteroalkylene;
X comprises a bioactive moiety selected from the group consisting of proteins, peptides, carbohydrates, therapeutic/drug molecules and derivatives thereof;
Y¹ comprises a synthetic polymer or parts thereof; and
Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

2. The copolymer of claim 1, wherein the molecular weight of general formula (I) do not differ from the molecular weight of general formula (II) by more than 30% of the molecular weight of general formula (II).

3. The copolymer of claim 1, wherein L is heteroalkylene having from 20 carbon atoms to 300 carbon atoms.

4. The copolymer of claim 1, wherein L is polyethylene glycol (PEG), optionally wherein L is polyethylene glycol (PEG) having a number average molecular weight of between 500 and 7,000.

5. (canceled)

6. The copolymer of claim 1, wherein R¹ is C₁-C₄ alkyl, and R² is selected from C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl or C₃-C₂₀ alkylcarbonylalkyl, optionally wherein R¹ is straight or branched C₁-C₄ alkyl substituents independently selected from methyl, ethyl, n-propyl, 2-propyl, isopropyl, n-butyl, isobutyl, sec-butyl or t-butyl, and R² is straight or branched C₁-C₂₀ alkyl substituents independently selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl or decyl.

7. (canceled)

8. The copolymer of claim 1, wherein Z¹ and Z² are both CR^{a}R^{b} wherein R^{a} and R^{b} are each independently selected from the group consisting of H, optionally substituted alkyl, optionally substituted alkenyl and optionally substituted alkynyl.

9. The copolymer of claim 1, wherein X comprises protein, peptide or carbohydrate selected from the group consisting of peptide sequence, laminin-derived peptide, integrin binding peptide, cell-penetrating peptide, collagen mimics, collagen fragments, heparin sulfate, glycosaminoglycans (GAGs) and derivatives thereof.

10. The copolymer of claim 1, wherein X is selected from the group consisting of RGD, SRGDS (SEQ ID NO: 1), RGDS (SEQ ID NO: 2), A5G81 (AGQWHRVSVRWGC) (SEQ ID NO: 3), SVVYGLR (SEQ ID NO: 4), (IRIK)₂ (SEQ ID NO: 6), (IKKI)₃ (SEQ ID NO: 7), heparin oligosaccharide DP8, DP10, DP12, DP14, DP16, DGEA (SEQ ID NO: 5), (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence and hyaluronic acid.

11. The copolymer of claim 1, wherein X comprises an antibiotic, antimicrobial, antibacterial moiety, blood thinning agents or anti-inflammatory agents, optionally wherein X is selected from the group consisting of penicillin, amoxicillin, amphotericin, ciprofloxacin (CIF), atorvastatin, aspirin, streptomycin, ribostamycin and gentamycin.

12. (canceled)

13. The copolymer of claim 1, wherein Y¹ is represented by general formula (III): 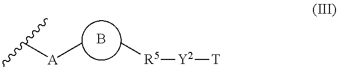
wherein
A is selected from a single bond, oxy, carbonyl, oxycarbonyl, carboxyl, optionally substituted alkoxy, optionally substituted alkoxyalkyl, optionally substituted alkylcarbonyl, optionally substituted alkylcarbonylalkyl, optionally substituted carboxyalkyl, optionally substituted oxycarbonylalkyl, optionally substituted alkylcarboxylalkyl, or optionally substituted alkoxycarbonylalkyl;
B is optionally present as a ring selected from 1,2,3-triazole or succinimide;
R⁵ is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
Y² is selected from the group consisting of polypropylene (PP), polyesters, poly(lactic acid) (PLA), poly(lactic-co-glycolic acid) (PLGA), poly(caprolactone) (PCL), polystyrene (PS), polyacrylates, poly(meth)acrylates, polyamides (PA), and parts thereof, and
T is a terminal group selected from the group consisting of hydrogen, halogen, hydroxyl, amino, acyl, thiol, optionally substituted alkyl, optionally substituted alkenyl, optionally substituted alkynyl, optionally substituted alkoxy, optionally substituted alkoxyalkyl, optionally substituted alkylcarbonyl, optionally substituted alkylcarbonylalkyl, optionally substituted carboxyalkyl, optionally substituted oxycarbonylalkyl, optionally substituted alkylcarboxylalkyl or optionally substituted alkoxycarbonylalkyl.

14. The copolymer of claim 1, wherein Y¹ is selected from the following general formulae (IIIa), (IIIb), (IIIc), (IIId), (IIIe) or (IIIf):

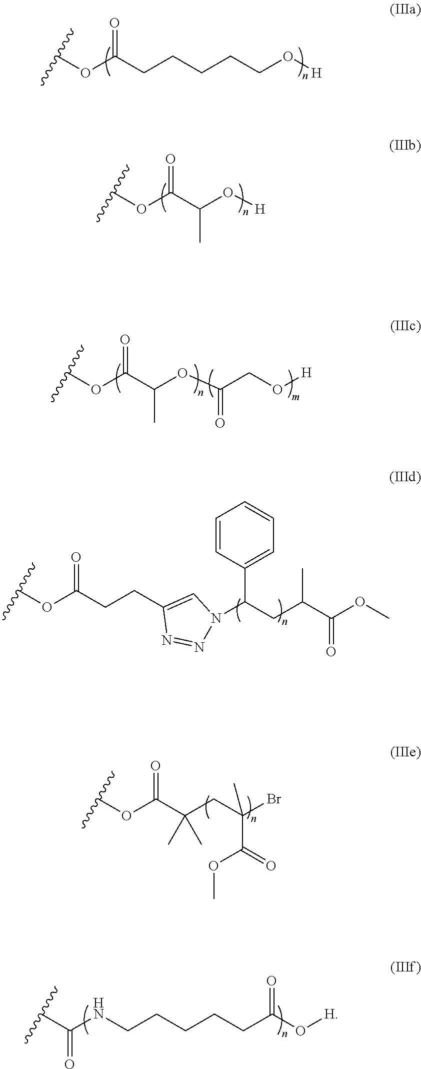

15. A method of preparing a bioactive synthetic copolymer of claim 1, the method comprising:
polymerising one or more bioactive macromolecules represented by general formula (IV) with one or more synthetic macromolecules represented by general formula (V) in the presence of a catalyst to obtain the bioactive synthetic copolymer: 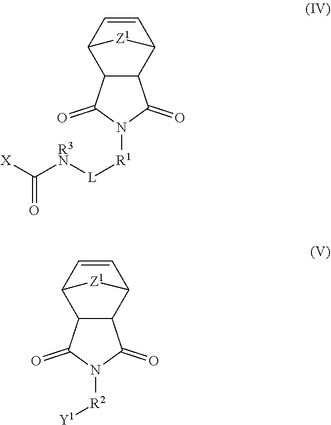
wherein
R¹ is optionally substituted alkyl;
R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R³ is selected from H, optionally substituted alkyl, optionally substituted alkenyl or optionally substituted alkynyl;
L is heteroalkylene;
X comprises a bioactive moiety selected from the group consisting of proteins, peptides, carbohydrates, therapeutic/drug molecules and derivatives thereof;
Y¹ comprises a synthetic polymer or parts thereof; and
Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl.

16. The method according to claim 15, wherein the catalyst comprises a ruthenium complex.

17. The method according to claim 15, wherein the method comprises ring opening metathesis polymerisation (ROMP).

18. (canceled)

19. The method according to claim 15, wherein the method further comprises, prior to polymerizing preparing a bioactive macromolecule by:
(i) providing a dicarboxylic anhydride having general formula (VI): 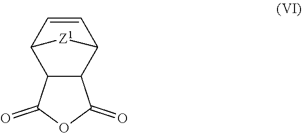
wherein
Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl;
(ii) reacting said dicarboxylic anhydride having general formula (VI) with a diamine R⁴R³N-L-R¹—NH₂ to obtain an amine having general formula (VII): 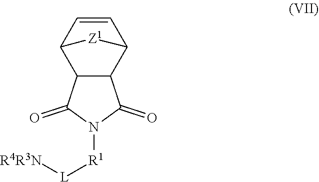
wherein
R¹ is optionally substituted alkyl;
R³ and R⁴ are each independently selected from H, optionally substituted alkyl, optionally substituted alkenyl or optionally substituted alkynyl, wherein at least one of R³ and R⁴ is H;
L is heteroalkylene; and
Z¹ is selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl; and
(iii) reacting said amine having general formula (VII) with an acid-containing bioactive moiety X—C(═O)OH to obtain the bioactive macromolecule, wherein X comprises a bioactive moiety selected from the group consisting of proteins, peptides, carbohydrates, therapeutic/drug molecules and derivatives thereof.

20. The method according to claim 19, wherein the method further comprises, prior to reacting amine having general formula (VII) with X—C(═O)OH, purifying the amine having general formula (VII) to remove impurities.

21. The method according to claim 20, wherein purifying comprises double neutralisation.

22. The method according to claim 21, wherein the double neutralisation comprises a first washing with acid and a second washing with base.

23. A material comprising a copolymer of claim 1 for use in medicine.

24. The material according to claim 23, wherein the material is part of an apparatus selected from the group consisting of wound dressing, skin scaffold, bone scaffold, organoid scaffold, implants, and medical device.
